(19) United States
(12) Patent Application Publication
(10) Pub. No.: US 2025/0302925 A1
(43) Pub. Date: Oct. 2, 2025

(54) CONJUGATED CRISPR-CAS COMPLEXES

(71) Applicant: Synthego Corporation, Redwood City, CA (US)

(72) Inventors: Travis MAURES, La Jolla, CA (US); Jared CARLSON-STEVERMER, Burlingame, CA (US); Sahil JOSHI, Somerville, MA (US); Reed KELSO, San Francisco, CA (US); Anastasia KADINA, Sunnyvale, CA (US); John Andrew WALKER, II, San Leandro, CA (US)

(21) Appl. No.: 18/833,834

(22) PCT Filed: Jan. 27, 2023
(86) PCT No.: PCT/US2023/061463

§ 371 Date: Jul. 26, 2024

Related U.S. Application Data
(60) Provisional application No. 63/303,974, filed on Jan. 27, 2022.
(60) Provisional application No. 63/327,451, filed on Apr. 5, 2022.

(51) Int. Cl. A61K 38/46; A61K 31/7088; C12N 9/22; C12N 15/11

(52) U.S. Cl. CPC A61K 38/465; A61K 31/7088; C12N 9/226; C12N 15/11; C12N 2310/20

(57) ABSTRACT

Provided herein are polynucleotides and CRISPR effector proteins configured to be covalently bound together in a CRISPR complex. The polynucleotides can be further modified to modulate the activity of the CRISPR complex. Modification of the polynucleotide and CRISPR effector protein can be used to improve the efficacy of target binding and/or cleavage.

### CROSS-REFERENCE TO RELATED PATENT APPLICATIONS

The present application is a national stage filing under 35 U.S.C. 371 of International Application No. PCT/US2023/061463, filed Jan. 27, 2023, which claims the benefit of priority under 35 U.S.C. § 119(e) to U.S. Provisional Application No. 63/303,974, filed on Jan. 27, 2022, and U.S. Provisional Application No. 63/327,451 filed on Apr. 5, 2022, the contents of which are incorporated herein by reference in their entirety.

### REFERENCE TO AN ELECTRONIC SEQUENCE LISTING

The contents of the electronic sequence listing (17477400137.xml; Size: 269,718 bytes; and Date of Creation: Jan. 17, 2025) is herein incorporated by reference in its entirety

### BACKGROUND

CRISPR/Cas can be used in various medical, laboratory and other exploratory settings. The CRISPR/Cas system can be used as a gene editing tool in a plethora of different organisms to generate breaks at a target site and subsequently introduce mutations at the locus. Two main components can be needed for the gene editing process: an endonuclease-like Cas enzyme and a short RNA molecule to recognize a specific DNA target nucleic acid sequence. Instead of engineering a nuclease enzyme for every DNA target, the CRISPR/Cas system can rely on customized short RNA molecules to recruit the Cas enzyme to a different nucleic acid, e.g., DNA, target site. Examples of Cas enzymes include Cas9 and Cpf1. Synthetic guide RNAs, e.g., single guide RNAs (sgRNAs), used to form CRISPR complexes can be subject to degradation when not in complex with a Cas enzyme. Synthetic guide RNAs, e.g., single guide RNAs (sgRNAs), used to form CRISPR complexes can induce an immune response which can limit the application of currently available sgRNA/Cas nuclease complexes. CRISPR complexes can dissociate in vivo either partially or fully which can reduce efficiency and possibly cause off target cleavage events. Due to the instability of CRISPR complexes, they are often delivered encoded in a plasmid which relies on the transcription of the target cell to produce the encoded protein and guide sequence. There is a need for delivery of precise ratios of CRISPR Cas enzyme and guide RNA molecules that are consistent in any research context, such as the delivery of a pure reagent in a controlled dosing regimen. Additionally, there is a need for CRISPR complexes with enhanced stability for use in various settings requiring, for example, precise dosing of one or more exogenous CRISPR complexes with tunable activity.

### SUMMARY

Disclosed herein is a CRISPR complex comprising a single guide RNA (sgRNA) conjugated to a CRISPR effector protein at an unnatural nucleotide within the sgRNA, wherein the sgRNA comprises a crRNA region and a tracrRNA region, and wherein the unnatural nucleotide is outside a target binding region of the crRNA region. The unnatural nucleotide can comprise a uracil or thymidine having a modification through which the uracil or thymidine is conjugated to the CRISPR effector protein. The unnatural nucleotide can be at nucleotide position 49 of the sgRNA, wherein nucleotide position 1 is at a 5′ end of the target binding region of the crRNA and nucleotide positions of the sgRNA are numbered consecutively from 5′ to 3′ from nucleotide position 1. The unnatural nucleotide can comprise a modified sugar. The unnatural nucleotide can comprise a modified base. The unnatural nucleotide can comprise a maleimide. The maleimide can covalently link to a cysteine on the CRISPR effector protein thereby conjugating the unnatural nucleotide to the CRISPR effector protein. The unnatural nucleotide can comprise pyridyl disulfide, alkoxyamine, NHS ester, diazarine, imidoester, haloacetyl group, hydrazide, aryl azide, isocyanate, dithiol phosphoramidite DTPA, 4-thio-UTP, 5-azido-UTP, 5-bromo-UTP, 8-azido-ATP, 5-APAS-UTP, or 8-N(3)AMP.

In some embodiments, the unnatural nucleotide can be in a stem loop of the tracrRNA region of the sgRNA. A structure of the stem loop can be maintained relative to a structure of a stem loop of a sgRNA lacking the unnatural nucleotide. The unnatural nucleotide can be in a bulge of the tracrRNA region. A structure of the bulge can be maintained relative to a structure of a bulge of a sgRNA lacking the unnatural nucleotide. The unnatural nucleotide can be between stem loops of the tracrRNA region. The CRISPR complex can comprise nuclease activity. In some embodiments, an off-target nuclease activity of the CRISPR complex is equal to or less than an off-target nuclease activity of a CRISPR complex comprising the CRISPR effector protein and the sgRNA that are not conjugated. The unnatural nucleotide can be within 20 angstroms of a cysteine of the CRISPR effector protein. In some embodiments, the unnatural nucleotide can not be 4-thiouridine or a modified adenosine.

Further disclosed herein is a CRISPR complex comprising a single guide RNA (sgRNA) conjugated to a CRISPR effector protein at nucleotide position 49 of the sgRNA, wherein nucleotide position 1 is at a 5′ end of the target binding region of the crRNA and nucleotide positions of the sgRNA are numbered consecutively from 5′ to 3′ from nucleotide position 1. The nucleotide at nucleotide position 49 can comprise a uracil or thymidine having a modification through which the uracil or thymidine is conjugated to the CRISPR effector protein. The CRISPR complex can comprise nuclease activity. In some embodiments, the CRISPR complex can comprise a single guide RNA (sgRNA) conjugated to a CRISPR effector protein at an unnatural nucleotide within the sgRNA, wherein the CRISPR complex comprises nuclease activity.

Disclosed herein is a pharmaceutical formulation comprising the CRISPR complex and a pharmaceutically acceptable excipient. Further disclosed is a method comprising administering the pharmaceutical formulation to a subject.

Disclosed herein is a method comprising introducing the CRISPR complex into a cell. Also disclosed is a kit comprising the CRISPR complex and instructions.

Disclosed herein is a method of editing a nucleic acid molecule comprising contacting the CRISPR complex to a nucleic acid molecule. The CRISPR complex can comprise an off-target cleavage activity of less than 2% of cleavage events.

Disclosed herein is a method of editing a target gene in a plurality of cells comprising administering the CRISPR complex to a plurality of cells comprising a target gene, thereby generating cells comprising edited target genes, wherein 99% of the cells comprising edited target genes remain viable after administration of the CRISPR complex. Cell viability can be measured by resazurin assay.

Disclosed herein is a method of producing a CRISPR complex comprising conjugating a sgRNA comprising a crRNA region and a tracrRNA region to a CRISPR effector protein, wherein the conjugating occurs at an unnatural nucleotide outside the crRNA region of the sgRNA, wherein nuclease activity of the CRISPR effector protein is maintained after the conjugating. The unnatural nucleotide can comprise a uracil or thymidine having a modification through which the uracil or thymidine is conjugated to the CRISPR effector protein. The unnatural nucleotide can comprise a maleimide. The conjugating can be between the uracil or thymidine and a cysteine on the CRISPR effector protein via a linking moiety. The uracil can comprise a 4-thio uridine. The thymidine can comprise 4-thio thymidine. The conjugating can be between the uracil or thymidine and an amine group on the CRISPR effector protein via a linking moiety. The uracil can comprise a 5-bromo uridine. The conjugating can occur in solution, and a ratio of the sgRNA to the CRISPR effector protein in the solution can be at least 9:1. The crosslinking can comprise exposing the solution to UV light. The crosslinking can occur upon mixing of the sgRNA with the CRISPR effector protein.

Disclosed herein is a method comprising conjugating a single guide RNA (sgRNA) comprising an unnatural nucleotide to a CRISPR effector protein using a conjugating agent, wherein the conjugating occurs at the unnatural nucleotide outside a target binding region of the sgRNA, thereby generating a cross-linked complex, wherein the cross-linked complex comprises nuclease activity.

Disclosed herein is a single guide RNA (sgRNA) comprising a crRNA region and a tracrRNA region and an unnatural nucleotide at nucleotide position 49, wherein nucleotide position 1 is at a 5′ end of a target binding region of the crRNA region and nucleotide positions of the sgRNA are numbered consecutively from 5′ to 3′ from nucleotide position 1.

Disclosed herein is a single guide RNA (sgRNA) comprising a crRNA region and a tracrRNA region and a uracil or thymidine at nucleotide position 49, wherein nucleotide position 1 is at a 5′ end of a target binding region of the crRNA region and nucleotide positions of the sgRNA are numbered consecutively from 5′ to 3′ from nucleotide position 1. The uracil or thymidine may comprise a modification through which the uracil or thymidine may be conjugated to a CRISPR effector protein.

Disclosed herein is a CRISPR complex comprising a single guide RNA (sgRNA) conjugated to a CRISPR effector protein, wherein the sgRNA comprises a crRNA region, a tracrRNA region, and a sequence configured to modulate activity of the CRISPR complex. The sgRNA can be a CRISPR ON polynucleotide, CRISPR OFF polynucleotide, CRISPR ON/OFF polynucleotide, or CRISPR polynucleotide modified to decrease off-target editing. The sgRNA can comprise an unnatural nucleotide within the sgRNA, and sgRNA may be conjugated to the CRISPR effector protein at the unnatural nucleotide. The unnatural nucleotide can be outside a target binding region of the crRNA region. The unnatural nucleotide can be at nucleotide position 49 of the sgRNA, wherein nucleotide position 1 is at a 5′ end of the target binding region of the crRNA and nucleotide positions of the sgRNA are numbered consecutively from 5′ to 3′ from nucleotide position 1. The unnatural nucleotide can comprise a modified sugar. The unnatural nucleotide can comprise a modified base. The unnatural nucleotide can comprise a maleimide. The maleimide can covalently link to a cysteine on the CRISPR effector protein thereby conjugating the unnatural nucleotide to the CRISPR effector protein. The unnatural nucleotide can comprise pyridyl disulfide, alkoxyamine, NHS ester, diazarine, imidoester, haloacetyl group, hydrazide, aryl azide, isocyanate, dithiol phosphoramidite DTPA, 4-thio-UTP, 5-azido-UTP, 5-bromo-UTP, 8-azido-ATP, 5-APAS-UTP, or 8-N(3)AMP.

The unnatural nucleotide of the sgRNA can be in a stem loop of the tracrRNA region of the sgRNA. A structure of the stem loop can be maintained relative to a structure of a stem loop of a sgRNA lacking the unnatural nucleotide. The unnatural nucleotide can be in a bulge of the tracrRNA region. A structure of the bulge can be maintained relative to a structure of a bulge of a sgRNA lacking the unnatural nucleotide. The unnatural nucleotide can be between stem loops of the tracrRNA region. The CRISPR complex can comprise nuclease activity. An off-target nuclease activity of the CRISPR complex can be equal to or less than an off-target nuclease activity of a CRISPR complex comprising the CRISPR effector protein and the sgRNA that are not conjugated. The unnatural nucleotide can be within 20 angstroms of a cysteine of the CRISPR effector protein. In some embodiments, the unnatural nucleotide may not be 4-thiouridine or a modified adenosine.

Disclosed herein is a polynucleotide comprising a modification, wherein the polynucleotide comprises: (i) a guide sequence configured to anneal to a target sequence in a target nucleic acid molecule (ii) a sequence configured to bind to a CRISPR effector protein and comprising the modification, and (iii) an unnatural nucleotide configured to cross-link to a CRISPR effector protein; wherein when the polynucleotide is complexed with a CRISPR effector protein, a first CRISPR complex is formed having a lower editing activity of an off-target nucleic acid molecule than a second CRISPR complex comprising the polynucleotide without the modification complexed with the CRISPR effector protein. The unnatural nucleotide can be at position 49. The modification can comprise a linker not comprising a canonical nucleotide base. The modification can comprise at least two linkers not comprising a canonical nucleotide base. The sequence of ii) can form, from 5′ to 3′, a tetraloop, a first stem loop, a second stem loop, and a third stem loop. In some instances, the polynucleotide does not comprise a fourth stem loop. In some instances, the polynucleotide does not comprise a stem loop at a 5′ end of the polynucleotide. The linker can comprise a cleavable linker. The linker can comprise 3-(4,4′-Dimethoxytrityl)-1-(2-nitrophenyl)-propan-1-yl-[(2-cyanoethyl)-(N,N-diisopropyl)]-phosphoramidite. The linker can comprise a photolabile linker. The photolabile linker can be cleavable by ultraviolet radiation. The photolabile linker can be cleavable by visible light. The cleavable linker can comprise 3-(4,4′-Dimethoxytrityl)-1-(2-nitrophenyl)-propan-1-yl-[(2-cyanoethyl)-(N,N-diisopropyl)]phosphoramidite. The cleavable linker can comprise 1-(7-(diethylamino)-2-oxo-2H-chromen-4-yl)propyl. The cleavable linker can comprise

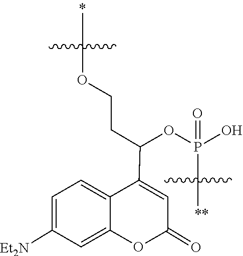

wherein * indicates a point of attachment to H, or a first nucleotide and ** indicates a point of attachment to OH, or a second nucleotide. The photolabile linker can comprise phosphoramidite. The photolabile linker can comprise coumarin. The modification can be at position 57 or position 74 of the polynucleotide, wherein position 1 is at a 5′ end of the polynucleotide, and positions are counted from 5′ to 3′. The modification can be at position 57 and position 74 of the polynucleotide. The modification can be in a loop. The modification can be in the first stem loop or the second stem loop. The modification can be in a loop of the first stem loop or a loop of the second stem loop. The modification can be at one or both of positions 57 and 74, wherein position 1 is at a 5′ end of the polynucleotide, and positions are counted from 5′ to 3′. The modification can comprise a photo cleavable bond. In some instances, the modification is not in a stem loop. The polynucleotide can comprise 2′-O-methyl analogs and 3′phosphorothioate inter nucleotide linkages at a first three 5′ and 3′ terminal RNA nucleotides. Editing activity can be measured as a percentage of off-target nucleic acid molecules that are edited. The editing activity of the off-target nucleic acid molecules by the first CRISPR complex can be lower that an editing activity of the second CRISPR complex with a p-value≤0.0001. The editing activity of the first CRISPR complex of the target nucleic acid molecule and an editing activity of the second CRISPR complex of the target nucleic acid molecule can be within 5%. The editing activity of the first CRISPR complex of the target nucleic acid molecule and the editing activity of the second CRISPR complex of the target nucleic acid molecule can be measured as a percentage of target nucleic acid molecules that are edited. Disclosed herein is a CRISPR complex comprising any of the aforementioned polynucleotides and a CRISPR enzyme. The CRISPR complex can comprise nuclease activity.

In another aspect, described herein, is a nucleotide or oligonucleotide comprising a linker of Formula (I):

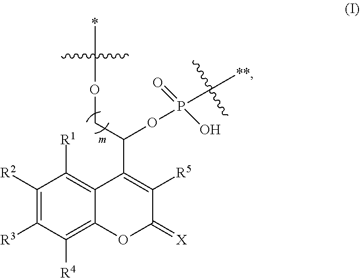

wherein: R¹, R², R³, R⁴, and R⁵ are each independently selected from H, alkyl, substituted alkyl, alkoxy, alkenyl, alkynyl, haloalkyl, haloalkoxy, alkoxyalkyl, amino, aminoalkyl, halo, cyano, hydroxy, hydroxyalkyl, heteroalkyl, C-carboxy, O-carboxy, C-amido, N-amido, nitro, sulfonyl, sulfo, sulfino, sulfonate, S-sulfonamido, N-sulfonamido, optionally substituted carbocyclyl, optionally substituted aryl, optionally substituted heteroaryl and optionally substituted heterocyclyl; alternatively, two or more of R¹, R², R³, and R⁴, together with the atoms to which they are attached form a ring or ring system selected from optionally substituted 5- to 10-membered heteroaryl, optionally substituted 5- to 10-membered heterocyclyl, and optionally substituted C₅₋₁₀ carbocycle; m can be an integer selected from 1 to 10; X can be selected from O, S, ═C(CN)2; * can indicate a point of attachment to H, or a pentose moiety; and ** can indicate a point of attachment to OH, or a phosphate group of a nucleotide. The linker of Formula (I) can be represented by Formula (I′):

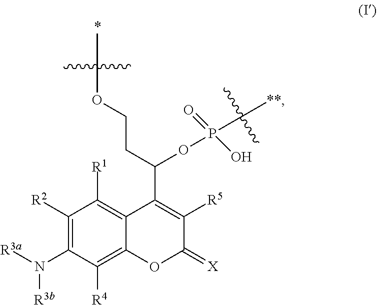

wherein: R¹, R², R^{3a}, R^{3b}, R⁴, and R⁵ are each independently selected from the group consisting of H, alkyl, substituted alkyl, alkoxy, alkenyl, alkynyl, haloalkyl, haloalkoxy, alkoxyalkyl, amino, aminoalkyl, halo, cyano, hydroxy, hydroxyalkyl, heteroalkyl, C-carboxy, O-carboxy, C-amido, N-amido, nitro, sulfonyl, sulfo, sulfino, sulfonate, S-sulfonamido, N-sulfonamido, optionally substituted carbocyclyl, optionally substituted aryl, optionally substituted heteroaryl and optionally substituted heterocyclyl; alternatively, two or more of R², R^{2a}, R^{3a}, and R⁴, together with the atoms to which they are attached form a ring or ring system selected from optionally substituted 5- to 10-membered heteroaryl, optionally substituted 5- to 10-membered heterocyclyl, and optionally substituted C₅₋₁₀ carbocycle; X can be oxygen, S, or ═C(CN)2. R¹, R², R⁴, and R⁵ can each independently be H or C₁₋₆ alkyl; and R^{3a}, and R^{3b} can be C₁₋₆ alkyl. R¹, R², R⁴, and R⁵ can each be H; and R^{3a}, and R^{3b} can each be ethyl.

In another aspect, provided herein is a compound comprising

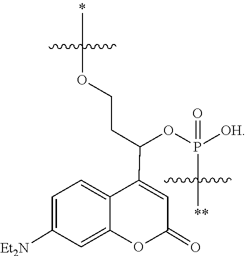

Disclosed herein is a polynucleotide comprising the aforementioned compound. The polynucleotide can further comprise a sequence configured to bind a CRISPR enzyme. The polynucleotide can further comprise a guide sequence configured to anneal to a target sequence in a target nucleic acid molecule. Disclosed herein is a CRISPR complex comprising a CRISPR enzyme and an aforementioned polynucleotide.

In another aspect, described herein, is a compound comprising Formula (I):

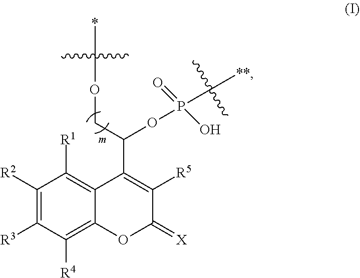

wherein: R¹, R², R³, R⁴, and R⁵ are each independently selected from H, alkyl, substituted alkyl, alkoxy, alkenyl, alkynyl, haloalkyl, haloalkoxy, alkoxyalkyl, amino, aminoalkyl, halo, cyano, hydroxy, hydroxyalkyl, heteroalkyl, C-carboxy, O-carboxy, C-amido, N-amido, nitro, sulfonyl, sulfo, sulfino, sulfonate, S-sulfonamido, N-sulfonamido, optionally substituted carbocyclyl, optionally substituted aryl, optionally substituted heteroaryl and optionally substituted heterocyclyl; alternatively, two or more of R¹, R², R³, and R⁴, together with the atoms to which they are attached form a ring or ring system selected from optionally substituted 5- to 10-membered heteroaryl, optionally substituted 5- to 10-membered heterocyclyl, and optionally substituted C₅₋₁₀ carbocycle; m can be an integer selected from 1 to 10; X can be selected from O, S, ═C(CN)2; * can indicate a point of attachment to H, or a pentose moiety; and ** can indicate a point of attachment to OH, or a phosphate group of a nucleotide. The compound of Formula (I) can be represented by Formula (I′):

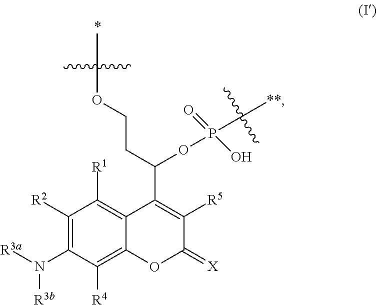

wherein: R¹, R², R^{3a}, R^{3b}, R⁴, and R⁵ are each independently selected from the group consisting of H, alkyl, substituted alkyl, alkoxy, alkenyl, alkynyl, haloalkyl, haloalkoxy, alkoxyalkyl, amino, aminoalkyl, halo, cyano, hydroxy, hydroxyalkyl, heteroalkyl, C-carboxy, O-carboxy, C-amido, N-amido, nitro, sulfonyl, sulfo, sulfino, sulfonate, S-sulfonamido, N-sulfonamido, optionally substituted carbocyclyl, optionally substituted aryl, optionally substituted heteroaryl and optionally substituted heterocyclyl; alternatively, two or more of R², R^{2a}, R^{3a}, and R⁴, together with the atoms to which they are attached form a ring or ring system selected from optionally substituted 5- to 10-membered heteroaryl, optionally substituted 5- to 10-membered heterocyclyl, and optionally substituted C₅₋₁₀ carbocycle; X can be oxygen, S, or ═C(CN)2. R¹, R², R⁴, and R⁵ can each independently be H or C₁₋₆ alkyl; and R^{3a}, and R^{3b} can be C₁₋₆ alkyl. R¹, R², R⁴, and R⁵ can each be H; and R^{3a}, and R^{3b} can each be ethyl.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a simplified diagram of a CRISPR complex wherein the polynucleotide is a single guide RNA (sgRNA). The star at position 49 indicates an exemplary position of an unnatural nucleotide for conjugating the polynucleotide to the Cas nuclease. The bar indicates the target binding region of the polynucleotide.

FIG. 2 shows a 3-D model of a sgRNA bound to a target sequence; the tetraloop and stem loops are highlighted to relate the diagram to FIGS. 3-5 (images modified from Nishimasu, H., Ishitani, R., & Nureki, O. (2014). Crystal structure of Streptococcus pyogenes Cas9 in complex with guide RNA and target DNA. Cell. doi:10.2210/pdb4oo8/pdb).

FIG. 3 shows a diagram of the interaction of bound sgRNA nucleotides within the nexus and adjacent amino acids of a Cas9 nuclease.

FIG. 4 shows a diagram of the interaction of bound sgRNA nucleotides within stem loop 1 and adjacent amino acids of a Cas9 nuclease.

FIG. 5 shows a diagram of the interaction of bound sgRNA nucleotides within stem loop 2 and adjacent amino acids of a Cas9 nuclease.

FIG. 6 shows a crystal structure showing exemplary RNA nucleotides to be modified and sites of crosslinking on the protein.

FIG. 7 illustrates the interaction between SpCas9 and sgRNA including the interaction between SpCas9 Cys80 and sgRNA A49 and their relative distance of 12.489 angstroms.

FIG. 8 illustrates a general reaction scheme for modifying a thymidine or uracil of sgRNA to comprise a maleimide for conjugation to a thiol group of a cysteine residue of Cas to prepare a locked sgRNA/Cas complex.

FIG. 9 illustrates a standard sgRNA 100mer sequence (SEQ ID NO: 188) and a modified sequence (SEQ ID NO: 189) in which the U22-A49 pair has been swapped for an A22-Y49 pair. Y is 5′-Dimethoxytrityl-5-[N-(trifluoroacetylaminohexyl)-3-acrylimido]-2′-deoxyUridine, 3′-[(2-cyanoethyl)-(N,N-diisopropyl)]-phosphoramidite.

FIG. 10 provides an illustration of a standard sgRNA sequence and a modified sequence in which the U22-A49 pair has been swapped for an A22-dT49 pair.

FIG. 11 provides a reaction scheme using a heterobifunctional crosslinker to install maleimide moiety in a nucleotide of sgRNA.

FIG. 12 provides a reaction scheme between a maleimide-modified nucleotide of a sgRNA and a cysteine redicue of Cas9.

FIG. 13 illustrates the structure of a portion of the conjugation product after the maleimide moiety of the sgRNA reacts with a thiol of a cysteine residue of Cas9. Shown here is a fragment of the thiol of C80 of Cas9 (N77-R78-I79-(C80)-81Y-82L-83Q) conjugated with a maleimide on a modified nucleotide of sgRNA.

FIG. 14 is a photograph of an agarose gel showing in vitro cleavage of FANCF amplicon by regular RNP and Locked RNP. Lane M: TriDye™ 100 bp DNA Ladder (NEB, N3271S); Lane 1: FANCF amplicon. Lane 2: Digestion of FANCF amplicon by RNP pre-formed with FANCF sgRNA 30 nM (“regular RNP”); Lane 3: Digestion of FANCF amplicon by RNP pre-formed with maleimide-derivatized FANCF sgRNA 30 nM (“Locked RNP”); Lane 4: Digestion of FANCF amplicon by RNP pre-formed with FANCF sgRNA 30 nM (“Regular RNP”) in Lonza buffer; Lane 5: Digestion of FANCF amplicon by RNP pre-formed with maleimide-derivatized FANCF sgRNA 30 nM (“Locked RNP”) in Lonza buffer; Lane 6: Digestion of FANCF amplicon by RNP pre-formed with maleimide-derivatized FANCF sgRNA (“Locked RNP”) at 30 nM in MES buffer; Lanes 7-10: TE Buffer.

FIG. 15 shows in vitro cleavage of RelA amplicon by regular RNP and Locked RNP. Lane M: TriDye™ 100 bp DNA Ladder (NEB N3271S); Lane 1: RelA amplicon; Lane 2: Digestion of RelA amplicon by RNP pre-formed with RelA sgRNA (“Regular RNP”) at 30 nM in water; Lane 3: Digestion of RelA amplicon by RNP pre-formed with maleimide-derivatized RelA sgRNA (“Locked RNP”) at 1.5 nM in water. Lane 4: Digestion of RelA amplicon by RNP pre-formed with maleimide-derivatized RelA sgRNA (“Locked RNP”) at 30 nM in water; Lane 5: Digestion of RelA amplicon by RNP pre-formed with maleimide-derivatized RelA sgRNA (“Locked RNP”) at 600 pM in water; Lane 6: Digestion of RelA amplicon by RNP pre-formed with maleimide-derivatized RelA sgRNA (“Locked RNP”) at 60 pM in water. At 30 nM RelA Locked RNP (lane 4) demonstrates in vitro cleavage of the RelA amplicon comparable to that of the standard RelA RNP (lane 2).

FIG. 16 shows pre-formed RNP complexes analyzed by SDS-PAGE, SYBR Gold stain. Lane 1: Ladder (Precision Plus Protein Dual Color Standards, BioRad 1610374); Lane 2: RelA sgRNA; Lane 3: SpCas9; Lane 4: Maleimide-derivatized RelA sgRNA 1:1 SpCas9 (“Locked RNP”); Lane 5: Maleimide-derivatized RelA sgRNA 2:1 SpCas9 (“Locked RNP”); Lane 6: RelA sgRNA 2:1 SpCas9 (“Regular RNP”). When analyzed with SDS-PAGE, regular RNP complex dissociates, producing a band at ˜150 kDa (lane 6), consistent with the mobility of free SpCas9 protein (lane 3), as well as an sgRNA band (˜30 kDa). Conversely, Locked RNP, featuring a covalent bond between Cas9 and sgRNA, does not dissociate in these conditions, resulting in a visible band shift, corresponding to ˜180 kDa (lane 5).

FIG. 17 and FIG. 18 show profiles of gel lanes 2, 3, 5 and 6 from FIG. 16. Lane 2: RelA sgRNA; Lane 3: SpCas9; Lane 5: Maleimide-derivatized RelA sgRNA 2:1 SpCas9 (“Locked RNP”); Lane 6: RelA sgRNA 2:1 spCas9 (“Regular RNP”). Profiles of the gel lanes (FIG. 16) for additional visualization are shown below each profile. Regular RNP (lane 6) denatures into its components—free Cas9 protein and sgRNA. Locked RNP (lane 5) features an additional band, with mobility lower than that of free Cas9, consistent with the covalently linked Locked RNP species.

FIG. 19 shows pre-formed RNP complexes analyzed by Native PAGE, SYBR Gold stain. Lane 1: Ladder (Low Range ssRNA Ladder NEB N0364S); Lane 2: RelA sgRNA; Lane 3: SpCas9 (not visible); Lane 4: Maleimide-derivatized RelA sgRNA 1:1 SpCas9 (“Locked RNP”); Lane 5: Maleimide-derivatized RelA sgRNA 2:1 SpCas9 (“Locked RNP”); Lane 6: RelA sgRNA 2:1 spCas9 (“Regular RNP”). When analyzed by Native PAGE, Locked and regular RNP migrate through the gel with a similar mobility (lanes 5 and 6, respectively), consistent with the hypothesis that both complexes move intact on the native gel. Free sgRNA can be observed in both lanes.

FIG. 20 and FIG. 21 show profiles of the same gel lanes 2, 3, 5 and 6 from FIG. 19 Lane 2: RelA sgRNA; Lane 3: SpCas9 (not visible); Lane 5: Maleimide-derivatized RelA sgRNA 2:1 SpCas9 (“Locked RNP”); Lane 6: RelA sgRNA 2:1 SpCas9 (“Regular RNP”). Profiles of the gel lanes (FIG. 19) for additional visualization are shown below each profile. The Locked and Regular RNP move intact through the native gel to form bands with the identical shift (lanes 5 and 6). Free SpCas9 (lane 2) is not visible unless complexed with sgRNA.

FIG. 22 is a graph showing in vivo genome editing. Left to right: editing of FANCF and RelA loci in HEK293T cells with regular RNP (lanes 1 and 3) and Locked RNP (lanes 2 and 4) as analyzed by Inference of CRISPR Edits (ICE). In these experiments, the editing efficiency at the FANCF locus is comparable between the regular RNP and Locked RNP.

### DETAILED DESCRIPTION

### I. Overview

Disclosed herein is a polynucleotide (CRISPR polynucleotide) comprising a sequence designed to anneal to a target nucleic acid sequence and a sequence designed to bind a CRISPR effector protein, wherein the CRISPR polynucleotide comprises a moiety for conjugating or crosslinking the CRISPR polynucleotide to a protein. The moiety for conjugating or crosslinking the CRISPR polynucleotide to a protein can be in a hairpin region of the polynucleotide. In another aspect, provided herein is a CRISPR complex comprising the CRISPR polynucleotide and a CRISPR effector protein. The CRISPR polynucleotide can be designed to bind to the CRISPR effector protein, e.g., a Cas enzyme, to form the CRISPR complex. The Cas enzyme can be Cas9, Cas12a, Cas12b, etc. Also provided herein are methods for conjugating the CRISPR polynucleotide to the CRISPR effector protein to form a conjugated CRISPR complex. For example, the CRISPR polynucleotide can be covalently bonded to the Cas enzyme, e.g., through activation of a cross-linking reaction by exposure to a particular wavelength of light in the ultraviolet range or by the positioning of an unnatural nucleotide within the sgRNA which will form a covalent bond upon close proximity to a target amino acid side chain.

In another aspect, provided herein is a CRISPR complex comprising: a) a CRISPR polynucleotide comprising a sequence designed to anneal to a target nucleic acid sequence, a sequence designed to bind a CRISPR effector protein, with or without one or more elements that can be modulated to affect activity; and b) a CRISPR effector protein, wherein an equilibrium dissociation constant (K_{d}) for the CRISPR polynucleotide binding to the CRISPR effector protein is less than 8 pM.

In another aspect, the CRISPR polynucleotides can comprise (i) a sequence configured to covalently bind to a CRISPR effector protein, (ii) optionally, a guide sequence configured to anneal to a target sequence in a target molecule, and (iii) one or more elements that can be modulated to affect the activity of a CRISPR effector protein complexed with the CRISPR polynucleotide. A CRISPR effector protein complexed with the CRISPR polynucleotide can be considered to be “tunable.” In some cases, the one or more elements can be modulated to increase the activity of a CRISPR effector protein complexed with the CRISPR polynucleotide (e.g., CRISPR “ON” complexes). In some cases, the one or more elements can be modulated to decrease the activity of a CRISPR effector protein complexed with the CRISPR polynucleotide (e.g., CRISPR “OFF” complexes). In some cases, a first element in the CRISPR polynucleotide can be modulated to increase the activity of a CRISPR effector protein complexed with the CRISPR polynucleotide and second element can be modulated to decrease the activity of a CRISPR effector protein complexed with the CRISPR polynucleotide (e.g., CRISPR “ON/OFF” complexes).

Also provided herein are complexes comprising a CRISPR effector protein crosslinked to a CRISPR polynucleotide (e.g., CRISPR ON complexes; CRISPR OFF complexes; or CRISPR ON/OFF complexes). In some cases, the cross-link can be at an unnatural nucleotide in the CRISPR polynucleotide. Methods of modulating the CRISPR polynucleotides are provided herein. Kits comprising the polynucleotides and, e.g., instructions, and optionally CRISPR effector protein, are provided. Furthermore, pharmaceutical formulations comprising the CRISPR polynucleotides and a pharmaceutically acceptable excipient are provided, as well as methods of administering the pharmaceutical formulations. Methods of introducing the CRISPR polynucleotides into a cell are also provided herein.

Methods and kits making use of the CRISPR polynucleotides and CRISPR complexes are provided herein. For example, provided herein are methods comprising contacting a target nucleic acid sequence with the CRISPR complex. In addition, provided herein is a pharmaceutical formulation comprising the CRISPR polynucleotide and/or the CRISPR complex and a pharmaceutically acceptable excipient. In another aspect, a method is provided comprising administering the pharmaceutical formulation to a subject. Moreover, provided herein is a method comprising introducing the CRISPR complex into a cell.

Kits comprising the CRISPR polynucleotide and/or CRISPR complex are also provided herein.

### II. CRISPR Overview

Provided herein are CRISPR/Cas complexes with enhanced stability. Provided herein are CRISPR/Cas complexes with enhanced stability and tunable activity. CRISPR (clustered regularly interspaced short palindromic repeats) can be a family of DNA sequences found within the genomes of prokaryotes derived from DNA fragments from viruses previously encountered by the prokaryote. A CRISPR effector protein (e.g., a Cas nuclease) can bind to a CRISPR polynucleotide (e.g., RNA) derived from the DNA sequence, and also a target region: a (viral) DNA sequence complementary to the CRISPR polynucleotide sequence. Upon binding, the Cas nuclease can make a double strand cut in the target region of the target (viral) DNA in order to inactivate it. The target region can comprise a “protospacer” and a “protospacer adjacent motif” (PAM), and both domains can be needed for a Cas enzyme mediated activity (e.g., cleavage). The target site can be adjacent to a PAM site for a nuclease, e.g., Cas9, C2c1, C2c3, or Cpf1. The Cas nuclease can be Cas9. The PAM site can be a short sequence recognized by the CRISPR effector protein and, in some cases, required for the Cas enzyme activity, e.g., the PAM site can be NGG. The sequence and number of nucleotides for the PAM site can differ depending on the type of the CRISPR effector protein, e.g., Cas enzyme. The protospacer can be referred to as a target site (or a genomic target site). The CRISPR polynucleotide can pair with (or hybridize to) the opposite strand of the protospacer (binding site) to direct the Cas enzyme to the target region.

### A. CRISPR Complex Overview

A CRISPR complex can be a non-naturally occurring or engineered DNA or RNA-targeting system comprising one or more DNA or RNA-targeting CRISPR effector proteins and one or more CRISPR polynucleotides. The one or more CRISPR polynucleotides can be any CRISPR polynucleotide provided herein. The target sequence can be a sequence to which a guide sequence of a CRISPR polynucleotide is designed to have complementarity, and “complementarity” can refer to the ability of a nucleic acid to form hydrogen bond(s) with another nucleic acid sequence by either traditional Watson-Crick base-pairing or other non-traditional types of base-paring. The CRISPR complex can interact with two nucleic acid strands that form a duplex structure, three or more strands forming a multi stranded complex, a single self-hybridizing strand, or any combination of these.

Upon binding of the CRISPR complex to the target sequence, sequences associated with the target sequence can be modified by the CRISPR effector protein. The CRISPR effector protein can be part of a fusion protein that can comprise one or more heterologous protein domains (e.g. about or more than about 1, 2, 3, 4, 5, 6, 7, 8, 9, 10, or more domains in addition to the CRISPR effector protein). In some examples, the functionality of the CRISPR complex is conferred by the heterologous protein domains.

In some cases, one or more elements of a CRISPR system can be derived from a type I, type II, or type III CRISPR system. In the CRISPR type II system, the CRISPR polynucleotide (e.g., guide RNA) can interact with Cas endonuclease and direct the nuclease activity of the Cas enzyme to a target region. The target region can comprise a “protospacer” and a “protospacer adjacent motif” (PAM), and both domains can be used for a Cas enzyme mediated activity (e.g., cleavage). The guide sequence can pair with (or hybridize) the opposite strand of the protospacer (binding site) to direct the Cas enzyme to the target region. The PAM site can refer to a short sequence recognized by the Cas enzyme and, in some cases, required for the Cas enzyme activity. The sequence and number of nucleotides for the PAM site can differ depending on the type of the Cas enzyme.

The CRISPR/Cas complex (CRISPR system) can be any one two classes. Class 1 can use a system of multiple Cas proteins to degrade foreign nucleic acids. Class 2 systems can use a single Cas protein for the same purpose. Class 1 can be divided into types I, III, and IV; class 2 can be divided into types II, V and VI. One or more elements of a CRISPR system can be derived from a type I, type II, or type III CRISPR/Cas system. In the CRISPR type II effector protein, the guide polynucleotide (e.g. RNA) can interact with the CRISPR effector protein (e.g., Cas) and direct the nuclease activity of the Cas enzyme to a target region. Type II Cas proteins include Cas9, Type V includes Cas12 (Cpf1), and Type VI includes Cas13 and Cas 14. The canonical target of Type II and Type V can be RNA whereas the canonical target of Type V can be DNA.

### B. CRISPR Effector Protein

A CRISPR effector protein can comprise a Cas protein of, or derived from, a CRISPR/Cas type I, type II, or type III system, which can have an RNA-guided polynucleotide-binding or nuclease activity. Examples of suitable Cas proteins include CasX, Cas3, Cas4, Cas5, Cas5e (or CasD), Cas6, Cas6e, Cas6f, Cas7, Cas8a1, Cas8a2, Cas8b, Cas8c, Cas9 (also known as Csn1 and Csxl2), Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, Cu1966, homologues thereof, and modified versions thereof. In some cases, a Cas protein can comprise a protein of or derived from a CRISPR/Cas type V or type VI system, such as Cpf1, C2c1, C2c2, homologues thereof, and modified versions thereof. In some cases, a CRISPR effector protein can be a catalytically dead or inactive Cas (dCas) protein. The Cas protein can be a Type II Cas9 from Streptococcus pyogenes (SpCas9), Neisseria meningitidis (NmCas9), Methanococcus maripaludis, Corynebacterium diphtheriae, Corynebacterium efficiens, Corynebacterium glutamicum, Corynebacterium kroppenstedtii, Mycobacterium abscessus, Nocardia farcinica, Rhodococcus erythropolis, Rhodococcus jostii, Rhodococcus opacus, Acidothermus cellulolyticus, Arthrobacter chlorophenolicus, Kribbella flavida, Thermomonospora curvata, Bifidobacterium dentium, Bifidobacterium longum, Slackia heliotrinireducens, Persephonella marina, Bacteroides fragilis, Capnocytophaga ochracea, Flavobacterium psychrophilum, Akkermansia muciniphila, Roseijlexus castenholzii, Roseijlexus, Synechocystis, Elusimicrobium minutum, Fibrobacter succinogenes, Bacillus cereus, Listeria innocua, Lactobacillus casei, Lactobacillus rhamnosus, Lactobacillus salivarius, Streptococcus agalactiae, Streptococcus dysgalactiae, Streptococcus equi, Streptococcus gallolyticus, Streptococcus gordonii, Streptococcus mutans, Streptococcus thermophilus, Clostridium botulinum, Clostridium cellulolyticum, Finegoldia magna, Eubacterium rectale, Mycoplasma gallisepticum, Mycoplasma mobile, Mycoplasma penetrans, Mycoplasma synoviae, Streptobacillus moniliformis, Bradyrhizobium, Nitrobacter hamburgensis, Rhodopseudomonas palustris, Parvibaculum lavamentivorans, Dinoroseobacter shibae, Gluconacetobacter diazotrophicus, Azospirillum, Rhodospirillum rubrum, Acidovorax ebreus, Verminephrobacter eiseniae, Desulfovibrio salexigens, Campylobacter jejuni, Campylobacter lari, Helicobacter hepaticus, Wolinella succinogenes, Tolumonas auensis, Pseudoalteromonas atlantica, Shewanella pealeana, Legionella pneumophila, Actinobacillus succinogenes, Pasteurella multocida, Francisella novicida, Francisella tularensis, or Treponema denticola.

The Cas protein can be a Type I Cas7 or Cas 1 from Aeropyrum pernix, Desulfurococcus kamchatkensis, Ignicoccus hospitalis, Staphylothermus marinus, Hyperthermus butylicus, Metallosphaera sedula, Sulfolobus islandicus, Sulfolobus solfataricus, Sulfolobus tokodaii, Thermofilum pendens, Caldivirga maquilingensis, Pyrobaculum aerophilum, Pyrobaculum arsenaticum, Pyrobaculum calidifontis, Thermoproteus neutrophilus, Archaeoglobus fulgidus, Ferroglobus placidus, Haloarcula marismortui, Halomicrobium mukohataei, Halorhabdus utahensis, Halorubrum lacusprofundi, Natronomonas pharaonis, Methanobrevibacter ruminantium, Methanobrevibacter smithii, Methanosphaera stadtmanae, Methanothermobacter thermautotrophicus, Methanocaldococcus fervens, Methanocaldococcus jannaschii, Methanocaldococcus, Methanocaldococcus vulcanius, Methanococcus aeolicus, Methanococcus maripaludis, Methanococcus vannielii, Methanocorpusculum labreanum, Methanospirillum hungatei, Methanosphaerula palustris, Methanosaeta thermophila, Methanococcoides burtonii, Methanosarcina acetivorans, Methanosarcina barkeri, Methanosarcina mazei, Pyrococcus abyssi, Pyrococcus furiosus, Pyrococcus horikoshii, Thermococcus gammatolerans, Thermococcus kodakarensis, Thermococcus sibiricus, Picrophilus torridus, Candidatus Korarchaeum cryptofilum, Nanoarchaeum equitans, Acidimicrobium ferrooxidans, Catenulispora acidiphila, Corynebacterium aurimucosum, Corynebacterium diphtheriae, Corynebacterium glutamicum, Corynebacterium jeikeium, Corynebacterium urealyticum, Nocardia farcinica, Rhodococcus erythropolis, Frankia alni, Frankia, Nakamurella multipartita, Rothia mucilaginosa, Xylanimonas cellulosilytica, Salinispora arenicola, Salinispora tropica, Actinosynnema mirum, Saccharomonospora viridis, Streptomyces avermitilis, Streptomyces griseus, Thermobifida fusca, Thermomonospora curvata, Bifidobacterium adolescentis, Bifidobacterium animalis, Bifidobacterium dentium, Gardnerella vaginalis, Eggerthella lenta, Rubrobacter xylanophilus, Aquifex aeolicus, Hydrogenobacter thermophilus, Hydrogenobaculum, Thermocrinis albus, Persephonella marina, Sulfurihydrogenibium azorense, Sulfurihydrogenibium, Bacteroides fragilis, Parabacteroides distasonis, Porphyromonas gingivalis, Spirosoma linguale, Rhodothermus marinus, Chlorobaculum tepidum, Chlorobium chlorochromatii, Chlorobium limicola, Chlorobium phaeobacteroides, Chlorobium phaeovibrioides, Pelodictyon luteolum, Pelodictyon phaeoclathratiforme, Chloroherpeton thalassium, Prosthecochloris aestuarii, Chloroflexus aggregans, Chloroflexus aurantiacus, Chloroflexus, Roseiflexus castenholzii, Roseiflexus, Herpetosiphon aurantiacus, Dehalococcoides, Sphaerobacter thermophilus, Thermomicrobium roseum, Cyanothece, Microcystis aeruginosa, Synechococcus, Synechocystis, Anabaena variabilis, Nostoc punctiforme, Nostoc, Deinococcus geothermalis, Thermus thermophilus, Dictyoglomus thermophilum, Dictyoglomus turgidum, Acidobacterium capsulatum, Alicyclobacillus acidocaldarius, Anoxybacillus flavithermus, Bacillus cytotoxicus, Bacillus clausii, Bacillus halodurans, Geobacillus, Lysinibacillus sphaericus, Exiguobacterium sibiricum, Listeria monocytogenes, Listeria seeligeri, Lactobacillus casei, Lactobacillus delbrueckii, Lactobacillus fermentum, Lactobacillus helveticus, Streptococcus equi, Streptococcus mutans, Streptococcus pyogenes, Alkaliphilus metalliredigens, Clostridium botulinum, Clostridium cellulolyticum, Clostridium difficile, Clostridium kluyveri, Clostridium novyi, Clostridium perfringens, Clostridium tetani, Clostridium thermocellum, Finegoldia magna, Symbiobacterium thermophilum, Eubacterium rectale, Heliobacterium modesticaldum, Candidatus Desulforudis audaxviator, Desulfitobacterium hafniense, Desulfotomaculum acetoxidans, Desulfotomaculum reducens, Pelotomaculum thermopropionicum, Syntrophomonas wolfei, Anaerocellum thermophilum, Acidaminococcus fermentans, Halothermothrix orenii, Carboxydothermus hydrogenoformans, Ammonifex degensii, Moorella thermoacetica, Thermoanaerobacter italicus, Thermoanaerobacter pseudethanolicus, Thermoanaerobacter, Thermoanaerobacter tengcongensis, Caldicellulosiruptor saccharolyticus, Fusobacterium nucleatum, Leptotrichia buccalis, Thermodesulfovibrio yellowstonii, Nitrobacter winogradskyi, Methylobacterium nodulans, Methylobacterium, Dinoroseobacter shibae, Rhodobacter sphaeroides, Acetobacter pasteurianus, Acidiphilium cryptum, Gluconacetobacter diazotrophicus, Granulibacter bethesdensis, Azospirillum, Rhodospirillum centenum, Rhodospirillum rubrum, Zymomonas mobilis, Acidovorax citrulli, Acidovorax, Delftia acidovorans, Rhodoferax ferrireducens, Verminephrobacter eiseniae, Leptothrix cholodnii, Methylobacillus flagellatus, Chromobacterium violaceum, Laribacter hongkongensis, Neisseria gonorrhoeae, Nitrosomonas europaea, Nitrosomonas eutropha, Aromatoleum aromaticum, Thauera, Candidatus Accumulibacter phosphatis, Desulfatibacillum alkenivorans, Desulfobacterium autotrophicum, Desulfococcus oleovorans, Desulfotalea psychrophila, Desulfohalobium retbaense, Desulfovibrio desulfuricans, Desulfovibrio magneticus, Desulfovibrio vulgaris, Geobacter bemidjiensis, Geobacter lovleyi, Geobacter metallireducens, Geobacter, Geobacter sulfurreducens, Geobacter uraniireducens, Pelobacter carbinolicus, Pelobacter propionicus, Anaeromyxobacter dehalogenans, Anaeromyxobacter, Myxococcus xanthus, Haliangium ochraceum, Sorangium cellulosum, Syntrophus aciditrophicus, Syntrophobacter fumaroxidans, Campylobacter concisus, Campylobacter curvus, Campylobacter fetus, Campylobacter hominis, Sulfurospirillum deleyianum, Helicobacter pylori, Tolumonas auensis, Alteromonas macleodii, Teredinibacter turnerae, Psychromonas ingrahamii, Shewanella baltica, Shewanella oneidensis, Shewanella piezotolerans, Shewanella putrefaciens, Shewanella, Dichelobacter nodosus, Allochromatium vinosum, Nitrosococcus oceani, Alkalilimnicola ehrlichii, Thioalkalivibrio, Halothiobacillus neapolitanus, Citrobacter rodentium, Cronobacter sakazakii, Cronobacter turicensis, Dickeya dadantii, Dickeya zeae, Enterobacter, Erwinia pyrifoliae, Erwinia tasmaniensis, Escherichia coli, Escherichia fergusonii, Klebsiella pneumoniae, Klebsiella variicola, Pectobacterium atrosepticum, Pectobacterium wasabiae, Photorhabdus, Photorhabdus luminescens, Salmonella enterica, Shigella boydii, Shigella flexneri, Shigella sonnei, Xenorhabdus bovienii, Yersinia pestis, Yersinia pseudotuberculosis, Coxiella burnetii, Legionella pneumophila, Methylococcus capsulatus, Hahella chejuensis, Chromohalobacter salexigens, Marinomonas, Actinobacillus pleuropneumoniae, Actinobacillus succinogenes, Aggregatibacter actinomycetemcomitans, Aggregatibacter aphrophilus, Mannheimia succiniciproducens, Pasteurella multocida, Acinetobacter baumannii, Acinetobacter, Azotobacter vinelandii, Cellvibrio japonicus, Pseudomonas aeruginosa, Pseudomonas mendocina, Pseudomonas stutzeri, Vibrio fischeri, Photobacterium profundum, Vibrio cholerae, Vibrio harveyi, Vibrio parahaemolyticus, Xanthomonas, Xanthomonas axonopodis, Xanthomonas oryzae, Magnetococcus, Leptospira borgpetersenii, Leptospira interrogans, Fervidobacterium nodosum, Kosmotoga olearia, Petrotoga mobilis, Thermosipho africanus, Thermosipho melanesiensis, Thermotoga lettingae, Thermotoga maritima, Thermotoga neapolitana, Thermotoga petrophila, Thermotoga, or Thermobaculum terrenum.

The Cas protein can be Type III Cas10 from Desulfurococcus kamchatkensis, Ignicoccus hospitalis, Staphylothermus marinus, Hyperthermus butylicus, Metallosphaera sedula, Sulfolobus acidocaldarius, Sulfolobus islandicus, Sulfolobus solfataricus, Sulfolobus tokodaii, Thermofilum pendens, Caldivirga maquilingensis, Pyrobaculum aerophilum, Pyrobaculum arsenaticum, Pyrobaculum calidifontis, Pyrobaculum islandicum, Thermoproteus neutrophilus, Archaeoglobus fulgidus, Natronomonas pharaonis, Methanobrevibacter ruminantium, Methanosphaera stadtmanae, Methanothermobacter thermautotrophicus, Methanocaldococcus fervens, Methanocaldococcus jannaschii, Methanocaldococcus, Methanocaldococcus vulcanius, Methanococcus aeolicus, Methanococcus vannielii, Methanospirillum hungatei, Methanosaeta thermophila, Methanosarcina acetivorans, Methanosarcina barkeri, Methanosarcina mazei, Methanopyrus kandleri, Pyrococcus furiosus, Pyrococcus horikoshii, Thermococcus onnurineus, Picrophilus torridus, Thermoplasma volcanium, Aciduliprofundum boonei, Candidatus Korarchaeum cryptofilum, Mycobacterium bovis, Mycobacterium tuberculosis, Frankia, Salinispora tropica, Saccharomonospora viridis, Saccharopolyspora erythraea, Thermobifida fusca, Rubrobacter xylanophilus, Aquifex aeolicus, Thermocrinis albus, Sulfurihydrogenibium azorense, Sulfurihydrogenibium, Porphyromonas gingivalis, Rhodothermus marinus, Chlorobaculum parvum, Chlorobium phaeobacteroides, Chlorobium phaeobacteroides, Pelodictyon phaeoclathratiforme, Chloroherpeton thalassium, Methylacidiphilum infernorum, Chloroflexus aggregans, Chloroflexus aurantiacus, Chloroflexus, Roseiflexus castenholzii, Roseiflexus, Herpetosiphon aurantiacus, Thermomicrobium roseum, Cyanothece, Microcystis aeruginosa, Synechococcus, Synechocystis, Anabaena variabilis, Nostoc punctiforme, Nostoc, Deinococcus geothermalis, Thermus thermophilus, Dictyoglomus thermophilum, Dictyoglomus turgidum, Candidatus Solibacter usitatus, Fibrobacter succinogenes, Alicyclobacillus acidocaldarius, Bacillus halodurans, Geobacillus, Staphylococcus epidermidis, Staphylococcus lugdunensis, Streptococcus sanguinis, Streptococcus thermophilus, Clostridium botulinum, Clostridium tetani, Clostridium thermocellum, Candidatus Desulforudis audaxviator, Desulfotomaculum acetoxidans, Desulfotomaculum reducens, Pelotomaculum thermopropionicum, Syntrophomonas wolfei, Anaerocellum thermophilum, Veillonella parvula, Halothermothrix orenii, Carboxydothermus hydrogenoformans, Ammonifex degensii, Thermoanaerobacter italicus, Thermoanaerobacter pseudethanolicus, Thermoanaerobacter, Thermoanaerobacter tengcongensis, Caldicellulosiruptor saccharolyticus, Ureaplasma parvum, Leptotrichia buccalis, Streptobacillus moniliformis, Thermodesulfovibrio yellowstonii, Pirellula staleyi, Rhodospirillum centenum, Rhodospirillum rubrum, Nitrosomonas europaea, Nitrosomonas eutropha, Candidatus Accumulibacter phosphatis, Desulfococcus oleovorans, Myxococcus xanthus, Haliangium ochraceum, Sorangium cellulosum, Syntrophus aciditrophicus, Syntrophobacter fumaroxidans, Arcobacter butzleri, Campylobacter fetus, Teredinibacter turnerae, Allochromatium vinosum, Halorhodospira halophila, Thioalkalivibrio, Dickeya dadantii, Pectobacterium carotovorum, Marinomonas, Mannheimia succiniciproducens, Vibrio vulnificus, Fervidobacterium nodosum, Kosmotoga olearia, Thermosipho africanus, Thermosipho melanesiensis, Thermotoga maritima, Thermotoga naphthophila, Thermotoga neapolitana, Thermotoga petrophila, Thermotoga, or Thermobaculum terrenum.

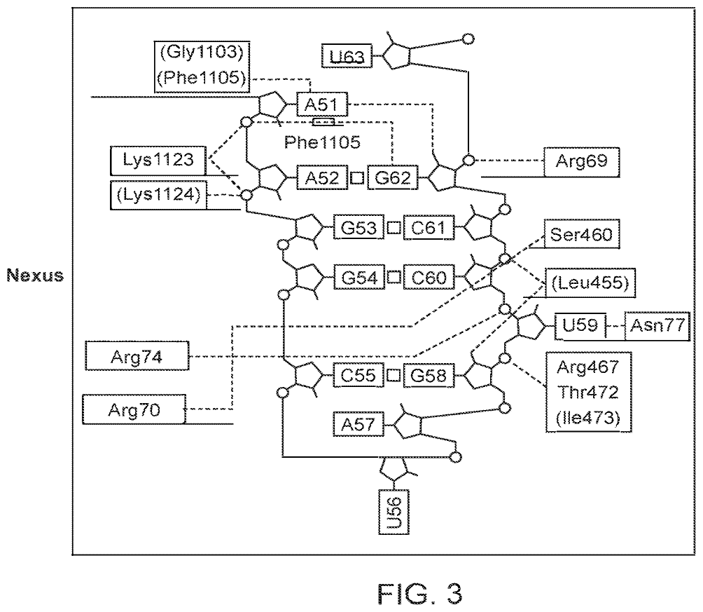

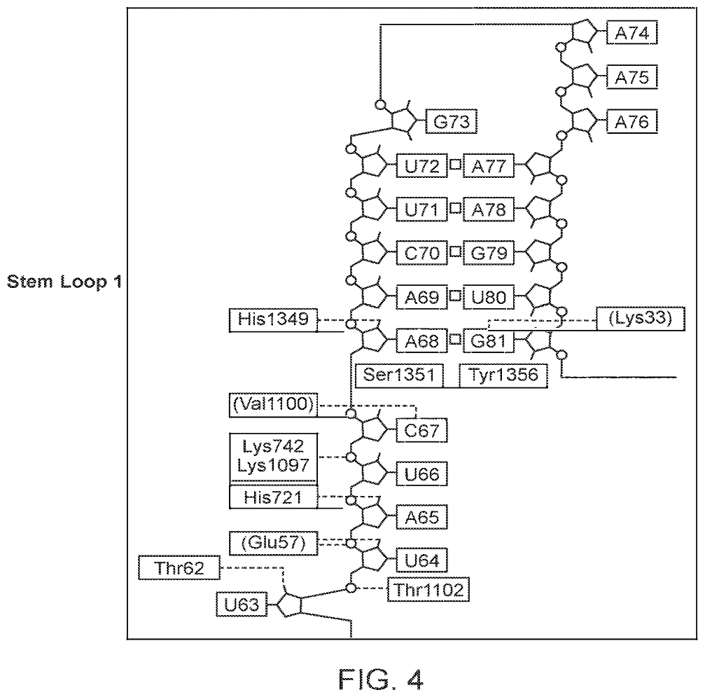

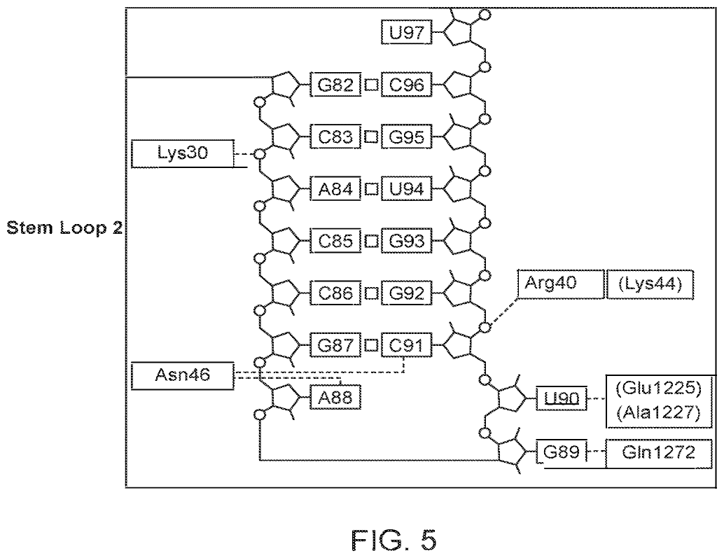

The Cas protein can be Cas9. Cas9 can comprise an alpha helical lobe and a nuclease lobe. The alpha helical lobe can comprise three regions, along a helix referred to as the bridge helix, a REC1 domain, and a REC2 domain. The nuclease lobe can comprise a RuvC domain, a HNH domain and a PAM-interacting domain. FIG. 3 highlights amino acids of different domains in close proximity with the nexus which can be conjugating sites such as the REC1 domain (Ser460, Leu455, Arg467, Thr472, Ile 473), bridge helix (Arg69, Asn77, Arg74, Arg70), and PAM-interacting domain (Gly1103, Phe1105, Lys1123, Lys1124, Phe1105). FIG. 4 highlights amino acids of different domains in close proximity with stem loop 1 which can be conjugating sites such as the RuvC domain (Lys33, Lys742, Lys1097, His721, Glu57), PAM-interacting domain (Ser1351, Tyr1356, His1349, Val1100, Thr1102), and bridge helix domain (Thr62). FIG. 5 highlights amino acids of different domains in close proximity with stem loop 2 which can be conjugating sites such as the RuvC domain (Lys30, Asn46, Arg40, Lys44) and PAM-interacting domain (Glu1225, Ala1227, Gln1272).

Upon nucleic acid binding with a CRISPR polynucleotide (e.g., RNA) and a target DNA molecule the nuclease lobe can rotate ˜100° relative to the alpha helical lobe. One or more crosslinking groups can be located so as to retain the full activity of the CRISPR effector protein, and the crosslinking method can permit retention of the full activity of the CRISPR effector protein (e.g., Cas nuclease).

### C. Polynucleotides for Use in CRISPR Complexes

The CRISPR polynucleotide can comprise RNA, DNA-RNA hybrids, or derivatives thereof. The CRISPR polynucleotide can comprise nucleosides, which can comprise a base covalently attached to a sugar moiety, e.g., ribose or deoxyribose. The nucleosides can be ribonucleosides or deoxyribonucleosides. The nucleosides can comprise bases linked to amino acids or amino acid analogs, which can comprise free carboxyl groups, free amino groups, or protecting groups. The protecting groups can be a protecting group described, e.g., in P. G. M. Wuts and T. W. Greene, “Protective Groups in Organic Synthesis”, 2nd Ed., Wiley-Interscience, New York, 1999. The CRISPR polynucleotides can comprise a canonical cyclic nucleotide, e.g., cAMP, cGMP, cCMP, cUMP, cIMP, cXMP, or cTMP. A canonical nucleotide base can be adenine, cytosine, uracil, guanine, or thymine. The nucleotide can comprise a nucleoside attached to a phosphate group or a phosphate analog.

The CRISPR polynucleotide can exist as one or more molecules of RNA, or DNA (e.g., in one or more vectors encoding said one or more molecules of RNA or protein). The CRISPR polynucleotides can be deoxyribonucleic acids (DNA), ribonucleic acids (RNA) and polymers thereof in either single-, double- or multi-stranded form. The CRISPR polynucleotide can comprise single-, double- or multi-stranded DNA or RNA, genomic DNA, cDNA, DNA-RNA hybrids, or a polymer comprising purine and/or pyrimidine bases or other natural, chemically modified, biochemically modified, non-natural, synthetic or derivatized nucleotide bases.

The polynucleotide (CRISPR polynucleotide) sequence used in the CRISPR-Cas system can comprise a crRNA sequence and a tracrRNA sequence. In nature, crRNA and tracrRNA can exist as two separate RNA molecules. The term “tracrRNA” or “tracrRNA segment,” can refer to a polynucleotide molecule or portion thereof that includes a protein-binding segment (e.g., the protein-binding segment is capable of interacting with a CRISPR-effector protein, such as a Cas9). The terms “guide RNA” and “gRNA” can encompass a single guide RNA (sgRNA), where the crRNA segment and the tracrRNA segment are located in the same RNA molecule.

In some cases, the gRNA can be a complex (e.g., via hydrogen bonds) of a CRISPR RNA (crRNA) segment and a trans-activating crRNA (tracrRNA) segment. The crRNA can comprise a hybridizing polynucleotide sequence and a tracrRNA-binding polynucleotide sequence. The hybridizing polynucleotide sequence can hybridize to a portion of a target nucleic acid (e.g., a selected exon). The hybridizing polynucleotide sequence of the crRNA can range from 17 to 23 nucleotides. The hybridizing polynucleotide sequence of the crRNA can be at least 17, 18, 19, 20, 21, 22, 23, or more nucleotides. The hybridizing polynucleotide sequence of the crRNA can be at most 23, 22, 21, 20, 19, 18, 17, or less nucleotides. In an example, the hybridizing polynucleotide sequence of the crRNA is 20 nucleotides. The hybridizing polynucleotide can be a guide sequence. The guide sequence can comprise sufficient complementarity with a target nucleic acid sequence to hybridize with the target nucleic acid sequence. The degree of complementarity, when optimally aligned using a suitable alignment algorithm, can be about or more than about 50%, 60%, 75%, 80%, 85%, 90%, 95%, 97.5%, or 99%. The degree of complementarity can be 100%. In some cases, the guide sequence e.g., can be about 5, 6, 7, 8, 9, 10, 11, 12, 13, 14, 15, 16, 17, 18, 19, 20, 25, 30, 40, 50, or more nucleotides in length. The guide sequence can be about 5 to about 40 nucleotides in length. The guide sequence can be designed in a way that reduces the likelihood that the guide sequence base pairs to itself or base pairs with another portion of the CRISPR polynucleotide. About or less than about 75%, 50%, 40%, 30%, 25%, 20%, 15%, 10%, 5%, 1%, or fewer of the nucleotides of the guide sequence can form a base-pair with another portion of the guide sequence or another portion of the CRISPR polynucleotide when the CRISPR polynucleotide is optimally folded.

In some cases, a single CRISPR polynucleotide is crosslinked to a single CRISPR effector protein. The single CRISPR polynucleotide can comprise a guide sequence and sequence that crosslinks to the CRISPR effector protein. The sequence that can crosslink the CRISPR effector protein can be a trans-activating RNA (tracrRNA). When a single CRISPR polynucleotide comprises a guide sequence and a tracrRNA, the single CRISPR polynucleotide can be referred to as a single guide RNA (or sgRNA).

In some cases, two CRISPR polynucleotides may be crosslinked to a single CRISPR effector protein. A first CRISPR polynucleotide can comprise a guide sequence, and a second CRISPR polynucleotide can comprise a tracrRNA and lack a guide sequence.

In some cases, the first CRISPR polynucleotide comprises a guide sequence and a first part of the sequence (which can be referred to as a tracr mate sequence) that forms the crRNA, and the second CRISPR polynucleotide comprises a second part of the sequence that forms the tracrRNA (which can be referred to as the tracr sequence). In some cases, the tracr sequence (or tracrRNA) hybridizes to the ‘tracr mate’ sequence within the crRNA thereby forming a double-stranded RNA duplex protein binding segment recognized by the CRISPR effector protein. A CRISPR polynucleotide comprising a guide sequence (also known as spacer sequence) but lacking sequence that can bind to the CRISPR effector protein can be referred to as a guide RNA (or gRNA). A CRISPR polynucleotide comprising a guide sequence and only part of a sequence that can bind to the CRISPR effector protein (e.g., a tracr mate sequence) (and lacks a tracr sequence) can also be referred to as a guide RNA (or gRNA) or crRNA.

A tracrRNA can hybridize to the ‘tracr mate’ sequence within the crRNA thereby forming a double-stranded RNA duplex protein binding segment recognized by the CRISPR effector protein. In some examples, the hybridization between the two produces a secondary structure, such as a hairpin. In some cases, the CRISPR polynucleotide sequence can comprise three, four, five, or more hairpins. The tracrRNA can comprise, or consist of, one or more hairpins and can be at least 10, 20, 30, 40, 50, 60, 70, 80, 90, or 100 nucleotides in length.

In some cases, a first CRISPR polynucleotide can be crRNA and a second CRISPR polynucleotide can be tracrRNA and the first CRISPR polynucleotide and second CRISPR polynucleotide can be two separate RNA molecules. In some cases, a single CRISPR polynucleotide can comprise (1) a guide sequence (or crRNA comprising a guide sequence) capable of hybridizing to a target sequence (e.g., a genomic target locus in a eukaryotic cell) and (2) a tracrRNA. In some cases, the first CRISPR polynucleotide can comprise (1) a guide sequence (or crRNA comprising a guide sequence) (e.g., capable of hybridizing to a target sequence in the eukaryotic cell); and (2) a tracr mate sequence (also known as direct repeat sequence) but lacking a tracrRNA sequence. The CRISPR effector protein can associate with a guide sequence capable of hybridizing to a target sequence and a tracr mate sequence (direct repeat sequence), without the requirement for a tracrRNA.

When the tracr and tracr mate sequences are in a single CRISPR polynucleotide, the tracr and tracr mate sequences can be covalently linked. The tracr and tracr mate sequence can be linked through a phosphodiester bond. The tracr and tracr mate can be covalently linked via a non-nucleotide loop comprising a moiety such as a spacer, attachment, bioconjugate, chromophore, reporter group, dye labeled RNA, or non-naturally occurring nucleotide analogue. The spacer can be a polyether (e.g., polyethylene glycol, polyalcohol, polypropylene glycol or mixtures of ethylene and propylene glycol), polyamine group (e.g., spennine, spermidine, or a polymeric derivative thereof), polyester (e.g., poly(ethyl acrylate)), polyphosphodiester, alkylene, and combinations thereof. The attachment can be a fluorescent label. The bioconjugate can be, e.g., a peptide, a glycoside, a lipid, a cholesterol, a phospholipid, a diacyl glycerol, a dialkyl glycerol, a fatty acid, a hydrocarbon, an enzyme substrate, a steroid, biotin, digoxigenin, a carbohydrate, or a polysaccharide. The chromophore, reporter group, or dye-labeled RNA can be a fluorescent dye, e.g., fluorescein or rhodamine, a chemiluminescent, an electrochemiluminescent, or a bioluminescent marker compound.

Overall, the crRNA can range from 35 to 45 nucleotides. The crRNA can be at least 35, 36, 37, 38, 39, 40, 41, 42, 43, 44, 45, or more nucleotides. The crRNA can be at most 45, 44, 43, 42, 41, 40, 39, or less nucleotides. The tracrRNA can range from 60 to 80 nucleotides. The tracrRNA can be at least 60, 61, 62, 63, 64, 66, 68, 70, 72, 74, 76, 78, 80, or more nucleotides. The tracrRNA can be at most 80, 79, 78, 77, 76, 74, 72, 70, 68, 66, 64, 62, 60, or less nucleotides. In an example, the tracrRNA can be 72 nucleotides. In another example, the hybridizing polynucleotide sequence of the crRNA is 20 nucleotides, the crRNA is 42 nucleotides, and the respective tracrRNA is 72 nucleotides. In another example, the hybridizing polynucleotide of the crRNA is 20 nucleotides, the crRNA is a total of 34 nucleotides, and the respective tracrRNA is 66 nucleotides.

In some instances, the crRNA and tracrRNA are joined into a single guide RNA molecule called a sgRNA, or “single guide RNA.” Each sgRNA can comprise a constant region from about 20 to about 30, about 30 to about 40, about 40 to about 50, about 50 to about 60, about 60 to about 70, about 70 to about 80, or about 80 to about 100 nucleotides in length. Each sgRNA can comprise at least 90, 91, 92, 93, 94, 95, 96, 97, 98, 99, 100, 101, 102, 103, 104, 105, 106, 107, 108, 109, or 110 nucleotides.

Alternatively, the gRNA can be a complex of three or more RNA chains. At least one RNA chain of the complex of three or more RNA chains can comprise a hybridizing polynucleotide sequence. At least one RNA chain of the complex of three or more RNA chains can comprise a CRISPR effector protein (e.g., Cas enzyme) binding sequence.

When the gRNA hybridizes to a target nucleic acid molecule, the hybridized portion of the gene can be a target region (or target locus) that comprises a protospacer (target site), a protospacer adjacent motif (PAM) that is recognized by the CRISPR effector protein (e.g., Cas enzyme), and the opposite strand of the protospacer (binding site). The opposite strand of the protospacer can be the gRNA-hybridizing genomic region (sequence). The gRNA-hybridizing sequence in the target nucleic acid sequence can range from 17 to 23 nucleotides. The gRNA-hybridizing sequence in the gene can be at least 17, 18, 19, 20, 21, 22, 23, or more nucleotides. The gRNA-hybridizing sequence in the gene can be at most 23, 22, 21, 20, 19, 18, 17, or less nucleotides.

The CRISPR effector protein (e.g., Cas protein) can be Cas9, wherein the tracrRNA can interact with the alpha-helical lobe and the nuclease lobe of Cas9 through four hairpin loops; two hairpin loops can interact with each lobe respectively. The crRNA can be designed to complementarily bind to a target nucleic acid (e.g., DNA) sequence. A full length sgRNA target DNA binding region can be 20 nucleotides for Cas9. For Cas9, PAM sequences can include 3′-NGG, 3′-NGGNG (SEQ ID NO: 1), 3′NNAGAAW (SEQ ID NO: 2), and 3′-ACAY (SEQ ID NO: 3) where N is any nucleotide, W is A or T, and Y is C or T.

In some cases, to increase the effectiveness of a CRISPR polynucleotide, e.g., gRNA or sgRNA, other secondary structures may be added to the CRISPR polynucleotide, e.g., gRNA or sgRNA to enhance the stability of the CRISPR polynucleotide. In some cases, the increased stability can improve nucleic acid editing.

### III. Stabilized CRISPR Complexes

The present disclosure encompasses a CRISPR effector protein covalently bound to a sgRNA, which can be termed a “locked CRISPR complex.” The present disclosure encompasses a CRISPR effector protein covalently bound to a separate crRNA and/or a tracrRNA. Also provided herein are CRISPR complexes in which sgRNA and the CRISPR effector protein have enhanced binding affinity. A CRISPR polynucleotide can comprise gRNA, sgRNA, crRNA, or tracrRNA. A CRISPR effector protein can be covalently bound (e.g., crosslinked) to any CRISPR polynucleotide described herein, e.g., a gRNA, sgRNA, crRNA, tracrRNA, a CRISPR ON polynucleotide, a CRISPR OFF polynucleotide, a CRISPR ON/OFF polynucleotide, or a CRISPR polynucleotide modified to decrease off-target editing.

The CRISPR-Cas system can be modified to both knock out specific genes as well as knock-in specific genes. CRISPR-mediated knockouts can be generated through the non-homologous end joining repair pathway of a cell. In this event, CRISPR-Cas can bind to a target nucleic acid (e.g., DNA) region complementary to the bound RNA and execute a double strand cut in the target nucleic acid (e.g., DNA) region. When designing the sgRNA, a unique 3-9 nucleotide PAM recognition region can be designed in the sgRNA near the target nucleic acid (e.g., DNA) for those that Cas nucleases which require a PAM recognition site. Not every Cas nuclease requires a PAM region; for instance, Cas 14a does not require a PAM region for identification.

Enhancing the stability of a sgRNA in complex with a CRISPR effector protein by at least one covalent bond can decrease the number of off-target cleavage events, lowering the cell toxicity as compared to a CRISPR complex that is not covalently bound. CRISPR effector proteins crosslinked to a sgRNA (“locked”) can be used to reduce off target editing as compared to Cas9 complexed with a standard sgRNA. Off-target editing can be determined using ICE (Inference of CRISPR Editing) measured the amount of gene editing by analyzing Sanger sequencing traces and mapping level of sequence breakdown to determine indel formation frequencies, as described in Hsiau et al. “Inference of CRISPR Edits from Sanger Trace Data”, Jan. 14, 2019 bioRxiv or deep-sequencing techniques as described in Tsai et al. “GUIDE-seq enables genome-wide profiling of off-target cleavage by CRISPR-Cas nucleases”, Nature Biotechnology 33, 187-197 (2015).

Covalently locking the sgRNA to the CRISPR effector protein can decrease the probability that sgRNA will cause toxicity within a cell. Locking a sgRNA to a CRISPR effector protein can increase the accuracy of dosing, by allowing one to administer a single species of CRISPR complex with a guide RNA designed for a specific target. One can administer two or more locked CRISPR complexes with unique targets from one another for a complex therapy targeting multiple sites. Additionally, formulating a sgRNA sequence in complex with a CRISPR effector protein such that it cannot dissociate from the complexed state may grant greater protection from degredation both in formulation and after administration.

### A. Modified CRISPR Polynucleotide

Disclosed herein is a CRISPR polynucleotide modified with at least one unnatural nucleotide for conjugating (e.g., via cross-linking) and a sequence for modulating activity. The sequence for modifying activity can be a CRISPR ON polynucleotide sequence, and CRISPR OFF polynucleotide sequence, a CRISPR ON/OFF polynucleotide sequence, or a CRISPR nucleotide modified to lower off-target editing. The unnatural nucleotide can be located in the tracrRNA, the crRNA, or the guide sequence of the crRNA.

In some cases, the CRISPR polynucleotide can be modified to improve the CRISPR polynucleotide's resistance to nucleases, serum stability, target specificity, blood system circulation, tissue distribution, tissue penetration, cellular uptake, potency, and/or cell permeability. For example, certain CRISPR polynucleotide modifications can increase nuclease stability, and/or lower interferon induction, without significantly affecting activity of the CRISPR polynucleotide (e.g., sgRNA). The modified CRISPR polynucleotide can have improved stability in serum and/or cerebral spinal fluid compared to an unmodified CRISPR polynucleotide having the same sequence. The CRISPR polynucleotide (e.g., sgRNA) disclosed herein can comprise one or more modifications at various locations, including at a sugar moiety, a phosphodiester linkage, and/or a base. A modified CRISPR polynucleotide as described herein can include both a sgRNA and a separate crRNA and tracrRNA. A CRISPR polynucleotide can be bound to a CRISPR effector protein by hydrogen bonding interactions.

Provided herein are CRISPR polynucleotides that can be conjugated to a CRISPR effector protein by one or more covalent bonds, forming a locked CRISPR complex. There can be 1 to 3, 3 to 6, 6 to 9, 9 to 12, 12 to 15, 15 to 18, 18 to 21, 21 to 24, 24 to 27, 27 to 30, 30 to 33, 33 to 36, 36 to 39, 39 to 42, 42 to 45, 45 to 48, 48 to 51, 51 to 54, 54 to 57, 57 to 60, 60 to 62, 62 to 65, 65 to 68, 68 to 71, 71 to 74, 74 to 77, 77 to 80, 80 to 83, 83 to 86, 86 to 89, 89 to 91, 91 to 94, 94 to 97, 97 to 100 covalent bonds between a CRISPR polynucleotide and a CRISPR effector protein. There can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 covalent bonds between a CRISPR polynucleotide and a CRISPR effector protein. There can be at most 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 covalent bonds between a CRISPR polynucleotide and a CRISPR effector protein. There can be about 1 to about 10, about 10 to about 30, about 30 to about 60, or about 60 to about 80 covalent bonds between a CRISPR polynucleotide and a CRISPR effector protein.

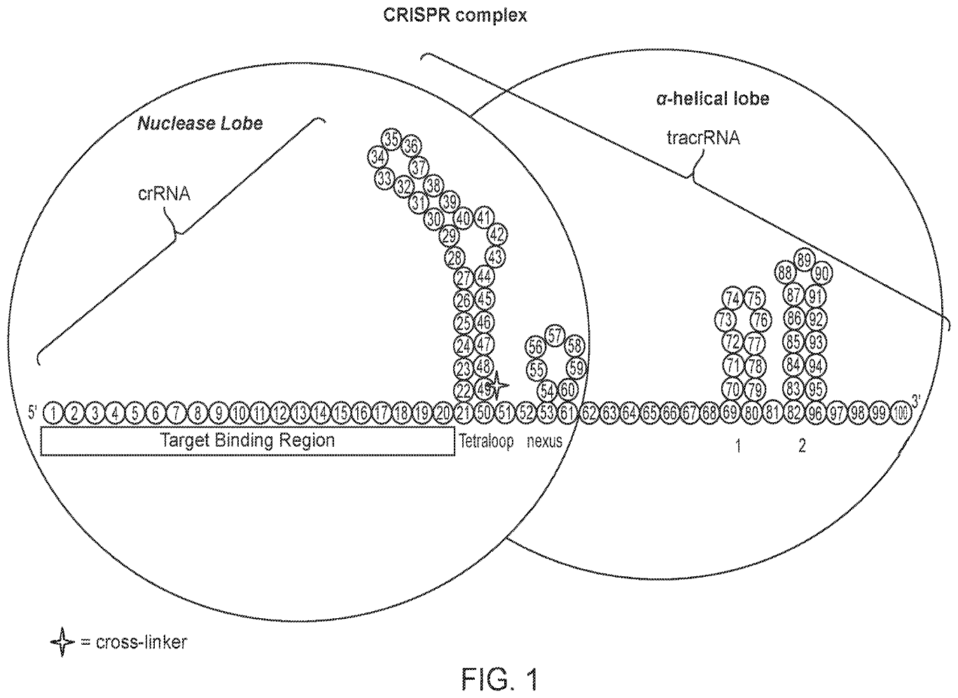

The CRISPR polynucleotide can comprise a backbone that comprises phosphoramide, phosphorothioate, phosphorodithioate, boranophosphate linkage, O-methylphosphoramidite linkages, and/or peptide nucleic acids to control the activity of a CRISPR complex as described herein. Individual nucleotides can be modified so as to add a cross linker to the CRISPR polynucleotide capable of forming a covalent bond with an adjacent amino acid in the CRISPR effector protein. The location of the one or more cross-linkers can be within the tracrRNA region of the CRISPR polynucleotide, e.g., as is diagramed in FIG. 1. The one or more cross linkers can be within a hairpin loop of the sgRNA. The conjugating reactions can be in close proximity to the acceptor molecule in the CRISPR effector protein in order for a covalent bond to form. An embodiment comprises designing the functionalized nucleotide to be within 20, 19, 18, 17, 16, 15, 14, 13, 12, 11, 10, 9, 8, 7, 6, 5, 4, 3, 2, or 1 angstroms of the acceptor molecule of the CRISPR effector protein, e.g., an adjacent amino acid in the CRISPR effector protein, prior to initiation of the cross linking.

The disclosed polynucleotides may comprise a modified nucleotide having a moiety for conjugating the polynucleotides to a protein, which may be referred to as a “crosslinker”). In some cases, the CRISPR polynucleotide (e.g., gRNA, sgRNA, crRNA, or tracrRNA) can comprise at least one crosslinker, at least two crosslinkers, at least five crosslinkers, at least twelve crosslinkers, at least fifteen crosslinkers, at least twenty crosslinkers, at least twenty-five crosslinkers, at least thirty crosslinkers, at least thirty-five crosslinkers, at least forty crosslinkers, at least fifty crosslinkers, at least fifty-five crosslinkers, at least sixty crosslinkers, at least sixty-five crosslinkers, at least seventy crosslinkers, at least seventy-five crosslinkers, or at least 80 crosslinkers. The CRISPR polynucleotide can comprise at most 100, 50, 25, 10, 9, 8, 7, 6, 5, 4, 3, 2, or 1 crosslinker. The CRISPR polynucleotide can comprise about 1 to about 10, about 10 to about 30, about 30 to about 60, or about 60 to about 80 crosslinkers.

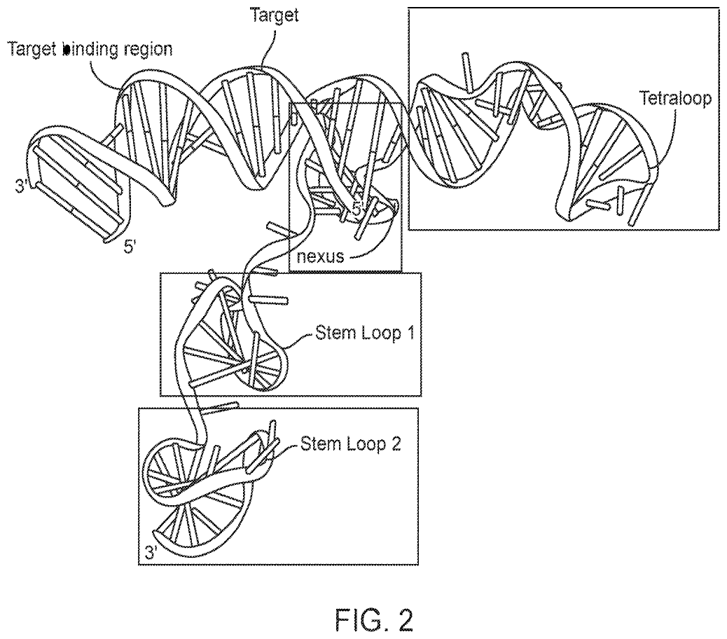

Alternatively, or in combination, the position of the one or more cross linkers can be at any nucleotide of the tracrRNA sequence, or between any two nucleotides of the tracrRNA sequence, outside of the target binding crRNA region. One or more cross-linkers can be present in any stem region: nexus, stem loop 1, stem loop 2, or the tetraloop of the CRISPR polynucleotide (see, e.g., FIG. 2). The one or more cross-linker(s) can be in a hairpin loop or stem of nexus, stem loop 1, stem loop 2, or the tetraloop of the polynucleotide, or any combination thereof. The one or more cross-linker(s) can be present in one hairpin, two hairpins, three hairpins or four hairpins. A loop of a hairpin can comprise one crosslinker, two crosslinkers, three crosslinkers, four crosslinkers, five crosslinkers, six crosslinkers, etc. One, two, three, four, five or more crosslinkers can be in the bulge of the tetraloop.

Alternatively, or in combination with the above, one or more crosslinkers can be at nucleotide position 49 of the sgRNA, wherein nucleotide position 1 is at a 5′ end of the target binding region of the crRNA and nucleotide positions of the sgRNA are numbered consecutively from 5′ to 3′ from nucleotide position 1.

One or more crosslinkers can be at nucleotide position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, or 110 of the sgRNA, wherein nucleotide position 1 is at a 5′ end of the target binding region of the crRNA and nucleotide positions of the sgRNA are numbered consecutively from 5′ to 3′ from nucleotide position 1.

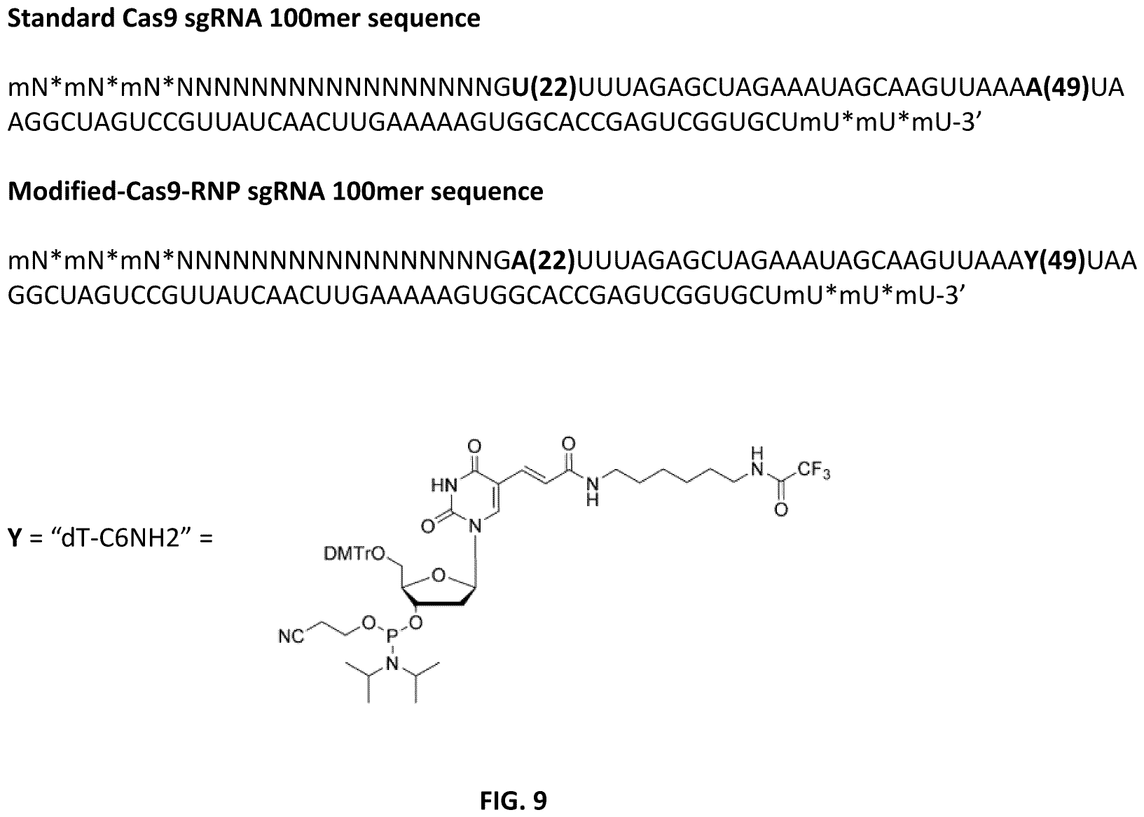

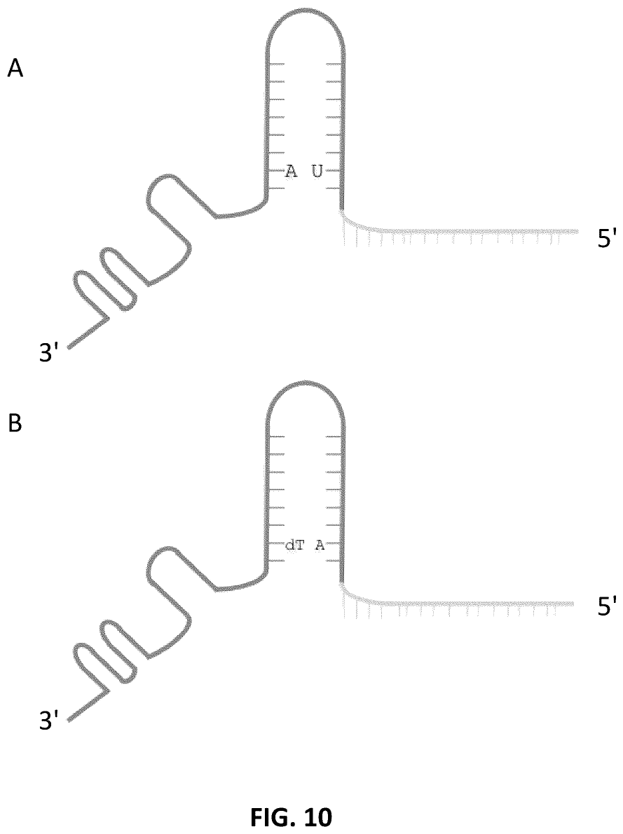

The one or more crosslinkers can be at any uracil or thymidine residue of the sgRNA, where the uracil or thymidine is modified to comprise the conjugating moiety of the crosslinker. The sgRNA can be modified such that hairpin structures of the sgRNA are maintained relative to a structure of a stem loop of an sgRNA lacking the crosslinker. The sgRNA can be modified by a nucleotide swap between complementary pairs of nucleotides within a hairpin structure. An exemplary swap can be a uracil-adenine swap at position 49 of the sgRNA, leaving position 22 with an adenine, as can be seen in FIGS. 9 and 10A modified from the configuration seen in FIG. 9. Alternatively, a deoxyuridine can replace the uridine at position 49 as can be seen in FIG. 10B. Alternatively, a deoxy-adenosine can replace the adenosine at position 22. As a final option, the uridine at position 50 can be substituted with a deoxyuridine.

Alternatively, or in combination with the above, the one or more crosslinkers can be in one hairpin stem, two hairpin stems, three hairpin stems, or four hairpin stems. A hairpin stem can comprise one crosslinker, two crosslinkers, three crosslinkers, four crosslinkers, five crosslinkers, six crosslinkers, seven crosslinkers, eight crosslinkers, etc. The one or more crosslinkers can be in un-base-paired nucleotides between stems. Alternatively, or in combination, one or more crosslinkers can be in one or more nucleotides between stem regions.

The one or more cross-linkers can be located on the backbone of the CRISPR polynucleotide (e.g., gRNA, sgRNA, crRNA, or tracrRNA), or can be included as cross-linker modified nucleotides. Nucleotide modifications can include (a) end modifications, including 5′ end modifications or 3′ end modifications; (b) nucleobase (or “base”) modifications, including replacement or removal of bases; (c) sugar modifications, including modifications at the 2′, 3′ and/or 4′ positions; and (d) backbone modifications, including modification or replacement of the phosphodiester linkages. The CRISPR polynucleotide can comprise a 2′fluoro-arabino nucleic acid, tricycle-DNA (tc-DNA), peptide nucleic acid, cyclohexene nucleic acid (CeNA), ethylene-bridged nucleic acid (ENA), a phosphodiamidate morpholino, (3-(4,4′-Dimethoxytrityl)-1-(2-nitrophenyl)-propan-1-yl-[(2-cyanoethyl)-(N,N-diisopropyl)]-phosphoramidite), or a combination thereof. The CRISPR polynucleotide (e.g., sgRNA) can comprise one or more non-naturally occurring nucleotides or nucleotide analogs, e.g., a nucleotide with phosphorothioate linkage, boranophosphate linkage, or bridged nucleic acids (BNA). The non-naturally occurring nucleotides or nucleotide analogs can be 2′-O-methyl analogs, 2′-deoxy analogs, 2-thiouridine analogs, N6-methyladenosine analogs, or 2′-fluoro analogs.

In some cases, the polynucleotide can comprise modified nucleotides and/or modified internucleotide linkages at the first 1, 2, 3, 4, 5, 6, 7, 8, 9, 10, 11 or 12 nucleotides at the 5′ terminus. In some cases, the polynucleotide can comprise modified nucleotides and/or modified internucleotide linkages at 1, 2, 3, 4, 5, 6, 7, 8, 9, 10, 11 or 12 nucleotides at the 3′ terminus. In some cases, the polynucleotide can comprise modified nucleotides and/or modified internucleotide linkages at the first 1, 2, 3, 4, 5, 6, 7, 8, 9, 10, 11 or 12 nucleotides at the 5′ terminus or 1, 2, 3, 4, 5, 6, 7, 8, 9, 10, 11 or 12 nucleotides at the 3′ terminus. In some cases, the polynucleotide can comprise modified nucleotides and/or modified internucleotide linkages at the first 1, 2, 3, 4, 5, 6, 7, 8, 9, 10, 11 or 12 nucleotides at the 5′ terminus and the first 1, 2, 3, 4, 5, 6, 7, 8, 9, 10, 11 or 12 nucleotides at the 3′ terminus. The modifications can be 2′-O-methyl analogs and/or 3′ phosphorothioate internucleotide linkages.

The CRISPR polynucleotide can comprise one or more modified bases. The one or more modified bases can be 2-aminopurine, 5-bromo-uridine, pseudouridine (Ψ), N\hat{ }mehylpseudouridine (mel P), 5-methoxyuridine (5moU), inosine, or 7-methylguanosine.

In some cases, the 3′ and 5′ termini of a CRISPR polynucleotide can be substantially protected from nucleases e.g., by modifying the 3′ or 5′ linkages (e.g., U.S. Pat. No. 5,849,902 and WO 98/13526). For example, CRISPR polynucleotides can be made resistant by the inclusion of one or more “blocking groups.” The one or more “blocking groups” can be a substituent (e.g., other than OH groups) that can be attached to polynucleotides or nucleomonomers, either as protecting groups or coupling groups for synthesis (e.g., FITC, propyl (CH2-CH2-CH3), glycol (—O—CH2-CH2-O—) phosphate (PO3 2-), hydrogen phosphonate, or phosphoramidite). The one or more blocking groups can be one or more “end blocking groups” or one or more “exonuclease blocking groups” that can protect the 5′ and 3′ termini of the CRISPR polynucleotide, including modified nucleotides and non-nucleotide exonuclease resistant structures.

The one or more end-blocking groups can be a cap structure (e.g., a 7-methylguanosine cap), inverted nucleomonomer, e.g., with 3′-3′ or 5′-5′ end inversions (see, e.g., Ortiagao et al. 1992. Antisense Res. Dev. 2:129), methylphosphonate, phosphoramidite, non-nucleotide groups (e.g., non-nucleotide linkers, amino linkers, conjugates) and the like. The 3′ terminal nucleomonomer can comprise a modified sugar moiety. For example, the 3′-hydroxyl can be esterified to a nucleotide through a 3′→3′ internucleotide linkage. For example, the alkyloxy radical can be methoxy, ethoxy, or isopropoxy. Optionally, the 3′→3′ linked nucleotide at the 3′ terminus can be linked by a substitute linkage. To reduce nuclease degradation, the 5′ most 3′→5′ linkage can be a modified linkage, e.g., a phosphorothioate or a P-alkyloxyphosphotriester linkage.

The CRISPR polynucleotide can comprise one or more labels or tags. The one or more “labels” or “tags” can be a molecule that can be attached to another molecule, e.g., a CRISPR polynucleotide or a segment thereof, to provide a means by which the other molecule can be readily detected. The CRISPR polynucleotide can comprise a label, which can be fluorescent, luminescent, radioactive, enzymatically active, etc. The one or more labels can include fluorochromes, e.g. fluorescein isothiocyanate (FITC), rhodamine, Texas Red, phycoerythrin, allophycocyanin, 6-carboxyfluorescein (6-FAM), 2,7-dimethoxy-4,5-dichloro-6-carboxyfluorescein (JOE), 6-carboxy-X-rhodamine (ROX), 6-carboxy-2,4,7,4,7-hexachlorofluorescein (HEX), 5-carboxyfluorescein (5-FAM) or N,N,N,N-tetramethyl-6-carboxyrhodamine (TAMRA), radioactive labels, e.g. 32P, 35S, 3H; etc. The one or more labels can be a two stage system, where the CRISPR polynucleotide is conjugated to biotin, haptens, etc. having a high affinity binding partner, e.g. avidin, specific antibodies, etc., where the binding partner is conjugated to a detectable label.

The CRISPR polynucleotide (e.g., sgRNA) can comprise one or more stem loops to which one or more stem-loop RNA binding proteins (RBPs) are capable of interacting. These stem loops can be positioned such that the interaction of the CRISPR polynucleotide (e.g., sgRNA) with the CRISPR effector protein (e.g., CRISPR enzyme) or binding of the CRISPR complex with a target DNA is not adversely affected. The one or more stem loops can lie outside the guide sequence of the CRISPR polynucleotide (e.g., the sgRNA). The one or more stem-loop RNA binding proteins can be, e.g., MS2, PP7, Qp, F2, GA, fr, JP501, M12, R17, BZ13, JP34, JP500, KU1, M11, MX1, TW18, VK, SP, Fl, ID2, NL95, TW19, AP205, S1, S1m, 7s, or PRR1.

In some cases, the stem-loop RNA binding protein (RBP) can act as an adaptor protein (i.e., intermediary) that can bind both to the stem-loop RNA and to one or more other proteins or polypeptides, or one or more functional domains. The adaptor protein can recruit effector proteins or fusions that can comprise one or more functional domains. In some cases, the RNA binding protein can be a fusion protein with one or more functional domains.

### 1. Types of Modifications

In some cases, the CRISPR polynucleotide (e.g., gRNA, sgRNA, crRNA, or tracrRNA) can be modified to facilitate conjugating (i.e., “locking”) to a CRISPR effector protein. Modifications for conjugating a CRISPR polynucleotide (e.g., sgRNA) molecule to a CRISPR effector protein can include modifying nucleotides on the sgRNA with functional crosslinking groups.

Modified nucleotides can be introduced into a CRISPR polynucleotide (e.g., sgRNA). Suitable methods are, for example, synthesis methods using (automatic or semi-automatic) oligonucleotide synthesis devices, e.g., in a 3′ to 5′ direction. Such devices may comprise microarrays, polymerase cycling assembly (PCA), microchips, etc.

The CRISPR polynucleotide can comprise a sugar moiety. The sugar moieties can be natural, unmodified sugar, e.g., monosaccharide (e.g., pentose, e.g., ribose, deoxyribose), modified sugars, or sugar analogs. In some cases, the sugar moiety can have or more hydroxyl groups replaced with a halogen, a heteroatom, an aliphatic group, or the one or more hydroxyl groups can be functionalized as an ether, an amine, a thiol, or the like.

The CRISPR polynucleotide can comprise one or more modifications at a 2′ position of a ribose. The one or more modifications at the 2′ position of the ribose can be introduced, e.g., to reduce immunostimulation in a cellular context. The 2′ moiety can be H, OR, R, halo, SH, SR, H₂, HR, R₂ or ON, wherein R is C1-C6 alkyl, alkenyl or alkynyl and halo is F, Cl, Br or I. Examples of sugar modifications include 2′-deoxy-2′-fluoro-oligoribonucleotide (2′-fluoro-2′-deoxycytidine-5′-triphosphate, 2′-fluoro-2′-deoxyuridine-5′-triphosphate), 2′-deoxy-2′-deamine oligoribonucleotide (2′-amino-2′-deoxycytidine-5′-triphosphate, 2′-amino-2′-deoxyuridine-5′-triphosphate), 2′-O-alkyl oligoribonucleotide, 2′-deoxy-2′-C-alkyl oligoribonucleotide (2′-O-methylcytidine-5′-triphosphate, 2′-methyluridine-5′-triphosphate), 2′-C-alkyl oligoribonucleotide, and isomers thereof (2′-aracytidine-5′-triphosphate, 2′-arauridine-5′-triphosphate), azidotriphosphate (2′-azido-2′-deoxycytidine-5′-triphosphate, 2′-azido-2′-deoxyuridine-5′-triphosphate), and combinations thereof. The sugar-modified ribonucleotides can have the 2′ OH group replaced by an H, alkoxy (or OR), R or alkyl, halogen, SH, SR, amino (such as NH₂, NHR, NR₂), or CN group, wherein R is lower alkyl, alkenyl, or alkynyl. The modification at the 2′ position can be a methyl group.

The polynucleotide can comprise one or more nucleobase-modified ribonucleotides. The one or more modified ribonucleotides can contain a non-naturally occurring base (instead of a naturally occurring base), such as uridines or cytidines modified at the 5′-position, e.g., 5′ (2-amino)propyl uridine or 5′-bromo uridine; adenosines and guanosines modified at the 8-position, e.g., 8-bromo guanosine; deaza nucleotides, e.g., 7-deaza-adenosine; and N-alkylated nucleotides, e.g., N6-methyl adenosine.

The nucleobase-modified ribonucleotides can be m5C (5-methylcytidine), m5U (5-methyluridine), m6A (N6-methyladenosine), s2U (2-thiouridine), Um (2′-O-methyluridine), m1A (1-methyl adenosine), m2A (2-methyladenosine), Am (2-1-O-methyladenosine), ms2m6A (2-methylthio-N6-methyladenosine), i6A (N6-isopentenyl adenosine), ms2i6A (2-methylthio-N6isopentenyladenosine), io6A (N6-(cis-hydroxyisopentenyl) adenosine), ms2io6A (2-methylthio-N6-(cis-hydroxyisopentenyl)adenosine), g6A (N6-glycinylcarbamoyladenosine), t6A (N6-threonyl carbamoyladenosine), ms2t6A (2-methylthio-N6-threonyl carbamoyladenosine), m6t6A (N6-methyl-N6-threonylcarbamoyladenosine), hn6A (N6.-hydroxynorvalylcarbamoyl adenosine), ms2hn6A (2-methylthio-N6-hydroxynorvalyl carbamoyladenosine), Ar(p) (2′-0-ribosyladenosine(phosphate)), I (inosine), mi 1 (1-methylinosine), m′lm (1,2′-0-dimethylinosine), m3C (3-methylcytidine), Cm (2T-0-methylcytidine), s2C (2-thiocytidine), ac4C (N4-acetylcytidine), f5C (5-fonnylcytidine), m5Cm (5,2-O-dimethylcytidine), ac4Cm (N4acetyl2TOmethylcytidine), k2C (lysidine), miG (1-methylguanosine), m2G (N2-methylguanosine), m7G (7-methylguanosine), Gm (2′-0-methylguanosine), m22G (N2,N2-dimethylguanosine), m2Gm (N2,2′-0-dimethylguanosine), m22Gm (N2,N2,2′-0-trimethylguanosine), Gr(p) (2′-0-ribosylguanosine(phosphate)), yW (wybutosine), o2yW (peroxywybutosine), OHyW (hydroxywybutosine), OHyW* (undermodified hydroxywybutosine), imG (wyosine), mimG (methylguanosine), Q (queuosine), oQ (epoxyqueuosine), galQ (galtactosyl-queuosine), manQ (mannosyl-queuosine), preQo (7-cyano-7-deazaguanosine), preQi (7-aminomethyl-7-deazaguanosine), G (archaeosine), D (dihydrouridine), m5Um (5,2′-0-dimethyluridine), s4U (4-thiouridine), m5s2U (5-methyl-2-thiouridine), s2Um (2-thio-2′-0-methyluridine), acp3U (3-(3-amino-3-carboxypropyl)uridine), ho5U (5-hydroxyuridine), mo5U (5-methoxyuridine), cmo5U (uridine 5-oxyacetic acid), mcmo5U (uridine 5-oxyacetic acid methyl ester), chm5U (5-(carboxyhydroxymethyl)uridine)), mchm5U (5-(carboxyhydroxymethyl)uridine methyl ester), mcm5U (5-methoxycarbonyl methyluridine), mcm5Um (S-methoxycarbonylmethyl-2-O-methyluridine), mcm5s2U (5-methoxycarbonylmethyl-2-thiouridine), nm5s2U (5-aminomethyl-2-thiouridine), mnm5U (5-methylaminomethyluridine), mnm5s2U (5-methylaminomethyl-2-thiouridine), mnm5se2U (5-methylaminomethyl-2-selenouridine), ncm5U (5-carbamoylmethyl uridine), ncm5Um (5-carbamoylmethyl-2′-0-methyluridine), cmnm5U (5-carboxymethylaminomethyluridine), cnmm5Um (5-carboxymethylaminomethyl-2-L-Omethyluridine), cmnm5s2U (5-carboxymethylaminomethyl-2-thiouridine), m62A (N6,N6-dimethyladenosine), Tm (2′-0-methylinosine), m4C (N4-methylcytidine), m4Cm (N4,2-0-dimethylcytidine), hm5C (5-hydroxymethylcytidine), m3U (3-methyluridine), cm5U (5-carboxymethyluridine), m6Am (N6,T-0-dimethyladenosine), m62Am (N6,N6,0-2-trimethyladenosine), m2′7G (N2,7-dimethylguanosine), m2′2′7G (N2,N2,7-trimethylguanosine), m3Um (3,2T-0-dimethyluridine), m5D (5-methyldihydrouridine), f5Cm (5-formyl-2′-0-methylcytidine), mlGm (1,2′-0-dimethylguanosine), m′Am (1,2-0-dimethyl adenosine)irinomethyluridine), tm5s2U (S-taurinomethyl-2-thiouridine)), imG-14 (4-demethyl guanosine), imG2 (isoguanosine), or ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(Ci-C6)-alkyluracil, 5-methyluracil, 5-(C2-C6)-alkenyluracil, 5-(C2-C6)-alkynyluracil, 5-(hydroxymethyl)uracil, 5-chlorouracil, 5-fluorouracil, 5-bromouracil, 5-hydroxy cytosine, 5-(Ci-C6)-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, and combinations thereof.

The nucleobase-modified ribonucleotide can be Aminopurine, 2,6-Diaminopume (2-Amino-dA), 5-Bromo dU, deoxyuridine, Inverted dT, Inverted Dideoxy-T, dideoxy-C, 5-Methyl dC, Super (T), Super (G), 5-Nitroindole, 2′-O-Methyl RNA Bases, Hydroxymetyl dC, Iso dG, Iso dC, Fluoro C, Fluoro U, Fluoro A, Fluoro G, 2-MethoxyEthoxy MeC, 2-MethoxyEthoxy G, or 2-MethoxyEthoxyT.

a. Chemical Moieties for Conjugation

In some cases, the CRISPR effector protein can be modified to facilitate conjugation to a CRISPR polynucleotide (e.g., sgRNA). The CRISPR polynucleotide (e.g., sgRNA) can comprise one or more moieties for conjugating the CRISPR polynucleotide to a CRISPR effector protein, which otherwise may be called “crosslinkers.” The one or more cross linkers can be a functional group that forms a covalent bond, e.g., between polymers, such as isocyanates. The one or more cross-linkers can be formaldehyde or glutaraldehyde. The conjugating can involve bio conjugation. Bio conjugation crosslinking reagents can contain reactive groups that react with functional groups such as amines and sulfhydryls. Bio conjugation cross linkers can include sulfhydryl reactive groups such as maleimides, haloacetyls, aziridines, acryloyls, alkoxyamine, arylating agents, vinylsulfones, pyridyl disulfides, TNB-thiols, dithiol phosphoramidite DTPA etc. Bio conjugation cross linkers can also include amine reactive crosslinker reactive groups such as succinimidyl esters (NHS esters), sulfonyl chloride, aldehyde, carbodiimide, acyl azide, aryl azide, anhydride, fluorobenzene, carbonate, imidoester, epoxide, fluorophenyl ester, phosphoramidite, etc.

Further non-limiting examples of cross-linkers can be derived from the following compounds: thiol+thiol, thiol+maleimide, NHS ester+amine, carboxylic acid+NHS+amine, azide+phosphine (Staudinger ligation), carbonyl compound+amine, carbonyl compound+O-substituted hydroxylamines, diazirine+C-H/O—H, N—H, haloacetate+thiol, azide+alkyne, nitrone+alkyne, nitrile oxide+alkyne, tetrazine+alkene, 4-thiouridine, 5′-azideuridine, 5-bromouridine, 8-azidoadenosine, 5-((4-Azidophenacyl)thio)uridine.

The CRISPR polynucleotide (e.g., sgRNA) can be modified to comprise one or more unnatural nucleotides. An unnatural nucleotide can comprise a nucleotide which contains one or more modifications to the base, sugar, and/or phosphate moiety. The one or more modifications can comprise one or more chemical modifications. The one or more modifications can be, for example, of a 3′OH or 5′OH group, of the backbone, of the sugar component, and/or of the nucleotide base (e.g., purine or pyrimidine). The one or more modifications can include addition of one or more linker molecules for crosslinking. The one or more linker molecules can be configured to form covalent bonds with an amino acid. The one or more linker molecules can be configured to form non-covalent bonds with an amino acid. In one aspect, a modified base comprises a base other than adenine, guanine, cytosine, or thymine (in modified DNA), or a base other than adenine, guanine, cytosine or uracil (in modified RNA). In some embodiments, a modification is to a modified form of adenine, guanine, cytosine or thymine (in modified DNA) or a modified form of adenine, guanine, cytoside or uracil (in modified RNA). An unnatural nucleotide can be a nucleotide covalently modified at sugar, internucleotide phosphodiester bonds, purine or pyrimidine residues to comprise a functional group of a covalent linker. see, e.g., Sletten et al., Angew. Chem. Int. Ed. (2009) 48:6974-6998; Manoharan, M. Curr. Opin. Chem. Biol. (2004) 8: 570-9; Behlke et al., Polynucleotides (2008) 18: 305-19; Watts, et al., Drug. Discov. Today (2008) 13: 842-55; Shukla, et al., ChemMedChem (2010) 5: 328-49. An unnatural nucleotide can include a nucleotide modified, for example covalently modified, at a sugar, internucleotide phosphodiester bond, purine or pyrimidine residues to comprise a functional group. The covalent linker can be a chemical moiety selected from the group consisting of carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs. The chemical bonds can be based on carbamates, ethers, esters, amides, imines, amidines, aminotrizines, hydrozone, disulfides, thioethers, thioesters, phosphorothioates, phosphorodithioates, sulfonamides, sulfonates, sulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

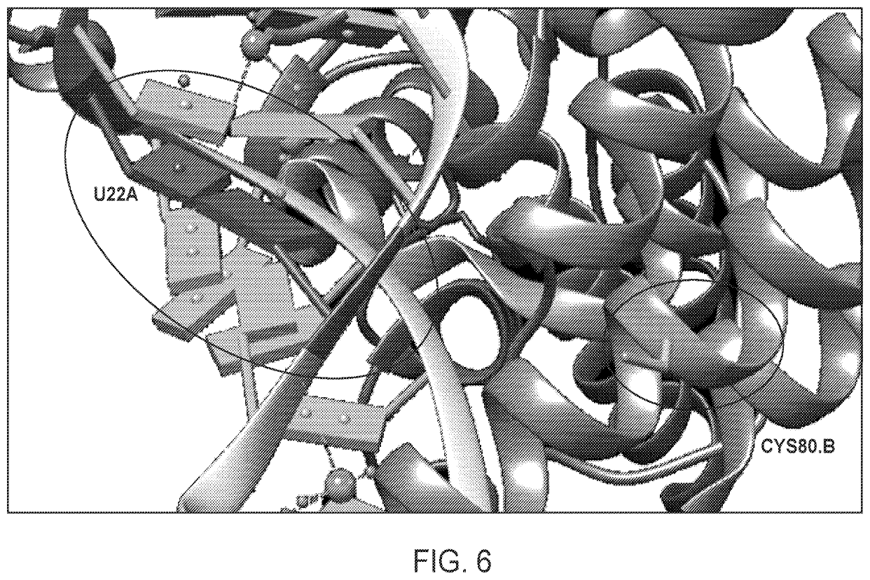

For example, unnatural nucleotides of the CRISPR polynucleotide (e.g., sgRNA) can comprise maleimides to crosslink to nearby cysteine amino acids in the CRISPR effector protein, forming a thioether bond. One technique for integrating unnatural nucleotides capable of crosslinking the CRISPR effector protein to the CRISPR polynucleotide (e.g., sgRNA) can involve modifying nucleotides of the CRISPR polynucleotide (e.g. sgRNA) with chemical groups that react with cysteines found in the CRISPR effector protein, such as maleimides. The unnatural nucleotides comprising maleimides can crosslink the CRISPR polynucleotide to the CRISPR effector protein by reacting with a thiol side chain of a Cysteine of the CRISPR effector protein. FIG. 6 shows a crystal structure of an exemplary location of an unnatural nucleotide to crosslink to an adjacent cysteine of a CRISPR effector protein comprising a Cas9 nuclease. Specifically, the structure shows an unnatural nucleotide, nucleotide position 22, (white circle on left) and an adjacent amino acid on the CRISPR effector protein, cysteine at position 80 of the CRISPR effector protein (white circle on right). As described above, sgRNA positions 22 and 49 can be modified with a uridine to adenine switch, leaving RNA nucleotide position 49 as a uracil (U49). An unnatural nucleotide at U49 can be integrated by modification of the sugar molecule of U49 to include crosslinking moieties that interact with Cys80 on SpCas9. Other positions of a sgRNA with a wild type tracr RNA sequence configured to complex with a Cas9 nuclease which could be modified with a uridine to adenine switch are U72/A77, U71/A78, and U94/A84. Uracil nucleotides at positions 77, 78 and 84 of the sgRNA can be modified with crosslinking groups described herein so as to form covalent bonds with adjacent amino acids of the Cas9 nuclease.

In one exemplary method of modifying a nucleotide with a linker group for forming a covalent bond, a phosphoramidite nucleotide is reacted with a nucleophile attached to a spacer and a maleimide group. The incoming nucleophile replaces the linker group of the phosphoramidite nucleotide, leaving a spacer attached to the maleimide. When in proximity to a cysteine with a thiol group, such as when complexed with a CRISPR effector protein, the maleimide can form a covalent thioether bond with the cysteine under physiological conditions.

One technique for crosslinking the CRISPR effector protein to the CRISPR polynucleotide (e.g., sgRNA) can involve modifying nucleotides of the CRISPR polynucleotide (e.g., sgRNA) to create unnatural nucleotides with chemical groups that react with primary amines found on the side chain of lysine in the CRISPR effector protein, such as NHS esters, epoxide, aldehyde, acyl azide, etc.

### B. Photo Reactive Cross-Linkers for Conjugation

The cross-linkers of the CRISPR polynucleotides may be activatable. The CRISPR polynucleotides provided herein can comprise one or more photo labile linkers, e.g., aryl azides (phenyl azides) and diazirines. The one or more photo labile groups (linkers) can be used in photo-chemical crosslinking reactions that can use energy from light to be initiated. The one or more photo labile groups can be chemically inert compounds that become reactive when exposed to ultraviolet or visible light. The one or more photo labile groups incorporated into crosslinking compounds for use in bio conjugation techniques can be aryl azides, azido-methyl-coumarins, benzo-phenones, anthraquinones, diazo compounds, diazirines, and psoralen derivatives.

The CRISPR polynucleotides (e.g., sgRNA) can be modified with psoralen for crosslinking reactions with the CRISPR effector protein. Psoralen can react exclusively with RNA or DNA and can be used to label nucleic acids or to crosslink the CRISPR effector protein with the CRISPR polynucleotide. Nucleotides modified with photo labile groups that can be incorporated in to the CRISPR polynucleotide can include 4-thio-UTP, 5-azido-UPT, 5-bromo-UTP, 8 azido-ATP, -APAS-UTP, 8-N(3)AMP, 5-[N-(4-benzoyl-benzoyl)-3-aminoallyl]-deoxyuridine triphosphate (BP-dUTP, benzophenone modified), 5-[N-(4-azido-2,3,5,6-tetreafluorobenzoyl)-3-aminoallyl]-deoxy-uridine triphosphate (FAB-dUTP, perfluorinated aryl azide modified), 5-{N-[4-[3-(trifluoromethyl)-diazirin-3-yl]benzoyl]-3-aminoallyl}-deoxyuridine triphosphate (DB-dUTP, diazirine modified), and 5-[N-(p-azidobenzoyl)-3-aminoallyl]-deoxyuridine triphosphate (AB-dUTP, aryl azide modified).

Crosslinking can be by photoinitiation. A light emitting device, producing wavelengths in and near the ultraviolet range, can be placed such that a solution carrying the CRISPR polynucleotide (e.g., sgRNA) bound to the CRISPR effector protein, e.g., by hydrogen bonding, can be exposed to the wavelength upon passing by the light emitting device. This exposure can lead to the photoinitiation of the photo labile group and the formation of a covalent bond linking the CRISPR polynucleotide (e.g., sgRNA) to the CRISPR effector protein as described above.

The wavelength of the light for photoinitiation can range from 220-465 nm. The intensity of light in the exposure protocol can be about 15, 20, 25, 35, 40, 50, 70, 90, 110, 120, 140, 160, 175, 190, 200, 220, 240, 260 280, 300, 320, 340, 360, 380, 400, 420, 440, 460, 480, 500, 520, 540, 560, 580, 600, 620, 650, 675, 700, 720, 745, 765, 790, 810, 830, 850, 870, 900, 920, 945, 965, 985, 1000, 1025, 1050, 1080, 1100, 1125, 1150, 1175, 1200, 1240, 1275, 1290, 1320, 1350, 1380, 1400, 1420, 1450, 1470, 1490, 1520, 1540, 1560, 1600, 1630, 1650, 1670, 1700, 1720 or 1750 mW/cm². The power wattage of the light used in the exposure protocol can be about 50, 70, 80, 90, 100, 120, 140, 160, 175, 190, 210, 230, 250, 270, 290, 310, 330, 250, 370, 390, 420, 450, 480, 500, 530, 550, 570, 600, 620, 650, 670, 700, 720, 750, 770, 800, 820, 850, 870, 900, 920, 950, 970, 1000, 1020, 1050, 1070, 1100, 1120, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400 4500, 4600, 4700, 4800, 4900, 5000, 5100, 5200, 5300, 5400, 5500, 5600, 5700, 5800, 5900, or 6000 W, as measured by an OAI 306 UV power meter.

The duration of exposure can be from 1 second to 30 minutes. The exposure protocol can comprise continuous exposure or pulsed exposure or both. The pulse exposure can be uniform or of varying durations. Exposure time to initiate crosslinking can be dependent upon the crosslinker chosen. For instance, upon exposure to UV light, diazirines can produce a reactive carbene with a half-life on the scale of nanoseconds. Aryl azides can form a reactive carbene upon exposure to UV light with a half-life on the scale of milliseconds. Exposure time can also be dependent on the distance from the available C—H group for reaction to the carbene.

The one or more photo labile groups used in crosslinking can be activated by a wavelength of light. The wavelength can provide excitation of the electron shell of the photo labile groups through a photon at a particular frequency. The reaction can occur upon excitation, which can lend flexibility to the crosslinking reaction with regard to the timing of the covalent bond formation. The one or more photo labile groups can be chosen to be activated by ultraviolet wavelengths.

Cross-linking can occur in vitro. Physiological conditions can be used to ensure the proper folding and attachment of the CRISPR effector protein to the CRISPR polynucleotide. Physiological conditions can include solutions with reagents, e.g., 20 mM Tris, pH 7.5, 100 mM KCl, 5 mM MgCl₂, 1 mM DTT and 5% (v/v) glycerol. Temperatures can be about 25° C. or 37° C.

To facilitate the formation of a CRISPR complex from individual CRISPR polynucleotides (e.g., sgRNA) and CRISPR effector proteins, a ratio (e.g., molar ratio) (CRISPR polynucleotide: CRISPR effector protein) provided can be about 0.001:1, 0.01:1, 0.1:1, 0.2:1, 0.3:1, 0.4:1, 0.5:1, 0.6:1, 0.7:1, 0.8:1, 0.9:1, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 25:1, 30:1, 40:1, 50:1, 60:1, 70:1, 80:1, 90:1, 100:1, and any variation in between. The ratio (e.g., molar ratio) (CRISPR polynucleotide: CRISPR effector protein) can be about 0.001:1 to about 0.01:1, about 0.01 to about 0.1:1, about 0.1:1 to about 1:1, about 1:1 to about 10:1, or about 10:1 to about 100:1, or about 100:1 to about 1000:1.

Cross linking can also occur in vivo, e.g., after a cell is contacted with a solution of CRISPR effector protein, unbound or bound to the CRISPR polynucleotide. In some cases, the CRISPR polynucleotide (e.g., sgRNA) and/or CRISPR effector protein (e.g., Cas9) can be expressed from a nucleic acid in the cell. The cell can be exposed to UV light in order to lock (e.g., covalently cross-link) the CRISPR polynucleotide (e.g., sgRNA) to the CRISPR effector protein (e.g., Cas9). The cell can be ectodermal (e.g., neurons and fibroblasts), mesodermal (e.g., cardiac muscle cells), endodermal (e.g., pancreatic cells), epithelial (e.g., lung and nasal passageways), neutrophils, eosinophils, basophils, lymphocytes, osteoclasts, endothelial cells, hematopoietic, red blood cells, etc. The cell can be derived from specific cell lines such as CHO cells (e.g., CHOK1); HEK293 cells; Caco2 cells; U2-OS cells; NIH 3T3 cells; NSO cells; SP2 cells; DG44 cells; K-562 cells, U-937 cells; MC5 cells; IMR90 cells; Jurkat cells; HepG2 cells; HeLa cells; HT-1080 cells; HCT-116 cells; Hu-h7 cells; Huvec cells; and Molt 4 cells. Examples of other cells applicable to the scope of the present disclosure can include stem cells, embryonic stem cells (ESCs) and induced pluripotent stem cells (iPSCs)., MSC-1, K562, etc.

Timing of the cross linking can be dependent upon the crosslinking functional group chosen. In the case of a photo reactive cross linker, the exposure of the CRISPR effector protein/CRISPR polynucleotide complex to, e.g., light, can occur after the CRISPR effector protein and CRISPR polynucleotide are mixed in solution together.

The duration of exposure to the light, e.g., UV light, can be from 1 second to 30 minutes. The duration of exposure to the light, e.g., UV light, can be less than two seconds, less than five seconds, less than ten seconds, less than twenty seconds, less than 30 seconds, less than 45 seconds, less than 50 seconds, less than one minute, less than two minutes, less than five minutes, less than ten minutes, less than fifteen minutes, less than twenty minutes, less than 30 minutes, less than 45 minutes.

### 2. Modifications to Modulate CRISPR Activity

In some cases, e.g., to increase the effectiveness of a CRISPR polynucleotide, e.g., gRNA or sgRNA, one or more modifications can be added to the CRISPR polynucleotide, e.g., gRNA or sgRNA that lower the off-target editing activity of the CRISPR polynucleotide in complex with a CRISPR enzyme. The one or more modifications can be at various locations, including at a sugar moiety, a phosphodiester linkage, and/or a base. For example, the CRISPR polynucleotide can comprise a backbone that comprises phosphoramide, phosphorothioate, phosphorodithioate, boranophosphate linkage, O-methylphosphoramidite linkages, and/or peptide nucleic acids. The one or more can comprise a 2′fluoro-arabino nucleic acid, tricycle-DNA (tc-DNA), peptide nucleic acid, cyclohexene nucleic acid (CeNA), locked nucleic acid (LNA), a locked nucleic acid (LNA) nucleotide comprising a methylene bridge between the 2′ and 4′ carbons of the ribose ring, bridged nucleic acids (BNA), ethylene-bridged nucleic acid (ENA), a phosphodiamidate morpholino, or a combination thereof.

The CRISPR polynucleotide modified with at least one unnatural nucleotide to crosslink to a CRISPR effector protein may comprise a sequence configured to facilitate a cleavage property. The cleavage property of the CRISPR polynucleotide modified with at least one unnatural nucleotide to crosslink to a CRISPR effector protein can be altered by a cleavable element that can alter the propensity of cleavage of the CRISPR polynucleotide at the point of its incorporation, under appropriate conditions. A “cleavable element” can comprise natural nucleotides or one or more modified nucleotides. The cleavable element can be incorporated into the CRISPR polynucleotide (e.g., sgRNA) during nucleic acid synthesis.

Two or more cleavable elements in a CRISPR polynucleotide can have different cleavage characteristics, e.g., the two or more cleavable elements, when incorporated into a CRISPR polynucleotide (e.g., sgRNA), can be selectively cleaved in each other's presence by using different agents and/or reaction conditions.

As used herein, the terms “cleaving,” “cleaved” and “cleavage” can all relate to the scission of the CRISPR polynucleotide (e.g., sgRNA) substantially at each point of occurrence of a cleavable element in the CRISPR polynucleotide (e.g., sgRNA).

The cleavage can be initiated by an agent. The agent can be, e.g., a chemical entity or physical force that causes the cleavage of a cleavable element. The agent can be a chemical or combination of chemicals, a biomolecule or combination of biomolecules, normal or coherent (laser) visible or ultraviolet (UV) light, heat or other forms of electromagnetic energy. In some cases, a combination of agents, e.g., two or more agents, can be used simultaneously or sequentially to cleave a CRISPR polynucleotide (e.g., sgRNA). By simultaneously is meant a CRISPR polynucleotide (e.g., sgRNA) can be exposed to the two or more agents at the same time, although the two or more agents can react with the CRISPR polynucleotide (e.g., sgRNA) one at a time. By sequentially it is meant that the CRISPR polynucleotide (e.g., sgRNA) can be contacted with one agent and then a second agent at a later time.

A CRISPR polynucleotide comprising one or more unnatural nucleotides to crosslink to a CRISPR effector protein can comprise more than one type of cleavable element. In some examples, the first cleavable element and the second cleavable element have the same cleavage characteristics. In some examples, the second cleavable element has different cleavage characteristics than the first cleavable element. For example, the first cleavable element can be a photocleavable linker and the second cleavable element can be susceptible to cleavage by a chemical nuclease. In another example, the first cleavable element can be susceptible to cleavage by a chemical nuclease, and the second cleavable element can be engineered to be photocleavable allowing orthogonal treatment regimens to be applied. In some cases, the same cleavable element can have more than one type of cleavage characteristic. The first and second cleavable element can be any cleavable element described herein.

A cleavable element (e.g., cleavable linker) can refer to an entity that can connect two or more constituents of a CRISPR polynucleotide (e.g., sgRNA or crRNA) that renders the CRISPR polynucleotide (e.g., sgRNA or crRNA) susceptible to cleavage under appropriate conditions. For instance, the appropriate conditions can be exposure to UV light. The cleavable linker can comprise one or more modified or unmodified nucleotides, which are susceptible to scission under the appropriate conditions.

The cleavable linker can comprise a modified internucleoside linkage. The modified internucleoside linkage can be an internucleotide linkage that has a phosphorus atom or those that do not have a phosphorus atom. Internucleoside linkages containing a phosphorus atom therein include, for example, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, P-ethyoxyphosphodiester, P-ethoxyphosphodiester, P-alkyloxyphosphotriester, methylphosphonate, thionophosphoramidates, thionoalkylphosphonates, thionoalkylphosphotriesters, selenophosphates and boranophosphates, and nonphosphorus containing linkages, e.g., acetals and amides, such as are known in the art, having normal 3′-5′ linkages, 2′-5′ linked analogs of these, and those having inverted polarity wherein one or more internucleotide linkages is a 3′ to 3′, 5′ to 5′ or 2′ to 2′ linkage. Polynucleotides having inverted polarity can comprise a single 3′ to 3′ linkage at the 3′-most internucleotide linkage i.e. a single inverted nucleoside residue which may be abasic (the nucleobase is missing or has a hydroxyl group in place thereof).

Non-phosphorus containing internucleoside linkages include short chain alkyl, cycloalkyl, mixed heteroatom alkyl, mixed heteroatom cycloalkyl, one or more short chain heteroatomic and one or more short chain heterocyclic. These internucleoside linkages include but are not limited to siloxane, sulfide, sulfoxide, sulfone, acetyl, formacetyl, thioformacetyl, methylene formacetyl, thioformacetyl, alkeneyl, sulfamate; methyleneimino, methylenehydrazino, sulfonate, sulfonamide, amide and others having mixed N, O, S and CH2 component parts. Other modified internucleoside linkages that do not contain a phosphorus atom therein include, —CH2-NH—O—CH2-, —CH2-N(CH3)-O—CH2- (known as a methylene (methylimino)backbone), —CH2-O—N(CH3)-CH2-, —CH2-N(CH3)-N(CH3)-CH2- and —O—N(CH3)-CH2-CH2-.

The cleavable linker can be non-nucleotide in nature. A “non-nucleotide” can refer to any group or compound that can be incorporated into a polynucleotide chain in the place of one or more nucleotide units, including either sugar and/or phosphate substitutions. The group or compound can be abasic in that it does not contain a commonly recognized nucleotide base, such as adenosine, guanine, cytosine, uracil or thymine, for example at the C1 position of the sugar.

Non-nucleotidic linkers can be e.g. abasic residues (dSpacer), oligoethyleneglycol, such as triethyleneglycol (spacer 9) or hexaethylenegylcol (spacer 18), or alkane-diol, such as butanediol. The spacer units can be preferably linked by phosphodiester or phosphorothioate bonds. The linker units may appear just once in the molecule or may be incorporated several times, e.g. via phosphodiester, phosphorothioate, methylphosphonate, or amide linkages. Further preferred linkers are alkylamino linkers, such as C3, C6, C12 aminolinkers, and also alkylthiol linkers, such as C3 or C6 thiol linkers. In some examples, heterobifunctional and homobifunctional linking moieties may be used to conjugate peptides and proteins to nucleotides. Examples include 5′-Amino-Modifier C6 and 3′-Amino-Modifier C6 reagents.

a. CRISPR ON

Provided herein are CRISPR ON polynucleotides that can be covalently crosslinked to CRISPR effector proteins to form CRISPR ON complexes. A CRISPR ON polynucleotide can comprise (i) a guide sequence configured to anneal to a target sequence in a target molecule (ii) a sequence (e.g., a tracrRNA sequence) configured to bind to a CRISPR effector protein, and (iii) a first sequence element 5′ of the guide sequence. The first sequence element 5′ of the guide sequence can be referred to as a polynucleotide leader sequence. The first sequence element can comprise a secondary structure, e.g., a stem loop. The stem loop can comprise from about 3 base pairs (bp) to about 30 bp. The 5′ end of the first sequence element can be annealed to the base in the sequence element immediately 5′ to the guide sequence. In some cases, the 5′ end of the first sequence element is annealed to the guide sequence. The CRISPR ON polynucleotide can further comprise a first cleavable element, e.g., a first non-naturally occurring cleavable element, e.g., a photolabile linker. The cleavable element can be positioned immediately 5′ of the guide sequence. The cleavable element can be susceptible to cleavage by light, small molecule, or one or more cellular processes. The polynucleotide leader sequence can interfere with the ability of the guide sequence to anneal to a target sequence.

Complexes comprising a CRISPR effector protein and the crosslinked CRISPR ON polynucleotide can be assembled. A CRISPR complex comprising a crosslinked CRISPR ON polynucleotide with a first sequence element 5′ of the guide sequence and a CRISPR effector protein can have a lower target specific activity than a CRISPR complex comprising a crosslinked CRISPR polynucleotide without the first sequence element; for example, the activity can be about 2-fold to about 100 fold lower. Provided herein are methods for the tunable targeting of a CRISPR complex to a target nucleic acid, e.g., DNA. The methods can comprise cleaving the cleavable element with a cleavage agent, thereby releasing the first sequence element 5′ of the guide sequence. For example, the cleavable element can be a photolabile linker, and the photolabile linker can be cleaved when exposed to light. Cleaving the cleavable linker can result in a CRISPR complex with higher target-specific cleavage activity than the CRISPR complex before the cleavage.

A CRISPR ON polynucleotide or CRISPR ON/OFF polynucleotide can comprise a first sequence element 5′ of the guide sequence. The first sequence element 5′ of the guide sequence can be referred to as a polynucleotide leader sequence. A CRISPR complex comprising a CRISPR polynucleotide with a polynucleotide leader sequence and a CRISPR effector protein crosslinked to the CRISPR polynucleotide can have a lower activity than a CRISPR complex comprising a CRISPR polynucleotide without the polynucleotide leader sequence. Removal of the polynucleotide leader sequence can result in a CRISPR complex with an increased activity (CRISPR ON).

i. Length of the Polynucleotide Leader Sequence

The polynucleotide leader sequence can range from about 1 nucleotide to about 50 nucleotides, e.g., about 5 nucleotides to about 30 nucleotides, about 10 nucleotides to about 20 nucleotides, about 15 nucleotides, or at least 4 nucleotides, 3 nucleotides to about 15 nucleotides, e.g., about 5 nucleotides to about 15 nucleotides, about 3 nucleotides to about 10 nucleotides, about 3 to about 15 nucleotides, or about 3 nucleotides to about 12 nucleotides, about 4 nucleotides to about 13 nucleotides, about 3 nucleotides to about 18 nucleotides, about 4 nucleotides to about 19 nucleotides, from 4 nucleotides to about 30 nucleotides, from 4 nucleotides to about 25 nucleotides, from 5 nucleotides to about 12 nucleotides, from 5 nucleotides to about at least 4 nucleotides, or 30 or fewer nucleotides in length.

ii. Composition of the Polynucleotide Leader Sequence

The polynucleotide leader sequence can comprise ribonucleotides and/or deoxyribonucleotides. The polynucleotide leader sequence can comprise non-canonical nucleotides or nucleotide analogues. The polynucleotide leader sequence can comprise any nucleotide or modified nucleotide or internucleotide linkage described herein. In some cases, the polynucleotide leader sequence can comprise any linker described herein.

iii. Secondary Structure in the Polynucleotide Leader Sequence

The polynucleotide leader sequence can form, or be designed to form, secondary structure. The secondary structure can be, e.g., a stem loop structure. The stem of the stem loop can comprise at least about 3 bp comprising complementary X and Y sequences (where X represents the sequence of one strand of the stem and Y represents the sequence of the other strand of the stem). The stem can comprise at least (or at most) 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 base pairs. The stem can comprise a double stranded domain ranging from 1-20 bp, or from 2-5 bp, 2-9 bp, 3-10 bp, 4-9 bp, 5-10 bp, 5-20 bp, 6-20 bp, 7-20 bp, 8-20 bp etc. In some cases, the two strands of the stem can be covalently cross-linked.

The stem loop can comprise a single-stranded loop. The single-stranded loop can range from 1-50 bases, e.g., 3-5 bases, 3-7 bases, 4-10 bases, 5-20 bases, 6-25 bases, 3-25 bases, 3-30 bases, 4-30 bases, or 4-50 bases.

The 5′ most base of the stem loop, or of the polynucleotide leader sequence, can anneal to a base in the polynucleotide leader sequence immediately 5′ of the guide sequence. In some cases, the 5′ most base of the polynucleotide leader sequence can anneal to a base 1-20 bases 3′ of the 5′ most base of the guide sequence, e.g., 2 bases, 3 bases, 4 bases, 5 bases, 6 bases, 7 bases, 8 bases, 9 bases, 10 bases, 11 bases, 12 bases, 15 bases, or 20 bases 3′ of the 5′ most base of the guide sequence. In some cases, the polynucleotide leader sequence does not comprise a base that base pairs to a base in the guide sequence.

The polynucleotide leader sequence can form a hairpin loop or stem-loop structure comprising one or more bulges (regions of single stranded sequence; these regions can correspond to positions comprising less than 100% sequence base-pairing in the secondary structure). The number, length, and/or position of the one or more bulges can vary and can affect the overall stability of the stem-loop structure. The polynucleotide leader sequence can comprise 2, 3, 4, 5 or more bulges when optimally folded.

In some cases, the polynucleotide leader sequence can comprise non-polynucleotide moieties. The non-nucleotide moieties in the polynucleotide leader sequence can be biotin, antibodies, peptides, affinity, reporter or protein moieties (such as NHS esters or isothiocyanates), digoxigenin, enzymes such as alkaline phosphatase etc.

In some cases, the polynucleotide leader sequence lacks secondary structure. The polynucleotide leader sequence can comprise or consist of a single stranded contiguous stretch of nucleotides.

The melting temperature of a stem loop formed by the polynucleotide leader sequence can be about 25° C. to about 60° C., or about 30° C. to about 50° C., or about 40° C. to about 50° C.

iv. Activity Reduction by Polynucleotide Leader Sequence

A CRISPR complex comprising a CRISPR polynucleotide with a polynucleotide leader sequence and a CRISPR effector protein crosslinked to the CRISPR polynucleotide can have a lower activity than a CRISPR complex comprising a CRISPR polynucleotide without the polynucleotide leader sequence. In some cases, the activity is at least (or at most) 0.1-fold, 0.25 fold, 0.5 fold, 0.75 fold, 1 fold, 2 fold, 5 fold, 10 fold, 50 fold, 100 fold, or 1000 fold lower. In some cases, a CRISPR complex comprising a CRISPR polynucleotide with a polynucleotide leader sequence and a CRISPR effector protein has no activity. The activity can be, e.g., enzymatic activity or transcriptional activation activity. For example, when the CRISPR effector protein is a catalytically active Cas protein, the CRISPR complex can be unable to cleave target nucleic acid. In another example, when the CRISPR effector protein is a catalytically dead Cas protein fused to a transcription activation domain, the CRISPR complex can be unable to activate transcription of a target gene.

v. Removing the Polynucleotide Leader Sequence

The CRISPR polynucleotide can comprise one or more cleavable elements to permit release of the polynucleotide leader sequence. The one or more cleavable elements can be between the polynucleotide leader sequence and the guide sequence. In some cases, the one or more cleavable elements are within the polynucleotide leader sequence. In some cases, at least one cleavable element is within the polynucleotide leader sequence and at least one cleavable element is between the polynucleotide leader sequence and the guide sequence. In some cases, the one or more cleavable elements are positioned 5′ of the guide sequence. The one or more cleavable elements can be at least, or at most, 2, 3, 4, 5, 6, 7, 8, 9 or 10 cleavable elements. In some cases, the one or more cleavable elements are positioned such that following cleavage, part of the polynucleotide leader sequence (e.g., 1 base, 2 bases, 5 bases, or 10 bases) remains covalently linked to the guide sequence. In some cases, the one or more cleavable elements are positioned such that following cleavage, none of the polynucleotide leader sequence remains covalently attached to the guide sequence.

The one or more cleavable elements can be any cleavable element described herein. The one or more cleavable elements can be the same type of cleavable element or different types of cleavable elements.

The CRISPR polynucleotide can be cleaved at the one or more cleavable elements while the CRISPR polynucleotide is not bound to a CRISPR effector protein. The CRISPR polynucleotide can be cleaved at the one or more cleavable elements while the CRISPR polynucleotide is crosslinked with a CRISPR effector protein. The CRISPR polynucleotide can be cleaved at the one or more cleavable elements while the CRISPR polynucleotide is crosslinked with a CRISPR effector protein and bound to a target sequence. In some cases, the polynucleotide leader sequence prevents the CRISPR polynucleotide from crosslinking with a CRISPR effector protein or reduces the ability of the CRISPR polynucleotide to crosslink to the CRISPR effector protein relative to a CRISPR polynucleotide that lacks the polynucleotide leader sequence; cleavage of the polynucleotide leader sequence from the CRISPR polynucleotide can increase the ability of the CRISPR polynucleotide to bind a CRISPR effector protein.

The CRISPR polynucleotide can be cleaved at the one or more cleavable elements in vitro. The CRISPR polynucleotide can be cleaved at the one or more cleavable elements while in a cell or organism, e.g., mouse, rabbit, goat, primate, e.g., chimpanzee, gorilla, or human. The timing of the cleaving of the CRISPR polynucleotide at the one or more cleavable elements can vary. For example, the one or more cleavable elements can be cleaved immediately after the CRISPR polynucleotide is introduced into a cell or organism, or at least (or at most) 0.25, 0.5, 0.75, 1, 2, 3, 4, 5, 6, 7, 8, 9, 10, 11, 12, 13, 14, 15, 16, 17, 18, 19, 20, 21, 22, 23, 24, 48, 72, or 96 hours after introduction into a cell or organism.

A CRISPR polynucleotide can be exposed to a cleavage agent once. The CRISPR polynucleotide can be subjected to a cleavage agent more than once, e.g., 2 times, 3 times, 5 times, or 10 times. The CRISPR polynucleotide can be exposed to more than one type of cleavage agent, e.g., at least (or at most) 2, 3, 4, 5, 6, 7, 8, 9, or 10 cleavage agents. A CRISPR polynucleotide can be exposed to a cleavage agent for varying durations. For example, a CRISPR polynucleotide can be exposed to a cleavage agent for 0.1 min, 0.5 min, 1 min, 2 min, 3 min, 4 min, 5 min, 10 min, 30 min, 60 min, 2 hr, 4 hr, 6 hr, 12 hr, 24 hr, 48 hr, 72 hr, or 96 hr.

In some cases, a sample comprises a plurality of CRISPR polynucleotides, and a cleavage agent can be used to cleave a certain percentage of the CRISPR polynucleotides. For example, a cleaving agent can be used to cleave at least (or at most) 5%, 10%, 15%, 20%, 25%, 30%, 35%, 40%, 45%, 50%, 55%, 60%, 65%, 70%, 75%, 80%, 85%, 90%, 95%, or 99% of the CRISPR polynucleotides in the sample. A dose of a cleaving agent can be used to cleave 100% of the CRISPR polynucleotides in the sample. The amount of cleavage can occur over at least (or at most) 1 min, 5 min, 10 min, 15 min, 30 min, 45 min, 1 hr, 2 hr, 6 hr, 12 hr, 24 hr, 48 hr, 72 hr, or 96 hr.

The release of the polynucleotide leader sequence can result in an increase in activity of a CRISPR effector protein (e.g., CRISPR enzyme, e.g., Cas9) bound to the CRISPR polynucleotide. In some cases, in a sample, release of the polynucleotide leader sequence results in at least a 0.1-fold, 0.25-fold, 0.5 fold, 0.75 fold, 1 fold, 2 fold, 5 fold, 10 fold, 50 fold, 100 fold, or 1000 fold increase in activity.

vi. Other Features

The CRISPR polynucleotide comprising a polynucleotide leader sequence can comprise a second set of one or more elements that can be subjected to a specific modification to generate a modified CRISPR polynucleotide that, when complexed with CRISPR effector protein, forms a second CRISPR complex with a lower target-specific cleavage activity. The second set of one or more elements can be a second set of one or more cleavable elements. For example, a CRISPR polynucleotide can comprise a polynucleotide leader sequence and a first set of one or more cleavable elements configured to permit release of the polynucleotide leader sequence and a second set of one or more cleavable elements configured to permit cleavage of the remaining CRISPR polynucleotide; this polynucleotide can be referred to as a CRISPR ON/OFF polynucleotide.

b. CRISPR OFF

Provided herein are CRISPR OFF polynucleotides that can be crosslinked with CRISPR effector proteins to form CRISPR OFF complexes. A CRISPR OFF polynucleotide can comprise (i) a sequence (e.g., tracrRNA sequence) configured to bind a CRISPR effector protein and (ii) a cleavable linker. In some cases, the CRISPR OFF polynucleotide further comprises a guide sequence configured to anneal to a target sequence in a target molecule. The cleavable linker can be a non-naturally occurring cleavable linker. If the CRISPR OFF polynucleotide comprises the guide sequence, the cleavable linker can be positioned 3′ of the 5′ most base in the guide sequence. The cleavable linker can be positioned within the sequence configured to crosslink the CRISPR effector protein (e.g., a tracrRNA sequence). In some cases, a base immediately 3′ and/or immediately 5′ of a cleavable linker is not annealed to another base in the CRISPR OFF polynucleotide. The cleavable linker can be a photolabile linker. The cleavable linker can be susceptible to cleavage by light, small molecule, or one or more cellular processes.

The off-target editing activity of a CRISPR effector protein complexed with a CRISPR OFF polynucleotide can be less than the off-target editing activity of a CRISPR effector protein complexed with a non-CRISPR-OFF polynucleotide, e.g., an sgRNA without one or more cleavable linkers. The off-target editing activity (e.g., as measured as described herein) can be reduced by a factor of: about 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60; at least 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60; or at most 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60. In some cases, the reduction occurs in the absence of exposure to a cleavage agent, e.g., UV light; in some cases, the reduction occurs after exposure to a cleavage agent. Complexes comprising a CRISPR effector protein complexed with a CRISPR OFF polynucleotide with a cleavable linker at positions 57 and/or 74 can have a lower off-target editing efficiency than a CRISPR effector protein complexed with an sgRNA without a cleavable linker. Complexes comprising a CRISPR effector protein complexed with a CRISPR OFF polynucleotide can have an on-target editing efficiency that is the same or is within 1%, 2%, 3%, 4%, or 5% of that of a CRISPR effector protein complexed with a non-CRISPR OFF polynucleotide. E.g., an sgRNA without a cleavable linker.

Complexes comprising a CRISPR effector protein crosslinked to the CRISPR OFF polynucleotide can be assembled. Provided herein are methods for the tunable targeting of a CRISPR complex to a target DNA. The methods can comprise cleaving the cleavable linker. Cleavage of the cleavable linker can result in a CRISPR complex with a lower target-specific cleavage activity than before the cleavage. In some cases, cleavage of the cleavable linker can cause the fragments of the CRISPR OFF polynucleotide generated by the cleaving, but not crosslinked to the CRISPR effector protein, to dissociate from the CRISPR effector protein. In some cases, cleavage of the cleavable linker renders a CRISPR complex inactive.

i. Position of the One or More Cleavable Elements

The one or more cleavable elements can be positioned 3′ of the 5′-most base (or nucleotide) in the guide sequence or 5′ of the 3′ most base (or nucleotide) in the guide sequence. The one or more cleavable elements can be positioned about 1-30 bases 3′ of the 5′ end of the crRNA or guide sequence, e.g., 2 bases, 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 21 bases, 22 bases, 23 bases, 24 bases, 25 bases, 26 bases, 27 bases, 28 bases, 29 bases, or 30 bases. The one or more cleavable elements can be positioned about 1-30 bases 3′ from the 3′ end of the crRNA sequence or guide sequence, e.g., 2 bases, 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 21 bases, 22 bases, 23 bases, 24 bases, 25 bases, 26 bases, 27 bases, 28 bases, 29 bases, or 30 bases.

The one or more cleavable elements can be positioned in the sequence of the CRISPR polynucleotide, e.g., tracrRNA sequence, configured to bind to a CRISPR effector protein (e.g., Cas9). In some cases, the one or more cleavable elements can be 1-30 bases 3′ of the 5′ end of the tracr sequence, such as 2 bases, 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 21 bases, 22 bases, 23 bases, 24 bases, 25 bases, 26 bases, 27 bases, 28 bases, 29 bases, or 30 bases. In some cases, the one or more cleavable elements can be 1-30 bases 5′ of the 3′ end of the tracr sequence, such as 2 bases, 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 21 bases, 22 bases, 23 bases, 24 bases, 25 bases, 26 bases, 27 bases, 28 bases, 29 bases, or 30 bases.

In some examples, the one or more cleavable elements can be positioned immediately 5′ or 3′ of base (or nucleotide) 56 and/or nucleotide 73 in the CRISPR polynucleotide (e.g., sgRNA), wherein the 5′-most nucleotide of the guide sequence of the CRISPR polynucleotide (e.g., sgRNA) is nucleotide 1, or replace nucleotide 57 and/or nucleotide 74. In some examples, the one or more cleavable elements can be positioned immediately 5′ or 3′ of base (or nucleotide) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 of a CRISPR polynucleotide (e.g., sgRNA), wherein the 5′-most base (or nucleotide) of the guide sequence of the CRISPR polynucleotide (e.g., sgRNA) is base (or nucleotide) 1 or replace base 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 of a CRISPR polynucleotide (e.g., sgRNA).

ii. Impact of the One or More Cleavage Elements Before Exposure to a Cleavage Agent

In some cases, a CRISPR polynucleotide (e.g., sgRNA) comprising the one or more cleavable elements and complexed with a CRISPR effector protein (e.g., Cas9) does not have a reduced activity relative to a CRISPR polynucleotide (e.g., sgRNA) without the one or more cleavable elements and complexed to a CRISPR effector protein (e.g., before exposing the CRISPR polynucleotide to a cleavage agent). In some cases, a CRISPR polynucleotide (e.g., sgRNA) comprising the one or more cleavable elements and complexed with a CRISPR effector protein (e.g., Cas9) does have a reduced activity relative to a CRISPR polynucleotide (e.g., sgRNA) without the one or more cleavable elements and complexed to a CRISPR effector protein (e.g., before exposing the CRISPR polynucleotide to a cleavage agent).

iii. Cleavage of the One or More Cleavable Elements

The one or more cleavable elements can be any cleavable element described herein. The one or more cleavable elements can be the same type of cleavable element or different types of cleavable elements. The one or more cleavable elements can be at least, or at most, 2, 3, 4, 5, 6, 7, 8, 9 or 10 cleavable elements.

The CRISPR polynucleotide (e.g., sgRNA) can be cleaved at the one or more cleavable elements while the CRISPR polynucleotide (e.g., sgRNA) is not bound to a CRISPR effector protein (e.g., Cas9). The CRISPR polynucleotide (e.g., sgRNA) can be cleaved at the one or more cleavable elements while the CRISPR polynucleotide (e.g., sgRNA) is complexed with a CRISPR effector protein (e.g., Cas9). The CRISPR polynucleotide (e.g., sgRNA) can be cleaved at the one or more cleavable elements while the CRISPR polynucleotide (e.g., sgRNA) is complexed with a CRISPR effector protein (e.g., Cas9) and bound to a target sequence. In some cases, following cleavage, one or more of the resulting fragments of the CRISPR polynucleotide (e.g., sgRNA) remains bound to the CRISPR effector protein (e.g., Cas9). In some cases, following cleavage, one or more (or all) of the resulting fragments of the CRISPR polynucleotide (e.g., sgRNA) no longer bind, or are no longer capable of binding to, a CRISPR effector protein (e.g., Cas9).

The CRISPR polynucleotide can be cleaved at the one or more cleavable elements in vitro. The CRISPR polynucleotide can be cleaved at the one or more cleavable elements in vivo. The CRISPR polynucleotide can be cleaved at the one or more cleavable elements while in a cell or organism, e.g., mouse, rabbit, goat, primate, e.g., chimpanzee, gorilla, or human.

The timing of the cleaving of the CRISPR polynucleotide (e.g., sgRNA) at the one or more cleavable elements can vary. For example, the one or more cleavable elements can be cleaved immediately after the CRISPR polynucleotide (e.g., sgRNA) is introduced into a cell or organism, or at least (or at most) 0.25, 0.5, 0.75, 1, 2, 3, 4, 5, 6, 7, 8, 9, 10, 11, 12, 13, 14, 15, 16, 17, 18, 19, 20, 21, 22, 23, 24, 48, 72, or 96 hours after introduction into a cell or organism.

A CRISPR polynucleotide (e.g., sgRNA) can be exposed to a cleavage agent once. A CRISPR polynucleotide (e.g., sgRNA) can be subjected to a cleavage agent more than once, e.g., 2 times, 3 times, 5 times, or 10 times. The CRISPR polynucleotide (e.g., sgRNA) can be exposed to more than one type of cleavage agent, e.g., at least (or at most) 2, 3, 4, 5, 6, 7, 8, 9, or 10 cleavage agents. A CRISPR polynucleotide (e.g., sgRNA) can be exposed to a cleavage agent for varying durations. For example, a CRISPR polynucleotide (e.g., sgRNA) can be exposed to a cleavage agent for 0.1 min, 0.5 min, 1 min, 2 min, 3 min, 4 min, 5 min, 10 min, 30 min, 60 min, 2 hr, 4 hr, 6 hr, 12 hr, 24 hr, 48 hr, 72 hr, or 96 hr.

In some cases, a sample comprises a plurality of CRISPR polynucleotides (e.g., sgRNAs), and a cleavage agent can be used to cleave a certain percentage of the CRISPR polynucleotides (e.g., sgRNAs). For example, a cleaving agent can be used to cleave at least (or at most) 5%, 10%, 15%, 20%, 25%, 30%, 35%, 40%, 45%, 50%, 55%, 60%, 65%, 70%, 75%, 80%, 85%, 90%, 95%, or 99% of the CRISPR polynucleotides (e.g., sgRNAs) in the sample. A dose of a cleaving agent can be used to cleave 100% of the CRISPR polynucleotides (e.g., sgRNAs) in the sample. The amount of cleavage can occur over at least (or at most) 1 min, 5 min, 10 min, 15 min, 30 min, 45 min, 1 hr, 2 hr, 6 hr, 12 hr, 24 hr, 48 hr, 72 hr, or 96 hr.

Cleavage can result in a decrease in activity of a CRISPR effector protein (e.g., CRISPR enzyme, e.g., Cas9) bound to the CRISPR polynucleotide (e.g., sgRNA). In some cases, in a sample, exposure to one or more cleavage agents results in at least a 0.1-fold, 0.25-fold, 0.5 fold, 0.75 fold, 1 fold, 2 fold, 5 fold, 10 fold, 50 fold, 100 fold, or 1000 fold decrease in activity. In some cases, in a sample, exposure to one more cleavage agents results in complete loss of activity.

c. CRISPR ON/OFF

Provided herein are CRISPR “ON/OFF” polynucleotides that can be crosslinked with CRISPR effector proteins to form CRISPR “ON/OFF” complexes. A CRISPR ON/OFF polynucleotide can comprise a guide sequence configured to anneal to a target sequence in a target molecule, a sequence (e.g., a tracrRNA sequence) configured to crosslink to a CRISPR effector protein, and (a) a first element configured to be subjected to a first specific modification that generates a first modified polynucleotide that, when crosslinked with a CRISPR effector protein, forms a first CRISPR complex with higher target-specific cleavage activity than a CRISPR complex comprising the polynucleotide that has not had been subjected to the first specific modification, and (b) a second element configured to be subjected to a second specific modification to generate a second modified polynucleotide that, when crosslinked with CRISPR effector protein, forms a second CRISPR complex with a lower target-specific cleavage activity than the first CRISPR complex. A CRISPR ON/OFF polynucleotide can comprise features of CRISPR ON polynucleotides and CRISPR OFF polynucleotides described herein.

Complexes comprising a CRISPR effector protein crosslinked to the CRISPR ON/OFF polynucleotide can be assembled. Provided herein are methods for the tunable targeting of a CRISPR complex to a target DNA. The methods can comprise subjecting the first element of the CRISPR ON/OFF polynucleotide to a first specific modification, thereby generating the first modified polynucleotide that, when crosslinked with the CRISPR effector protein, forms the first CRISPR complex with higher target-specific cleavage activity than the CRISPR complex comprising the polynucleotide that has not had been subjected to the first specific modification. The methods can further comprise subjecting the second element to the second specific modification after the subjecting the first element to the first modification, thereby forming the second modified polynucleotide that, when crosslinked with CRISPR effector protein, forms a second CRISPR complex with a lower target-specific cleavage activity than the first CRISPR complex. In some cases, the second modification can cause portions of the CRISPR polynucleotide not crosslinked to the CRISPR effector protein to fragment and/or dissociate from the CRISPR effector protein.

d. Crispr OFF Polynucleotides and Reduced Off-Target Editing

The CRISPR polynucleotide can comprise one or more modifications such that, when the polynucleotide is complexed with a CRISPR effector protein, (e.g., Cas9), to form a CRISPR complex, the CRISPR complex has a lower off-target cleavage activity than a CRISPR complex with a polynucleotide without the one or more modifications when not exposed to light. The one or more modifications can be one or more linkers described herein. The one or more modifications can be one or more cleavable linkers described herein. The one or more modifications can be one or more modifications at a 2′ position of a ribose as described herein. The one or more modifications can be one or more cleavable elements. The one or more modifications can comprise 3-(4,4′-Dimethoxytrityl)-1-(2-nitrophenyl)-propan-1-yl-[(2-cyanoethyl)-(N,N-diisopropyl)]-phosphoramidite. The CRISPR OFF polynucleotide can further comprise 2′-O-methyl analogs and 3′ phosphorothioate internucleotide linkages at the first three 5′ and 3′ terminal RNA nucleotides.

### A. Position of the One or More Modifications

The one or more modifications can be positioned 3′ of the 5′-most base (or nucleotide) in the guide sequence or 5′ of the 3′ most base (or nucleotide) in the guide sequence. The one or more modifications can be positioned about 1-30 bases 3′ of the 5′ end of the crRNA or guide sequence, e.g., 2 bases, 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 21 bases, 22 bases, 23 bases, 24 bases, 25 bases, 26 bases, 27 bases, 28 bases, 29 bases, or 30 bases. The one or more modifications can be positioned about 1-30 bases 3′ from the 5′ end of the crRNA sequence or guide sequence, e.g., 2 bases, 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 21 bases, 22 bases, 23 bases, 24 bases, 25 bases, 26 bases, 27 bases, 28 bases, 29 bases, or 30 bases.

The one or more modifications can be positioned in the sequence of the CRISPR polynucleotide, e.g., tracrRNA sequence, configured to bind to a CRISPR effector protein (e.g., Cas9). In some cases, the one or more modifications can be in a tetraloop, nexus, stem loop 1, or stem loop 2 of the CRISPR polynucleotide shown in FIG. 1. In some cases the one or more modifications can be a loop of the tetraloop, a bulge of the tetraloop, a first stem of the tetraloop, a second stem of the tetraloop, in a loop structure of the nexus, in the stem of the nexus, in a loop structure of stem loop 1, in a stem of stem loop 1, in a loop structure of stem loop 2, or in a stem of stem loop 2; examples of the tetraloop, nexus, stem loop1, and stem loop 2 are illustrated in FIG. 1. In some cases, the one or more modifications does not include sequence 5′ of the guide sequence configured to form a stem loop, e.g., with the guide sequence. In some cases, the one or more modifications can be 1-30 bases 3′ of the 5′ end of the tracr sequence, such as 2 bases, 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 21 bases, 22 bases, 23 bases, 24 bases, 25 bases, 26 bases, 27 bases, 28 bases, 29 bases, or 30 bases. In some cases, the one or more modifications can be 1-30 bases 5′ of the 3′ end of the tracr sequence, such as 2 bases, 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 21 bases, 22 bases, 23 bases, 24 bases, 25 bases, 26 bases, 27 bases, 28 bases, 29 bases, or 30 bases.

In some examples, the one or more modifications can be positioned immediately 5′ or 3′ of base (or nucleotide) 56 and/or nucleotide 73 in the CRISPR polynucleotide (e.g., sgRNA), wherein the 5′-most nucleotide of the guide sequence of the CRISPR polynucleotide (e.g., sgRNA) is nucleotide 1, or replace nucleotide 57 and/or nucleotide 74. In some examples, the one or more complex altering elements can be positioned immediately 5′ or 3′ of base (or nucleotide) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 of a CRISPR polynucleotide (e.g., sgRNA), wherein the 5′-most base (or nucleotide) of the guide sequence of the CRISPR polynucleotide (e.g., sgRNA) is base (or nucleotide) 1 or replace base 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 of a CRISPR polynucleotide (e.g., sgRNA).

### B. Impact of the One or More Modifications

In some cases, a CRISPR polynucleotide (e.g., sgRNA) comprising the one or more modifications and complexed with a CRISPR effector protein (e.g., Cas9) does not have a reduced editing activity at a target sequence relative to a CRISPR polynucleotide (e.g., sgRNA) without the one or more modifications and complexed to a CRISPR effector protein (e.g., before exposing the CRISPR polynucleotide to a cleavage agent). In some cases, a CRISPR polynucleotide (e.g., sgRNA) comprising the one or more modifications and complexed with a CRISPR effector protein (e.g., Cas9) does have a reduced editing activity at a target sequence relative to a CRISPR polynucleotide (e.g., sgRNA) without the one or more complex altering elements and complexed to a CRISPR effector protein (e.g., before exposing the CRISPR polynucleotide to a cleavage agent). In some cases, editing activity at a target sequence is reduced about 1%, 2%, 3%, 4%, 5%, 6%, 7%, 8%, 9%, or 10% or at most 1%, 2%, or 3%, 4%, 5%, 6%, 7%, 8%, 9%, or 10% relative to a standard CRISPR complex.

In some cases, a CRISPR polynucleotide (e.g., sgRNA) comprising the one or more modifications and complexed with a CRISPR effector protein (e.g., Cas9) has a reduced editing activity at an off-target sequence relative to a CRISPR polynucleotide (e.g., sgRNA) without the one or more modifications and complexed to a CRISPR effector protein (e.g., before exposing the CRISPR polynucleotide to a cleavage agent). Editing activity at an off-target sequence can be described as off-target editing. Off-target editing can be editing at a sequence that is not exactly complementary to the guide sequence of the CRISPR polynucleotide. In some cases, the editing activity at an off-target sequence is reduced about, at least, or at most 5%, 10%, 15%, 20%, 25%, 30%, 40%, 50%, 60%, 70%, 80%, 90%, or 100%. In some cases, the off-target editing activity is 0%, 1%, 5%, 10%, 15%, 20%, 25%, 30%, 40%, 50%, 60%, 70%, 80%, 90%, or 95%. In some cases, the off-target editing activity is less than 1%, 5%, 10%, 15%, 20%, 25%, 30%, 40%, 50%, 60%, 70%, 80%, 90%, or 95%. In some cases, the off-target editing activity is 0%-5%, 5%-10%, 10%-25%, 25%-50%, 50%-75%, or 75%-95%.

The off-target editing activity of a CRISPR effector protein complexed with a CRISPR OFF polynucleotide without exposure to light can be less than the off-target editing activity of a CRISPR effector protein complexed with a non-CRISPR-OFF polynucleotide, e.g., an sgRNA modified only with 2′-O-methyl analogs and 3′ phosphorothioate internucleotide linkages at the first three 5′ and 3′ terminal RNA nucleotides. The off-target editing activity of a CRISPR effector protein complexed with a CRISPR OFF polynucleotide when not cleaved (e.g., when not exposed to light) can be statistically lower, with a p-value≤0.05, ≤0.01, ≤0.005, ≤0.001, ≤0.0005, or ≤0.0001, than a CRISPR effector protein complexed with a non-CRISPR-OFF polynucleotide. The off-target editing activity (e.g., as measured as described herein) can be reduced by a factor of: about 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60; at least 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60; or at most 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60. In some cases, complexes comprising a CRISPR effector protein complexed with a CRISPR OFF polynucleotide with a modification at positions 57 and/or 74 can have a lower off-target editing efficiency than a CRISPR effector protein complexed with an sgRNA without a cleavable linker when not cleaved (e.g., when not exposed to light or another cleavage-inducing treatment). Complexes comprising a CRISPR effector protein complexed with a CRISPR OFF polynucleotide when not cleaved (e.g., when not exposed to light or another cleavage-inducing treatment) can have an on-target editing efficiency that is the same or is within 1%, 2%, 3%, 4%, or 5% of that of a CRISPR effector protein complexed with a non-CRISPR OFF polynucleotide, e.g., an sgRNA without a cleavable linker.

In some cases, the off-target editing activity is measured at one nucleic acid region. The off-target editing activity can be measured at more than one genomic region (e.g., gene). The off-target editing activity can be measured at 1, 2, 3, 4, 5, 6, 7, 8, 9, 10, 25, 50, 75, or 100 genomic regions (e.g., genes). The off-target editing activity can be measured at 1, 2, 3, 4, 5, 6, 7, 8, 9, 10, 25, 50, 75, 100, 1000, or 10,000 genomic regions (e.g., genes). The off-target editing activity can be measured at most 1, 2, 3, 4, 5, 6, 7, 8, 9, 10, 25, 50, 75, 100, 1000, or 10,000 genomic regions (e.g., genes).

The off-target editing activity can be measured by analyzing nucleic acid molecules from a cell contacted by CRISPR complex. The measurement can be made using nucleic acid molecules extracted from the cells, about, or at most 30 minutes, 1 hours, 2 hours, 5 hours, 10 hours, 12 hours, 24 hours, 36 hours, 48 hours, 60 hours, 72 hours, 4 days, 5 days, or 6 days after transfection. The CRISPR complex can be introduced into the cell by transformation. The nucleic acid molecules can be analyzed by, e.g., sequencing, PCR, mass spectrometry, southern blot, etc. The off-target editing can be visualized, e.g., by presenting data in, e.g., graph, e.g., scatterplot.

CRISPR complexes comprising a CRISPR polynucleotide can be used to reduce off-target editing as compared to Cas9 complexed with an sgRNA without a modification as described herein. Off-target editing can be determined using ICE (Inference of CRISPR Editing) to measure the amount of gene editing by analyzing Sanger sequencing traces and mapping level of sequence breakdown to determine indel formation frequencies, as described in Hsiau et al. “Inference of CRISPR Edits from Sanger Trace Data”, Jan. 14, 2019 bioRxiv or deep-sequencing techniques as described in Tsai et al. “GUIDE-seq enables genome-wide profiling of off-target cleavage by CRISPR-Cas nucleases”, Nature Biotechnology 33, 187-197 (2015). Off target editing sites can have sequences that have a high percent sequence identity to the target sequence. The sequence identity can be less than or equal to 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 310% or 30%. Off target editing sites can have sequences that are close in proximity to a PAM region, for example mismatches between the guide RNA and DNA may be tolerated at the 5′ end of the protospacer (distal to the PAM) to produce an off-target edit. Those of skill in the art readily understand how to determine sequence identity between two nucleic acids. For example, the sequence identity can be calculated after aligning the two sequences so that the sequence identity is at its highest level. Another way of calculating sequence identity can be performed by published algorithms. Optical alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.; the BLAST algorithm of Tatusova and Madden FEMS Microbiol. Lett. 174: 247-250 (1999) available from the National Center for Biotechnology Information (http://www.ncbi.nlm.nih.gov/blast/b12seq/b12.html), or by inspection.

e. Cleavable Elements

The one or more cleavable elements can be any cleavable element described herein.

i. Types of Cleavable Elements

The cleavage property of the CRISPR polynucleotide can be altered by a cleavable element that can alter the propensity of cleavage of the CRISPR polynucleotide at the point of its incorporation, under appropriate conditions. A “cleavable element” can comprise natural nucleotides or one or more modified nucleotides. The cleavable element can be incorporated into the CRISPR polynucleotide (e.g., sgRNA) during nucleic acid synthesis.

Two or more cleavable elements in a CRISPR polynucleotide can have different cleavage characteristics, e.g., the two or more cleavable elements, when incorporated into a CRISPR polynucleotide (e.g., sgRNA), can be selectively cleaved in each other's presence by using different agents and/or reaction conditions.

As used herein, the terms “cleaving,” “cleaved” and “cleavage” can all relate to the scission of the CRISPR polynucleotide (e.g., sgRNA) substantially at each point of occurrence of a cleavable element in the CRISPR polynucleotide (e.g., sgRNA).

The cleavage can be initiated by an agent. The agent can be, e.g., a chemical entity or physical force that causes the cleavage of a cleavable element. The agent can be a chemical or combination of chemicals, a biomolecule or combination of biomolecules, normal or coherent (laser) visible or ultraviolet (UV) light, heat or other forms of electromagnetic energy. In some cases, a combination of agents, e.g., two or more agents, can be used simultaneously or sequentially to cleave a CRISPR polynucleotide (e.g., sgRNA). By simultaneously is meant a CRISPR polynucleotide (e.g., sgRNA) can be exposed to the two or more agents at the same time, although the two or more agents can react with the CRISPR polynucleotide (e.g., sgRNA) one at a time. By sequentially is meant that the CRISPR polynucleotide (e.g., sgRNA) can be contacted with one agent and then a second agent at a later time.

A CRISPR polynucleotide (e.g., sgRNA) can comprise more than one type of cleavable element. In some examples, the first cleavable element and the second cleavable element have the same cleavage characteristics. In some examples, the second cleavable element has different cleavage characteristics than the first cleavable element. For example, the first cleavable element can be a photocleavable linker and the second cleavable element can be susceptible to cleavage by a chemical nuclease. In another example, the first cleavable element can be susceptible to cleavage by a chemical nuclease, and the second cleavable element can be engineered to be photocleavable allowing orthogonal treatment regimens to be applied. In some cases, the same cleavable element can have more than one type of cleavage characteristic. The first and second cleavable element can be any cleavable element described herein.

A cleavable element (e.g., cleavable linker) can refer to an entity that can connect two or more constituents of a CRISPR polynucleotide (e.g., sgRNA) that renders the CRISPR polynucleotide (e.g., sgRNA) susceptible to cleavage under appropriate conditions. For instance, the appropriate conditions can be exposure to UV light. The cleavable linker can comprise one or more modified or unmodified nucleotides, which are susceptible to scission under the appropriate conditions.

The cleavable linker can comprise a modified internucleoside linkage. The modified internucleoside linkage can be an internucleotide linkage that has a phosphorus atom or those that do not have a phosphorus atom. Internucleoside linkages containing a phosphorus atom therein include, for example, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, P-ethyoxyphosphodiester, P-ethoxyphosphodiester, P-alkyloxyphosphotriester, methylphosphonate, thionophosphoramidates, thionoalkylphosphonates, thionoalkylphosphotriesters, selenophosphates and boranophosphates, and nonphosphorus containing linkages, e.g., acetals and amides, such as are known in the art, having normal 3′-5′ linkages, 2′-5′ linked analogs of these, and those having inverted polarity wherein one or more internucleotide linkages is a 3′ to 3′, 5′ to 5′ or 2′ to 2′ linkage. Polynucleotides having inverted polarity can comprise a single 3′ to 3′ linkage at the 3′-most internucleotide linkage i.e. a single inverted nucleoside residue which may be abasic (the nucleobase is missing or has a hydroxyl group in place thereof).

Non-phosphorus containing internucleoside linkages include short chain alkyl, cycloalkyl, mixed heteroatom alkyl, mixed heteroatom cycloalkyl, one or more short chain heteroatomic and one or more short chain heterocyclic. These internucleoside linkages include but are not limited to siloxane, sulfide, sulfoxide, sulfone, acetyl, formacetyl, thioformacetyl, methylene formacetyl, thioformacetyl, alkeneyl, sulfamate; methyleneimino, methylenehydrazino, sulfonate, sulfonamide, amide and others having mixed N, O, S and CH2 component parts. Other modified internucleoside linkages that do not contain a phosphorus atom therein include, —CH₂—NH—O—CH₂—, —CH₂—N(CH₃)—O—CH₂— (known as a methylene (methylimino)backbone), —CH₂—O—N(CH₃)—CH₂—, —CH₂—N(CH₃)—N(CH₃)—CH₂— and —O—N(CH₃)—CH₂—CH₂—.

The cleavable linker can be non-nucleotide in nature. A “non-nucleotide” can refer to any group or compound that can be incorporated into a polynucleotide chain in the place of one or more nucleotide units, including either sugar and/or phosphate substitutions. The group or compound can be abasic in that it does not contain a commonly recognized nucleotide base, such as adenosine, guanine, cytosine, uracil or thymine, for example at the C1 position of the sugar.

Non-nucleotidic linkers can be e.g., abasic residues (dSpacer), oligoethyleneglycol, such as triethyleneglycol (spacer 9) or hexaethylenegylcol (spacer 18), or alkane-diol, such as butanediol. The spacer units can be preferably linked by phosphodiester or phosphorothioate bonds. The linker units can appear just once in the molecule or can be incorporated several times, e.g., via phosphodiester, phosphorothioate, methylphosphonate, or amide linkages. Further preferred linkers are alkylamino linkers, such as C3, C6, C12 aminolinkers, and also alkylthiol linkers, such as C3 or C6 thiol linkers. In some examples, heterobifunctional and homobifunctional linking moieties can be used to conjugate peptides and proteins to nucleotides. Examples include 5′-Amino-Modifier C6 and 3′-Amino-Modifier C6 reagents.

ii. Methods of Cleaving Cleavable Elements

The cleavable element can be cleaved by any suitable method, including exposure to acid, base, nucleophile, electrophile, radical, metal, reducing or oxidizing agent, light, temperature, enzymes, small molecule, nucleic acid, protein, etc. In some examples, the cleavable element (e.g., cleavable linker) is susceptible to cleavage by a cellular process or byproduct thereof. The cellular process can involve enzyme, second messenger molecules, metabolites, proteins, and free radicals.

iii. Photolabile Groups

The cleavable element can be a photolabile group. The photolabile group can be introduced into the CRISPR polynucleotide by phosphoramidite chemistry. If a photolabile group is used to crosslink, the photolabile group can be the same or different from the photolabile cleavable element. If the photolabile group used to crosslink is different from the photolabile group used to cleave, a different wavelength can be used to activate cleavage than is used to activate crosslinking. Two or more photolabile elements in a CRISPR polynucleotide can have different activation characteristics, e.g., the two or more elements, when incorporated into a CRISPR polynucleotide can be selectively activated in each other's presence by using different agents and/or reaction conditions.

Selective reaction of PC-aminotag phosphoramidites with the free 5′-OH group of a growing oligonucleotide chain, followed by cleavage from the support and deprotection, can result in the introduction of a phosphodiester group linked to a primary aliphatic amino group through a photocleavable linker. This amino group can then be used to introduce a variety of photocleavable markers through a postsynthetic modification reaction with amine reactive reagents (Olejnik J et. al, Nucleic acids research. 1998; 26:3572-6. For example, a CRISPR polynucleotide can comprise a photocleavable aliphatic group linking two nucleotides (e.g., nucleotide 53 and nucleotide 54) in the CRISPR polynucleotide, and the CRISPR polynucleotide can be exposed to UV light, resulting in a break in the CRISPR polynucleotide (e.g., between nucleotide 53 and 54). In other examples, a photocleavable aminotag phosphoramidite can be positioned in a CRISPR polynucleotide between the polynucleotide leader sequence and the guide sequence, and UV light can be used to initiate cleavage at the photocleavable aminotag phosphoramidite, thereby separating the polynucleotide leader sequence. An example of a photocleavable linker that can be used to initiate cleavage of the CRISPR polynucleotide can be 3-(4,4′-Dimethoxytrityl)-1-(2-nitrophenyl)-propan-1-yl-[(2-cyanoethyl)-(N,N-diisopropyl)]-phosphoramidite. For example, a CRISPR polynucleotide can comprise a photocleavable aliphatic group linking two nucleotides (e.g., nucleotide 53 and nucleotide 54) in the CRISPR polynucleotide, and the CRISPR polynucleotide can be exposed to visible light, resulting in a break in the CRISPR polynucleotide (e.g., between nucleotide 53 and 54). In other examples, a photocleavable coumarin photolinker can be positioned in a CRISPR polynucleotide between the polynucleotide leader sequence and the guide sequence, and visible light can be used to initiate cleavage at the photocleavable coumarin photolinker, thereby separating the polynucleotide leader sequence. An example of a photocleavable linker that can be used to initiate cleavage of the CRISPR polynucleotide can be a coumarin linker. Other methods of introducing photocleavable linkers into polynucleotide sequences have been described, e.g., in US Patent Applications: US20080227742A1, US20100022761A1, U.S. Pat. No. 7,897,737B2, the contents of which have been referenced here in their entirety.

iv. Ribonuclease Based Cleavage

In some examples, the one or more cleavable elements comprise a cleavage site for an endoribonuclease, e.g., an endoribonuclease which cleaves RNA at or within a defined ribonucleotide sequence motif. For example, the cleavable element can comprise a cleavage site recognized by a sequence-specific endoribonuclease. The endoribonuclease can be naturally occurring or engineered. In some examples, the endoribonuclease can be specific for single stranded RNA, double stranded RNA or a nucleotide sequence formed by a DNA:RNA hybrid. In some examples, the sequence-specificity of the endoribonuclease can be engineered by fusion with oligonucleotides or by fusion with other protein domains. For example, a sequence specific endoribonuclease enzyme can be engineered by fusing two functionally independent domains, a RNase HI, that hydrolyzes RNA in DNA-RNA hybrids in processive and sequence-independent manner, and a zinc finger that recognizes a sequence in DNA-RNA hybrids. In another conjugation of an antisense oligodeoxynucleotide to ribonuclease H can result in sequence-specific cleavage. See e.g., Sulej et. al, Nucleic acids research. 2012; 40(22):11563-70 and Fukuma et. al, Bioconjugate chemistry. 2003; 14(2):295-301. In some cases, the cleavable element can be capable of recruiting RNase H1 to cleave double stranded regions of the CRISPR polynucleotide. (See, e.g., U.S. Pat. No. 5,849,902).

The cleavable element can comprise a cleavage site recognized by a sequence-specific ssRNA endoribonuclease such as the excised IVS rRNA portion of the Tetrahymena thermophila as described, e.g., in Zaug et. al, Biochemistry 1988; 27, 25, 8924-8931. In other examples the cleavable element can comprise one or more cleavage sites recognized by sequence-specific ssRNA endoribonuclease Cas2 as described, e.g., in Beloglazova et. al, J Biol Chem. 2008; 283(29): 20361-20371. In other examples the cleavable element can comprise one or more preferred sites in dsRNA recognized by RNase Mini-III from Bacillus subtilis, e.g., as discussed in Glow et. al, Nucleic Acids Res. 2015; 43 (5) 2864-73. In other examples, Short oligonucleotides can be used as external guide sequences (EGSs) to direct site-specific cleavage of the CRISPR polynucleotide by human RNase P. For example, 13-mer EGSs targeted to the 2.1-kb surface antigen mRNA of hepatitis B virus (HBV) were capable of inducing cleavage of the HBV RNA by RNase P. (See Werner M et. al, RNA. 1998; 4(7):847-55. The endoribonuclease can be a member of the sequence or structure specific endoribonuclease Cas6 superfamily, e.g., Cas6A (e.g. Hong Li (2015), Structure, January 6; 23(1): 13-20). The endoribonuclease can be Csy4, also known as Cas6f. The ssRNA endoribonuclease can belong to the Cas13 family of CRISPR endoribonuclease or derivatives thereof. The endoribonuclease can be Cpf1 or a Cas5d enzyme, which can process pre-creRNA transcripts (Zetsche, B. et al. (2016), “Multiplex gene editing by CRISPR-Cpf1 using a single crRNA array”, Nature Biotechnology (2016) doi: 10.1038/nbt.3737).

The cleavable element can be an element that is cleavable by ribozymes, e.g. the hammerhead ribozyme, Hepatitis delta virus ribozyme etc. The ribozymes can be naturally occurring or can be engineered to be a trans-acting ribozymes by separation into ‘catalyst’ and ‘substrate’ strands as discussed, e.g., in Levy et. al, RNA 2005. 11: 1555-1562. In some cases, two ribozymes can be used in concert to allow cleavage after a desired target sequence. In some cases, alternative artificial ribozyme-protein complexes that function in different cellular compartments can be designed by the use of localizing determinants for delivering a ribozyme to a specific subcellular site or for targeting a specific type of RNA as shown in Samarsky et. al, Proc Natl Acad Sci USA. 1999; 96(12): 6609-6614. In some cases, use of the ribozyme can involve binding of an exogenous small molecule for activity, e.g., glmS ribozyme.

In some examples, the activity of the ribozyme can be further tuned to be ligand-controlled by coupling with an aptamer. The aptamer can be chosen based on its ability to bind a ligand or otherwise “sense” a change in environment (such as pH, temperature, osmolarity, salt concentration, etc) in a manner directly coupled through an information transmission domain to loop I and/or loop II. The ligand can, for example, be a protein, nucleotide or small molecule ligand. The binding of the ligand to the aptamer can causes a change in the interaction of the information transmission domain with one or more of the loop, the stem or the catalytic core such that the ribozyme activity can be modulated dependent upon the presence or absence of the ligand as described, e.g., in U.S. Pat. No. 8,603,996B2.

Cleavage of the cleavable elements of the CRISPR polynucleotide (e.g., sgRNA) can be induced at a desired time independently; for example, a genetically-coded endoribonuclease can be activated within the host cells. A vector or plasmid encoding the endoribonuclease can be transfected into the cell at a desired time. One or more endoribonucleases can be under the control of one or more independent promoters. One or more of the promoters can be activated at desired times.

v. Antisense Oligonucleotides

The one or more cleavable elements of the CRISPR polynucleotide can be designed to allow the binding of an anti-sense oligonucleotide. The antisense oligonucleotide can be a single-stranded DNA (ssDNA) oligonucleotide. The ssDNA oligonucleotide can hybridize to single stranded RNA sequence in the CRISPR polynucleotide, and RNAse H can be used to cleave RNA of the DNA:RNA hybrid. With regard to the cleavable element (e.g., RNA loop of a stem loop in the CRISPR polynucleotide) to which the antisense oligonucleotide can bind, the cleavable element (e.g., loop of a stem loop) can be about 6 to about 40 nucleotides in length. The antisense oligonucleotide can be about 12 to about 16 nucleotides in length, or about 12 to about 25 nucleotides, or about 10 to about 30 nucleotides in length. The degree of complementarity between the antisense oligonucleotide and the cleavable element (e.g., loop of a stem loop) of the CRISPR polynucleotide can be at least 80%, 85%, 90%, 95%, 98%, 99%, or 100%. An antisense oligonucleotide whose sequence is fully or partially complementary to the cleavable element can be produced within the host cell or introduced into the host cell. Antisense oligonucleotides can be transfected into cells using polyethyleneimine (PEI) or other known transfection methods.

The one or more cleavable element of the CRISPR polynucleotide can comprise a miRNA responsive element. The length of the miRNA responsive element can be between about 15 to about 30 nucleotides, e.g., about 20 to about 25 nucleotides in length. The length of the miRNA can be about 20 to about 24 nucleotides, e.g., about 21 to about 23 nucleotides, e.g., about 22 nucleotides in length. The degree of sequence complementarity between the miRNA and the miRNA responsive element in the CRISPR polynucleotide can be at least 80%, 85%, 90%, 95%, 98%, 99%, or 100%.

The cleavable element can comprise a miRNA response element (MRE), and a miRNA that is capable of binding to the MRE can be produced within the host cell or introduced into the host cell. The miRNA can be present in the form of an miRISC complex which can target the MRE and cleave the first cleavable element.

vi. Site-Specific Chemical Nucleases

Specific cleavage of the CRISPR polynucleotide can be achieved by a chemical compound that has been designed to possess site-specific nuclease activity.

The chemical nuclease can be designed to have sequence-specific affinity to a CRISPR polynucleotide, e.g., CRISPR ON polynucleotide, CRISPR OFF polynucleotide, or CRISPR ON/OFF polynucleotide described herein. For example, RNA cleaving tris(2-aminobenzimidazoles) can be attached to DNA oligonucleotides or 2′-O-methyloligoribonucleotide via disulfide or amide bonds to form organocatalytic nucleases showing RNA substrate and site selectivity (see e.g., Gnaccarini et. al, J. Am. Chem. Soc., 2006, 128 (24), pp 8063-8067]. In other examples, the site-specificity of the chemical RNAse (e.g., 1,10-phenanthroline moiety, neocuprine Zn (II), neamine) for the CRISPR polynucleotide can be achieved through the use of peptide nucleic acids (PNA), e.g., polyamide nucleic acid.

The site-specificity of the chemical RNAse (e.g., diethylenetriamine moiety) for the CRISPR polynucleotide can be achieved by a combined use of anti-sense oligonucleotides, peptides proteins or PNAs. In some examples, RNA-binding proteins can be chemically converted to sequence-specific nucleases by covalent attachment to a coordination complex, such as 1,10-phenanthroline-copper complex. See e.g., Chen et. al, Sigman D S. Science. 1987; 237(4819):1197-201. In another example, site-specific cleavage of CRISPR polynucleotide can be achieved by the conjugation of Bleomycin-Fe (II) with EDTA or an oligonucleotide to form an artificial nuclease with specificity for the CRISPR polynucleotide.

Examples of chemical nucleases include 1,10-phenanthrolinecopper (Sigman et al., 1993), ferrous-ethylenediaminetetraacetic acid (EDTA), macrocylic lanthanide complexes, metalloporphyrins, metallic complexes of salens, uranyl acetat, octahedral metal complexes of rhodium (III), benzene diazonium teetrafluoroborate, aliphatic monoamines-, diamines- and polyamines, aminoglycosides such as neomycin B and copper (II) aminoglycoside complexes etc. In some cases, the chemical nucleases can target the sugar moiety of nucleosides and catalyze oxidative cleavage by extracting a hydrogen atom from the sugar at the cleavage site.

vii. Photochemical Cleavage

In some examples, photocaging groups can be used to render further control on the activity of the agent used for the cleavage of the CRISPR polynucleotide. For example, photolysis of photoactivatable or “caged” probes can be used for controlling the release of site-specific chemical nucleases described in this disclosure. In another example, a photocaging group can be used to block cleavage by a ribonuclease or restriction enzyme of the CRISPR polynucleotide, until released by photolysis, e.g., as shown in Bohacova et. al, Biomol. Chem., 2018. 16, 1527. In another example, a photocaging group on one or more of the nucleotides in the CRISPR polynucleotide can be used to mask the recognition sequence for an anti-sense nucleotide, until release by photolysis, thereby initiating cleavage of the CRISPR polynucleotide. In another example, the photocaging group can be attached to the cleavage agent, such as the anti-sense oligonucleotide, which upon photolysis, becomes available for binding to the CRISPR polynucleotide and initiating the formation of a RISC complex. In another example, the photocaging group can be used to mask a ‘miRNA response element’ for cleavage of the CRISPR polynucleotide until release by photolysis. In other aspects, without limitation, photocaging groups can be used with orthogonal treatment regimens for the cleavage of multiple cleavage elements with different cleavage characteristics.

Photocaging groups can be used for ‘tagging’ the cleavage reaction, wherein the tag can be amenable for detection and/or quantification by one or more methods. For example, a 2-nitro-benzyl based photocleavable group can be labeled further with a dye that is released upon photolysis, and can be used as a detectable marker for the ‘efficiency’ of activation of the CRISPR ON polynucleotide or for the deactivation of the CRISPR OFF polynucleotide etc. In another example, the ribonuclease protein that binds to the cleavable element of CRISPR polynucleotide can be tagged upon initiation of the ‘cleavage event’ by the release of a ‘fluorescent tag’ from photocaged nucleotide that was incorporated into the cleavable element, wherein measurement of the fluorescent tag can be a surrogate marker for the cleavage of the CRISPR polynucleotide.

Examples of photocaging groups that can be synthetically incorporated into the CRISPR polynucleotide include ortho-nitrobenzyl based caging groups that can by linked to a heteroatom (usually O, S or N) as an ether, thioether, ester (including phosphate or thiophosphate esters), amine or similar functional group by methods known in the art. Examples of 2-nitrobenzyle based caging groups include α-carboxy-2-nitrobenzyl, 1-(2-nitrophenyl)ethyl, 4,5-dimethoxy-2-nitrobenzyl, 1-(4,5-dimethoxy-2-nitrophenyl)ethyl, 5-carboxymethoxy-2-nitrobenzyl, nitrophenyl etc. Other examples of photoremovable protecting groups, include benzyloxycarbonyl, 3-nitrophenyl, phenacyl, 3,5-dimethoxybenzoinyl, 2,4-dinitrobenzenesulphenyl, Ethedium Monoazide, Bimane Azide and their respective derivatives.

Photolabile linkers described herein can be represented as several mesomeric forms. Where a single structure is drawn, any of the relevant mesomeric forms are intended. The coumarin linkers described herein represented by a structural formula can be shown as any of the related mesomeric forms. Exemplary mesomeric structures are shown below for Formula

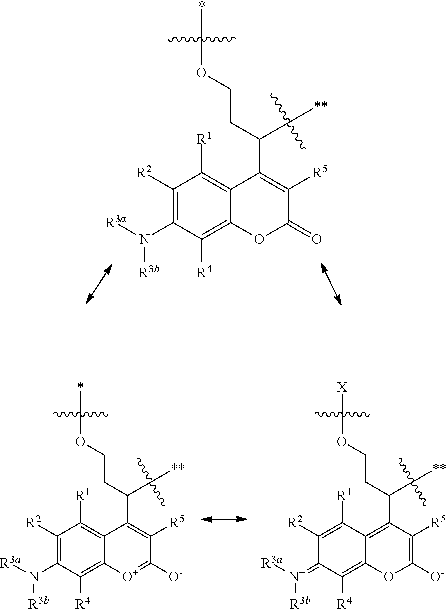

Photolabile protective groups can be attached to the hydroxy and phosphate or nucleobase in nucleosides and nucleotides. For example, photocaged derivatives of 2′-deoxy-5-(hydroxymethyl) uridine nucleoside, mono- and triphosphates protected by 2-nitrobenzyl-, 6-nitropiperonyl- and anthryl-9-methyl groups can be their enzymatically incorporated into the polynucleotide, e.g., as described in Bohacova et. al, Org. Biomol. Chem., 2018, 16, 152. Photocleavage can occur through a variety of mechanisms such as hydrogen bond abstraction from sugar ring, direct electron transfer from the base to the photo excited cleaver or singlet oxygen production by transfer of energy from the photocleavage and formation of adducts.

viii. Cleavage of Cleavable Element

The cleavable element(s) of the two or more (e.g., 2, 3, 4, 5, 6, 7, 8, 9 or 10) CRISPR polynucleotides can be cleaved by the same cleaving moiety. The cleavage of the two or more (e.g. 2, 3, 4, 5, 6, 7, 8, 9 or 10) different CRISPR polynucleotides can be induced by different external factors. The cleavage inducing agent can be electromagnetic radiation. The cleavage inducing agent can be a particular wavelength of light in the visible spectrum. The cleavage element can be cleaved by UV light.

The wavelength of the light can range from 220-465 nm. The intensity of light in the exposure protocol can be about 5, 10, 15, 20, 25, 35, 40, 50, 70, 90, 110, 120, 140, 160, 175, 190, 200, 220, 240, 260 280, 300, 320, 340, 360, 380, 400, 420, 440, 460, 480, 500, 520, 540, 560, 580, 600, 620, 650, 675, 700, 720, 745, 765, 790, 810, 830, 850, 870, 900, 920, 945, 965, 985, 1000, 1025, 1050, 1080, 1100, 1125, 1150, 1175, 1200, 1240, 1275, 1290, 1320, 1350, 1380, 1400, 1420, 1450, 1470, 1490, 1520, 1540, 1560, 1600, 1630, 1650, 1670, 1700, 1720 or 1750 mW/cm². The intensity of light in the exposure protocol can range from about 70 mW/cm² to 100 mW/cm², 80 mW/cm² to 110 mW/cm², 90 mW/cm² to 120 mW/cm², 100 mW/cm² to 130 mW/cm², 110 mW/cm² to 140 mW/cm², 120 mW/cm² to 150 mW/cm², 130 mW/cm² to 160 mW/cm², 140 mW/cm² to 170 mW/cm², 150 mW/cm² to 180 mW/cm², 160 mW/cm² to 190 mW/cm², 170 mW/cm² to 200 mW/cm², 180 mW/cm² to 210 mW/cm², 190 mW/cm² to 220 mW/cm², 200 mW/cm² to 230 mW/cm², 210 mW/cm² to 240 mW/cm², 220 mW/cm² to 250 mW/cm², 230 mW/cm² to 260 mW/cm², 240 mW/cm² to 270 mW/cm², 250 mW/cm² to 280 mW/cm², 260 mW/cm² to 290 mW/cm², or 270 mW/cm² to 300 mW/cm². The wavelength of the light can range from about 320 nm to about 390 nm. The wavelength of the light can range from about 320 nm to 425 nm, 320 nm to 420 nm, 420 nm to 520 nm, 520 nm to 620 nm. 420 nm to 700 nm. The wavelength of light can be greater than about 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, or 700 nm. The wavelength of light can be less than about 700 nm, 690 nm, 680 nm, 670 nm, 660 nm, 650 nm, 640 nm, 630 nm, 620 nm, 610 nm, 600 nm, 590 nm, 580 nm, 570 nm, 560 nm, 550 nm, 540 nm, 530 nm, 520 nm, 510 nm, 500 nm, 490 nm, 480 nm, 470 nm, 460 nm, 450 nm, 440 nm, 430 nm, or 425 nm. The wavelength of light can range from about 420 nm to 430 nm, 430 nm to 440 nm, 440 nm to 450 nm, 450 nm to 460 nm, 460 nm, to 470 nm, 470 nm to 480 nm, 480 nm to 490 nm, 490 nm to 500 nm, 500 nm to 510 nm, 510 nm to 520 nm, 520 nm to 530 nm, 530 nm to 540 nm, 540 nm to 550 nm, 550 nm to 560 nm, 560 nm to 570 nm, 570 nm to 580 nm, 580 nm to 590 nm, 590 nm to 600 nm, 600 nm to 610 nm, 610 nm to 620 nm, 620 nm to 630 nm, 630 nm to 640 nm, 640 nm to 650 nm, 650 nm to 660 nm, 660 nm to 670 nm, 670 nm to 680 nm, 680 nm to 690 nm, or 690 nm to 700 nm. The power wattage of the light used in the exposure protocol can be about 50, 70, 80, 90, 100, 120, 140, 160, 175, 190, 210, 230, 250, 270, 290, 310, 330, 250, 370, 390, 420, 4450, 480, 500, 530, 550, 570, 600, 620, 650, 670, 700, 720, 750, 770, 800, 820, 850, 870, 900, 920, 950, 970, 1000, 1020, 1050, 1070, 1100, 1120, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400 4500, 4600, 4700, 4800, 4900, 5000, 5100, 5200, 5300, 5400, 5500, 5600, 5700, 5800, 5900, or 6000 W, as measured by an OAI 306 UV power meter.

The duration of exposure can be from 1 second to 30 minutes. The duration of exposure can be from 1 second to 30 seconds, 30 seconds to 60 seconds, 1 min to 5 min, 5 min to 10 min, 10 min to 20 min, 20 min to 30 min, 30 min to 40 min, 40 min to 50 min, or 50 min to 1 hr. The duration of exposure can be greater than about one hour, 50 min, 40 min, 30 min, 20 min, 10 min, 5 min, 1 min, 30 seconds, or one second. The duration of exposure can be less than about two seconds, 30 seconds, 1 min, 5 min, 10 min, 20 min, 30 min, 40 min, 50 min, or 1 hour. The exposure protocol can comprise continuous exposure or pulsed exposure or both. The pulse exposure can be uniform or of varying durations.

The light source can be a broad spectrum light that has been filtered through a bandpass filter. The bandpass filter can be a 345 nm bandpass filter. The bandpass filter can be a 420 nm long pass filter. The light source can be an ultraviolet (UV) light. The light source can be a LED. The LED can emit ultraviolet light. The LED can emit visible light. The LED can emit infrared light.

### B. Modified CRISPR Effector Proteins for Conjugation

In some cases, CRISPR effector proteins are modified to facilitate conjugation to a CRISPR polynucleotide. The CRISPR polynucleotide can be modified to facilitate conjugation to a CRISPR effector protein. The CRISPR polynucleotide can comprise a CRISPR ON polynucleotide sequence, a CRISPR OFF polynucleotide sequence, a CRISPR ON/OFF polynucleotide sequence, or CRISPR polynucleotides comprising one or more modifications that, when complexed with a CRISPR enzyme, have reduced off-target editing activity, described herein. Both the CRISPR effector protein and the CRISPR polynucleotide can be modified to facilitate conjugation to a CRISPR effector protein. For example, the CRISPR effector protein can be modified with unnatural amino acids to facilitate cross linking to the CRISPR polynucleotide. In some cases, both the CRISPR polynucleotide (e.g., sgRNA) and the CRISPR effector protein are modified to facilitate conjugation. In some cases, only the CRISPR effector protein comprises a cross-linker, and the CRISPR polynucleotide does not comprise a cross-linker. In some cases, the CRISPR polynucleotide comprises a cross-linker and the CRISPR effector protein does not comprise a cross-linker.

As described below, the CRISPR effector protein can be modified either by the inclusion of one or more unnatural amino acids or by fusion (e.g., expression of a fusion) of the CRISPR effector protein with an amino acid sequence, such as a SNAP fusion protein, designed to facilitate binding to another molecule.

The CRISPR effector protein, e.g., Cas9, can comprise one or more mutations (and hence nucleic acid molecule(s) coding for same can have mutation(s)). The one or more mutations can be artificially introduced mutations and can be one or more mutations in a catalytic domain. Examples of catalytic domains with reference to a Cas9 enzyme can be RuvC I, RuvC II, RuvC III and HNH domains. The one or more mutations can render the one or more catalytic domains of Cas9 inactive. The one or more mutations can reduce the catalytic activity of Cas 9 0.1-fold, 0.25-fold, 0.5-fold, 0.75-fold, 1-fold, 2-fold, 5-fold, 10-fold, 50-fold, 100-fold, or 1000-fold. In some cases, the one or more mutations can increase the catalytic activity of Cas9 0.1-fold, 0.25-fold, 0.5-fold, 0.75-fold, 1-fold, 2-fold, 5-fold, 10-fold, 50-fold, 100-fold, or 1000-fold.

The CRISPR polynucleotide can be modified with a cell penetrating RNA aptamer. The cell penetrating RNA aptamer can improve the effective delivery of the CRISPR polynucleotide to a cell. The RNA aptamer can bind to a cell surface receptor and promote the entry of CRISPR polynucleotide into a cell. The cell penetrating aptamer can be designed to target a specific cell receptor in order to mediate cell-specific delivery.

### 1. Types of Modifications for Conjugation

CRISPR effector protein can be modified by the inclusion of unnatural amino acids. Unnatural amino acids include photo labile unnatural amino acids, e.g., photo labile unnatural amino acid cross linkers, e.g., p-azido-L-phenylalanine (AzF) or p-benzoyl-L-phenylalanine (BzF), can be used to form crosslinks in bio conjugation. Upon excitation at 350-360 nm, benzophenones, e.g., BzF, can preferentially react with otherwise inactivated carbon-hydrogen bonds on exposed functional groups located on the CRISPR polynucleotide. In some cases, benzophenones do not photo dissociate and their photo excited triplet state readily relaxes in the absence of a suitable carbon-hydrogen bond with which to react, thereby benzophenone can be a more forgiving reagent than other conjugating reagents. AzF can generate reactive nitrenes upon exposure to ultraviolet light which can be used to link to the CRISPR polynucleotide.

The CRISPR effector protein can be fused to another protein, e.g., a SNAP protein. Configurations can involve the covalent attachment of a DNA repair template to the SNAP protein through a BG (06-benzylguanine) linker as a method of keeping a DNA repair template close to the CRISPR complex and/or the attachment of the CRISPR polynucleotide, e.g., sgRNA, with a linker nucleotide sequence to attach to the SNAP protein through the use of a BG linker and a RNA aptamer as described below.

The CRISPR effector protein can be modified with a SNAP protein fusion to facilitate linking of the CRISPR polynucleotide (e.g., sgRNA) to the CRISPR effector protein. For example, a vector can be used to express a fusion protein comprising a CRISPR effector protein and a SNAP protein followed by an arm region. The arm region further comprises a series of amino acids configured for flexibility. The arm region can be further configured to link to a benzyl guanine modified polynucleotide. The SNAP protein region can be located at the N-terminus of the CRISPR effector protein. At the N-terminus of the SNAP protein can be the arm region. The CRISPR polynucleotide can be modified with a benzyl-guanine-binding RNA aptamer (as described, e.g., by Carrocci and Hoskins, Evolution and Characterization of a Benzylguanine-Binding RNA Aptamer, Chem Commun (Camb). 2016 Jan. 11; 52(3): 549-552. doi:10.1039/c5cc07605f). Once the CRISPR polynucleotide is bound with a benzyl-guanine binding RNA aptamer, the CRISPR polynucleotide can be covalently bonded to the arm region of the fusion protein. The flexibility of the arm region can allow the CRISPR polynucleotide to complex with the CRISPR effector protein region of the fusion protein. Alternatively, the CRISPR polynucleotide can be complexed prior to attachment to the arm region of the fusion protein.

C. Crispr Complexes with High Affinity Binding of Crispr Effector Protein and Polynucleotide

Provided herein is a CRISPR complex comprising (a) the polynucleotide comprising a sequence designed to anneal to a target nucleic acid sequence and a sequence designed to bind a CRISPR effector protein, and an activity modulating polynucleotide sequence (e.g. CRISPR ON, CRISPR OFF, or CRISPR ON/OFF, described herein); and (b) a CRISPR effector protein wherein an equilibrium dissociation constant (K_{d}) for the polynucleotide binding to the CRISPR effector protein is less than 8 pM (pM=picomolar). The equilibrium dissociation constant (K_{d}) can be less than 7 pM, 5 pM, 4 pM, 3 pM, 2 pM, 1 pM, 9 fM (fM=femtomolar), 8 fM, 7 fM, 6 fM, 5 fM, 4 fM, 3 fM, 2 fM, 1 fM, 9 aM (aM=attomolar), 8 aM, 7 aM, 6 aM, 5 aM, 4 aM, 3 aM, 2 aM, or 1 aM. The equilibrium dissociation constant (K_{d}) can be from about 1 pM to 8 pM, from about 1 fM to about 10 fM, or from about 1 aM to about 10 aM. The CRISPR effector protein can be covalently attached to the CRISPR polynucleotide. In some cases, the CRISPR effector protein is not covalently attached to the CRISPR polynucleotide.

### IV. Methods of Using a Stabilized CRISPR Complex

Provided herein are methods of using stabilized (e.g., locked) CRISPR complexes described herein.

### A. Administration to a Cell

An aspect of the present disclosure encompasses a method for administering a stabilized (e.g., conjugated or “locked”) CRISPR complex to a cell. A stabilized CRISPR complex can comprise a CRISPR polynucleotide comprising an unnatural nucleotide to crosslink to a CRISPR effector protein and an activity modulating sequence (e.g. CRISPR ON, CRISPR OFF, or CRISPR ON/OFF, described herein). The method can comprise contacting the cell with a solution of stabilized (e.g., conjugated or “locked”) CRISPR complex. Alternatively, or in combination, the method can comprise contacting a cell with a vector comprising CRISPR effector protein encoding regions and/or CRISPR polynucleotide encoding regions. Alternatively, or in combination non-viral mediated techniques can be used to introduce a CRISPR polynucleotide into a cell. Non-viral mediated techniques can include electroporation, calcium phosphate mediated transfer, nucleofection, sonoporation, heat shock, magnetofection, liposome mediated transfer, microinjection, microprojectile mediated transfer, nanoparticles, cationic polymer mediated transfer (e.g., DEAE-dextran, polyethylenimine, PEG, DMSO, etc.) or cell fusion.

Viral and non-viral mediated techniques can be used to introduce a CRISPR polynucleotide into a cell. The non-viral mediated techniques can be electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion.

The polynucleotide modified with at least one unnatural nucleotide for crosslinking comprising a CRISPR ON polynucleotide sequence, CRISPR OFF polynucleotide sequence, or CRISPR ON/OFF polynucleotide sequence, described herein and related vectors can be delivered to a cell naked (i.e., free from agents which promote transfection). The naked CRISPR polynucleotides can be delivered to the cell using routes of administration known in the art and described herein.

In some cases, the tunable modulation of the editing of a target gene in a target DNA in a host cell comprises the steps: (i) using viral or non-viral delivery methods or a combination thereof, described herein known in the art, to introduce into the host cell: (a) a CRISPR polynucleotide comprising an unnatural nucleotide to crosslink the CRISPR polynucleotide to a CRISPR effector protein, and a first and second cleavage elements, where the cleavage elements are susceptible to cleavage and where the nucleotide sequence of the guide sequence is fully or partially complementary to a target nucleic acid sequence, wherein the first cleavage element is position between a polynucleotide leader sequence and a 5′ end of a guide sequence; and (b) a CRISPR effector protein (e.g., CRISPR enzyme, e.g., Cas9) with catalytic activity such that the CRISPR polynucleotide and the CRISPR enzyme form a CRISPR complex; and (ii) through exposure to UV light, inducing cleavage of the first sequence element in the polynucleotide, thereby releasing the polynucleotide leader sequence and activating higher target specific cleavage of the target gene by the CRISPR complex. Subsequently, the method can comprise (iii) inducing cleavage of the second sequence element, which can be located in scaffold sequence of the CRISPR polynucleotide, at a desired time through pulsed exposure to UV light, thereby cleaving the CRISPR polynucleotide and deactivating or lowering the target specific cleavage of the target gene by the CRISPR complex.

The cell can be ectodermal (e.g., neurons and fibroblasts), mesodermal (e.g., cardiac muscle cells), endodermal (e.g., pancreatic cells), epithelial (e.g., lung and nasal passageways), neutrophils, eosinophils, basophils, lymphocytes, osteoclasts, endothelial cells, hematopoietic, red blood cells, etc. The cell can be derived from specific cell lines such as CHO cells (e.g., CHOKl); HEK293 cells; Caco2 cells; U2-OS cells; NIH 3T3 cells; NSO cells; SP2 cells; DG44 cells; K-562 cells, U-937 cells; MC5 cells; IMR90 cells; Jurkat cells; HepG2 cells; HeLa cells; HT-1080 cells; HCT-116 cells; Hu-h7 cells; Huvec cells; and Molt 4 cells. Examples of other cells applicable to the scope of the present disclosure can include stem cells, embryonic stem cells (ESCs) and induced pluripotent stem cells (iPSCs)., MSC-1, K562, etc.

In some cases, masks can be created to go over a cell culture. Mask can be created using a variety of techniques (laser cutting, 3D printing, photolithography, etc.) Masks can be designed to let light penetrate in a defined region. When used in conjunction with a CRISPR OFF complex comprising a photocleavable linker, editing in areas where the light (e.g., UV light or visible light) penetrates can be decreased, and editing in areas without exposure to light can be maintained. When used in conjunction with a CRISPR ON complex, editing in areas where the light (e.g., UV light) penetrates can be initiated.

In some cases, a CRISPR OFF complex activity can be time-dependent (e.g., as can be seen in Example 6, FIGS. 30-32). Cells can be exposed to a cleavage activator, such as UV light or visible light, at a time point prior to complete editing, resulting in a heterozygous clone. Alternatively, such a method can be used to target a diseased allele of a patient-derived cell line.

The stabilized (e.g., conjugated or “locked”) CRISPR complex, with a dissociation constant near zero, can have an increased efficacy over current complexes in which the CRISPR polynucleotide can dissociate from the CRISPR effector protein prior to or during administration to a cell.

### 1. Multiple CRISPR Complexes

In some cases, a system comprises one or more CRISPR complexes provided herein. A first and second (or more) CRISPR complexes can be used in an in vitro or in vivo method. The CRISPR effector proteins in the first and second CRISPR complexes can be the same or different. In one example, an in vitro or in vivo system can comprise a plurality of CRISPR polynucleotides with different guide sequences and the same CRISPR effector protein (e.g, Cas9). In another example, an in vitro or in vivo system can comprise a CRISPR polynucleotide and a plurality of different CRISPR effector proteins (e.g., a mix of catalytically active and catalytically inactive CRISPR effector proteins).

Alternatively, or in combination, a host cell can comprise two or more (e.g., 2, 3, 4, 5, 6, 7, 8, 9, or 10) different locked CRISPR complexes, wherein the nucleotide sequences of the guide sequence of the different CRISPR complexes are independently fully or partially complementary to regions of two or more different target nucleic acids (e.g., DNA). The different CRISPR complexes can have different relative positions of one or more cleavage elements, or the same relative positions of the one or more cleavage elements.

### B. Cleaving Target Nucleic Acid

An aspect of the present disclosure encompasses a method for cleaving a target nucleic acid. The method can comprise contacting a nucleic acid sequence with a stabilized (e.g., conjugated or “locked”) CRISPR complex. A stabilized CRISPR complex can comprise a CRISPR polynucleotide comprising an unnatural nucleotide to crosslink to a CRISPR effector protein and an activity modulating sequence (e.g. CRISPR ON, CRISPR OFF, or CRISPR ON/OFF, described herein). A locked CRISPR complex, e.g., with crosslinking sites chosen so as to not interfere with the nuclease activity of the Cas nuclease or the binding efficiency of the crRNA region, can have an enhanced efficacy in the cleavage of a target nucleic acid. The polynucleotide can comprise a “protospacer” and a “protospacer adjacent motif (PAM), and both domains can be needed for a CRISPR effector protein mediated activity (e.g., cleavage).

Each catalytic domain of the CRISPR effector protein can be active alternatively or in combination. Efficiency of each catalytic domain can be from 50% to 60%, 60% to 70%, 70% to 80%, 80% to 90%, or 90% to 99.9%.

After cleavage, DNA repair can occur by non-homologous end joining (NHEJ), microhomology mediated end joining (MMEJ, alternative nonhomologous end joining) or homology directed repair (HDR). A DNA template can be provided for HDR.

In some examples, the cleavage can lead to insertion and/or deletion (“indel”) mutations or a frameshift by a nonhomologous end joining (NHEJ) process, leading to a target gene-specific knockout (KO). In some cases, CRISPR/Cas complex can be directed to the target genomic region by the specific gRNA (e.g., sgRNA) along with a co-administered, donor polynucleotide (single- or double-stranded). Following the cleavage of the target region, a homology-directed repair (HDR) process can use one or more of the donor polynucleotide as one or more templates for (a) repair of the cleaved target nucleotide sequence and (b) a transfer of genetic information from the donor polynucleotide to the target DNA. Depending on the nature of the genetic information, the HDR process can yield a target gene-specific KO or knock-in (KI). Examples of applications of the HDR-mediated gene KI include the addition (insert or replace) of nucleic acid material encoding for a protein, mRNA, small interfering RNA (siRNA), tag (e.g., 6×His), a reporter protein (e.g., a green fluorescent protein (GFP)), and a regulatory sequence to a gene (e.g., a promotor, polyadenylation signal).

For the HDR process, the donor polynucleotide can contain the desired edit, e.g., gene edit (sequence) to be copied, as well as additional nucleotide sequences on both ends (homology arms) that are homologous immediately upstream and downstream of the cleaved target site. In some cases, the efficiency of the HDR process can depend on the size of the gene edit and/or the size of the homology arms.

One or more CRISPR complexes can be provided to target one or more cleavage sites. For example, two CRISPR complexes can be provided to target two cleavage sites, ten CRISPR complexes can be provided to target ten cleavage sites, twenty CRISPR complexes can be provided to target twenty cleavage sites, etc. The number of different CRISPR polynucleotides (e.g., sgRNAs) that can be provided to a cell can be about 1, 2, 3, 4, 5, 6, 7, 8, 9, 10, 11, 12, 13, 14, 15, 25, 50, 100, or 1000, or 1 to 3, 1 to 5, 1 to 10, 10 to 50, or 50 to 100.

CRISPR complexes described herein can induce one or more edits or mutations in a cell. The one or more edits or mutations can include the introduction, deletion, or substitution of one or more nucleotides at each target sequence of cells via CRISPR polynucleotides (e.g., the guides RNAs or sgRNAs). The one or more edits or mutations can be introduction, deletion, or substitution of about 1 to about 75 nucleotides at each target sequence of said cells. The one or more edits or mutations can be the introduction, deletion, or substitution of 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell. Target sequences can be genes and can include BUB1B, CAMK1, PRKAG3, STK3, CAMK1, Chr8q23, CEL, IRAK4, DNMT1, EMX1, FANCF, GRK1, PRGN, AAVS1, BUB1B, CXCR4, FAM163A, GAA, CRK1, IRAK4, MAPRE1, MIP, OMP, OPN1SW, PRKAG3, STK3, and VEGFA (as can be seen in Examples 7, 8, 11, 12).

Multiple target sites can be targeted by sets of CRISPR complexes attached to different sgRNAs. Each gRNA in the set can be hybridizable to a region that is at most 170 bases apart from the hybridizable region of at least one other guide RNA from the set of guide RNAs. Each gRNA in the set of gRNAs that target the genomic region of interest can be hybridizable to a region that is about 10 to 200 bases (nucleotides) apart from the hybridizable region of the at least one other gRNA from the set of gRNAs. Each gRNA in the set of gRNAs can be hybridizable to a region that is at least 10, 15, 20, 25, 30, 25, 40, 45, 50, 60, 70, 80, 90, 100, 120, 140, 160, 180, 200 or more bases apart from the hybridizable region of the at least one other gRNA from the set of gRNAs. Each gRNA in the set of gRNAs can be hybridizable to a region that is at most 200, 180, 160, 140, 120, 100, 90, 80, 70, 60, 50, 45, 40, 35, 30, 25, 20, 15, 10, or less bases apart from the hybridizable region of the at least one other gRNA from the set of gRNAs. In an example, a minimum distance between a hybridizable region of a gRNA in the set of gRNAs is at least 30 bases apart from the hybridizable region of at least one other gRNA from the set of gRNAs. In another example, a maximum distance between a hybridizable region of a gRNA in the set of gRNAs is at most 150 bases apart from the hybridizable region of at least one other gRNA from the set of gRNAs.

In some cases, the CRISPR/Cas activity can be useful in any in vitro or in vivo application in which it is desirable to modify DNA in a site-specific (targeted) way, for example gene knock-out (KO), gene knock-in (KI), gene editing, gene tagging, etc., as used in, for example, gene therapy. Examples of gene therapy include treating a disease or as an antiviral, antipathogenic, or anticancer therapeutic; the production of genetically modified organisms in agriculture; the large scale production of proteins by cells for therapeutic, diagnostic, or research purposes; the induction of induced pluripotent stem cells (iPS cells or iPSCs); and the targeting of genes of pathogens for deletion or replacement.

### C. Gene Regulation

An aspect of the present disclosure encompasses a method for regulating gene expression, or the transcription of a gene to mRNA, known as a knockdown method by targeting, to a gene, functional domains such as repressor domains and activator domains. A catalytically dead Cas nuclease linked to a transcription repressor (e.g., KRAB, DMT3A, and/or LSD1) and bound CRISPR polynucleotide (e.g., sgRNA) can bind to a complementary DNA region in a gene and block transcription. An embodiment of the method encompasses rendering a Cas nuclease catalytically dead upon photoinitiated crosslinking of the sgRNA to the Cas nuclease while maintaining the complementary binding activity of the sgRNA in the locked CRISPR complex to a target DNA sequence. In some cases, a catalytically dead CRISPR effector protein (e.g., Cas) can be fused to one or more transcriptional activators (e.g., VP64, p65, and/or RTa19), and a stabilized (e.g., conjugated or “locked”) complex can be formed with a CRISPR polynucleotide (e.g., sgRNA). The stabilized (e.g., conjugated or “locked”) complex can be delivered to a gene in a cell to upregulate transcription of the gene.

In some cases, the functional domain can be linked to a dead CRISPR effector protein (e.g., dead-CRISPR effector protein within a cell, a CRISPR complex can be formed.). The CRISPR ON polynucleotide comprising an unnatural nucleotide to crosslink with the CRISPR effector protein, can further comprises a polynucleotide leader sequence separated from a guide sequence by a photocleavable element. The cell can be exposed to UV radiation, resulting in cleavage of the cleavage element and release of the polynucleotide leader sequence. The CRISPR complex can then cleave target sequence. In some cases, a donor nucleic acid is also introduced into the cell, which can be used in homologous recombination at the cleavage site to introduce an edit to the nucleic acid.

The nucleic acid editing can target an endogenous regulatory control element (e.g., enhancer or silencer). The nucleic acid editing can target a promoter or promoter-proximal elements. These control elements can be located upstream or downstream of the transcriptional start site (TSS), starting from 200 bp from the TSS to 100 kb away. Targeting of known control elements can be used to activate or repress a gene of interest. A single control element can influence the transcription of multiple target genes. Targeting of a single control element can therefore be used to control the transcription of multiple genes simultaneously.

The one or more functional domains can be a nuclear localization sequence (NLS) or a nuclear export signal (NES).

The one or more functional domains can be a transcriptional activation domain. The transcriptional activation domain can be VP64, p65, MyoD1, HSF1, RTA, SET7/9, or a histone acetyltransferase. The CRISPR effector protein can be a dead Cas protein-fused with a domain with transcriptional activator or repressor activity. The dead Cas protein fused with a domain with transcriptional activator or repressor activity can be used to study the epistatic interactions between a given pair of genes in a specific tissue.

The one or more functional domains can have one or more activities comprising methylase activity, demethylase activity, transcription activation activity, transcription repression activity, transcription release factor activity, histone modification activity, RNA cleavage activity, DNA cleavage activity, DNA integration activity or nucleic acid binding activity.

The one or more functional domains can be a transposase domain, integrase domain, recombinase domain, resolvase domain, invertase domain, protease domain, DNA methyltransferase domain, DNA hydroxylmethylase domain, DNA demethylase domain, histone acetylase domain, histone deacetylases domain, nuclease domain, repressor domain, activator domain, nuclear-localization signal domains, transcription-regulatory protein (or transcription complex recruiting) domain, cellular uptake activity associated domain, nucleic acid binding domain, antibody presentation domain, histone modifying enzymes, recruiter of histone modifying enzymes; inhibitor of histone modifying enzymes, histone methyltransferase, histone demethylase, histone kinase, histone phosphatase, histone ribosylase, histone deribosylase, histone ubiquitinase, histone deubiquitinase, histone biotinase, or histone tail protease.

In some cases, the functional domain can be linked to a dead CRISPR effector protein (e.g., dead-Cas protein). The functional domain linked to the dead CRISPR effector protein (e.g., dead-Cas protein) can used to bind to and/or activate a promoter or enhancer. One or more CRISPR polynucleotides comprising a guide sequence that can anneal to the promoter or enhancer can also be provided to direct the binding of a CRISPR complex comprising a CRISPR effector protein (e.g., dead-Cas) to the promoter or enhancer. A CRISPR ON/OFF polynucleotide can be covalently crosslinked to a CRISPR effector protein, which can be a catalytically dead Cas9. The catalytically dead Cas9 can be fused to a transcription activation domain (e.g., VP64). The fusion, e.g., Cas9-VP64 fusion, and can be used to tunably modulate the expression of a target gene or a chromatin region. For example, the polynucleotide leader sequence of the CRISPR ON/OFF polynucleotide can prevent efficient localization of the CRISPR complex to target gene via the guide sequence. Cleavage of the polynucleotide leader sequence can result in efficient targeting of the CRISPR complex to the target sequence via the guide sequence, which can result in transcriptional activation. Subsequently, a second cleavage agent can be exposed to the CRISPR polynucleotide that results in cleavage of the CRISPR polynucleotide and reduces or inhibits the ability of the CRISPR complex (or CRISPR effector protein, if the cleaved CRISPR polynucleotide has dissociated from the CRISPR effector protein) to activate transcription of the gene.

Targeting of regions with either an activation or repression system described herein can be followed by readout of transcription of either a) a set of putative targets (e.g., a set of genes located in closest proximity to the control element) or b) whole-transcriptome readout by e.g. RNAseq or microarray.

In another example, CRISPR complexes provided herein can be used to study the epistatic interactions of two or more target genes in the host cell. A method can comprise of the steps: (i) using viral or non-viral delivery methods or a combination thereof, introducing into the host cell: (a) a CRISPR polynucleotide comprising first and second cleavage elements, where the cleavage elements are susceptible to cleavage and where the nucleotide sequence of the guide sequence is fully or partially complementary to a first target nucleic acid sequence; (b) a CRISPR effector protein (e.g., CRISPR) enzyme with catalytic activity, such that the CRISPR polynucleotide (e.g., sgRNA) and the CRISPR enzyme form a CRISPR complex; and (ii) at the desired time, inducing the cleavage of the first cleavage element in the CRISPR polynucleotide and activating higher target specific cleavage of the target gene by the CRISPR complex and then (iii) inducing cleavage of the second cleavage element at a desired time, thereby deactivating or lowering the target specific cleavage of the target gene by the CRISPR complex.

The method can further comprise (i) using viral or non-viral delivery methods or a combination thereof to introduce into the host cell: (a) a second CRISPR polynucleotide comprising of the first and second cleavage elements, where the cleavage elements are susceptible to cleavage and where the nucleotide sequence of the guide sequence is fully or partially complementary to a region of a second target sequence (e.g., in a target gene); (b) a CRISPR enzyme with catalytic activity, such that the second CRISPR polynucleotide (e.g., sgRNA) and the CRISPR enzyme form a second CRISPR complex; and (ii) at the desired time, inducing the cleavage of the first cleavage element in the second CRISPR polynucleotide and activating higher target specific cleavage of the target gene by the CRISPR complex and then (iii) inducing cleavage of the second cleavage element at a desired time, thereby deactivating or lowering the target specific cleavage of the target gene by the second CRISPR complex.

Furthermore, the cleavage of the first cleavage element in the first and second CRISPR complex can be under control of a tissue specific promoter, e.g., a muscle specific promoter. For example, expression of genetically engineered endoribonuclease Cas6a/Csy4 in the cell can be placed under the control of a tissue-specific promoter (e.g., muscle) promoter that can be activated at given times to induce cleavage of the first cleavage element. The second cleavage element in the first and second CRISPR complex can be inducibly cleaved at a desired time by exposure to a given sequence-specific small molecule. The CRISPR enzyme can be a dCas9-fused with a domain with transcriptional activator or repressor activity and can be used to study the epistatic interactions between a given pair of genes in a specific tissue.

In another example, CRISPR complexes described herein can be used to induce orthogonal transcription of two or more target genes in one or more target DNAs in a host cell. The term “orthogonal” can mean independent, i.e., the two or more target genes can be independently regulated or independently transcribed. The method can comprise the steps of using viral or non-viral delivery methods or a combination thereof for introducing into the host cell: (a) two or more different inducible CRISPR polynucleotides comprising of first and second cleavage elements, where the first and second sequence elements are susceptible to cleavage and where the nucleotide sequence of the guide sequence is fully or partially complementary to one or more target DNAs in the vicinity of the two or more different target genes; (b) a catalytically-inactive CRISPR enzyme linked to a transcriptional activator domain, such that the different inducible CRISPR polynucleotides and the CRISPR enzyme form different CRISPR complexes, wherein the CRISPR complexes comprise one or more effector domains; and (ii) at the desired times, inducing the cleavage of the first cleavage element in the first and second polynucleotide and thus coordinating the expression of the target genes. The target DNAs can be adjacent regions within a single gene or control element.

### D. Pharmaceutical Formulation

The CRISPR polynucleotides and CRISPR complexes described herein can be used in vitro or in vivo to cause a change in a cell or an organism. The CRISPR polynucleotide and CRISPR effector protein can be introduced as a complex or they can form a complex within the cell. The CRISPR polynucleotide and/or CRISPR effector protein can be passively introduced to a cell or introduced through a vehicle. The CRISPR polynucleotide and the CRISPR effector protein can be present in a buffer at the time of introduction.

An aspect of the present disclosure encompasses a method for manufacturing stabilized (e.g., conjugated or “locked”) CRISPR complexes for use in pharmaceutical formulations. CRISPR complexes can be prepared for delivery by, for example, liposomes and nanoparticle delivery. Alternatively, or in combination, vectors coding for CRISPR effector protein and/or CRISPR polynucleotide can be delivered to a patient by, for example, microinjection or other mechanical, physical, or viral methods. The CRISPR complexes and related vector constructs can be used in combination with one or more therapeutic, prophylactic, diagnostic, or imaging agents.

Pharmaceutical formulations can comprise one or more excipients to increase stability, enhance transfection to a cell, control release (such as from a carrier, e.g., nanoparticle), alter biodistribution, or alter translation of a vector encoding the CRISPR complex. Pharmaceutical formulations can comprise mixtures of the crosslinked CRISPR complex, water, co-solvents, buffer agents, and pH-adjusting agents. Co-solvent excipients can comprise oil, surfactants, emulsifiers, stabilizers, chelators, and preservatives. Stabilizers can include sugars and amino acids. Sugars can include sucrose and lactose. Amino acids can include glycine and monosodium glutamate. Preservatives can include phenol, phenoxyethanol and thimersosal.

The locked CRISPR complex can be formulated using one or more excipients to: (1) increase stability; (2) increase cell transfection; (3) permit the sustained or delayed release (e.g., from a depot formulation of the polynucleotide); (4) alter the biodistribution (e.g., target the polynucleotide, primary construct, or mRNA to specific tissues or cell types); (5) increase the translation of encoded protein in vivo; and/or (6) alter the release profile of encoded protein in vivo.

The excipients can be solvents, dispersion media, diluents, or other liquid vehicles, dispersion or suspension aids, surface active agents, isotonic agents, thickening or emulsifying agents, preservatives, and/or emulsifiers, preservatives, buffering agents, lubricating agents, and/or oils. The excipients can be lipidoids, liposomes, lipid nanoparticles, polymers, lipoplexes, core-shell nanoparticles, peptides, proteins, cells transfected with polynucleotide, primary construct, or Cas nuclease mRNA (e.g., for transplantation into a subject), hyaluronidase, nanoparticle mimics and combinations thereof.

Relative amounts CRISPR polynucleotide, CRISPR effector protein, or nucleic acid encoding either, and the pharmaceutically acceptable excipient, and/or any additional ingredients in a pharmaceutical composition can vary, depending upon the identity, size, and/or condition of the subject treated and further depending upon the route by which the composition is to be administered. The composition can comprise between 0.1% and 100%, e.g., between 0.5 and 50%, between 1-30%, between 5-80%, at least 80% (w/w) CRISPR polynucleotide, CRISPR effector protein, or nucleic acid encoding either.

The CRISPR polynucleotide sequence comprising at least one unnatural nucleotide for crosslinking and an activity modulating element such as a CRISPR ON polynucleotide, CRISPR OFF polynucleotide, or CRISPR ON/OFF polynucleotide, described herein can be formulated in pharmaceutical compositions comprising one or more pharmaceutically acceptable excipients. The pharmaceutical compositions can comprise one or more additional active substances, e.g., therapeutically and/or prophylactically active substances. General considerations in the formulation and/or manufacture of pharmaceutical compositions can be found, for example, in Remington: The Science and Practice of Pharmacy 2 ed., Lippincott Williams & Wilkins, 2005 (incorporated herein by reference in its entirety).

The synthesis of lipidoids has been extensively described and formulations containing these compounds are particularly suited for delivery of the modified CRISPR ON polynucleotide, CRISPR OFF polynucleotide, or CRISPR ON/OFF polynucleotide, described herein and primary constructs (see Mahon et al., Bioconjug Chem. 2010 21: 1448-1454; Schroeder et al., J Intern Med. 2010 267:9-21; Akinc et al, Nat Biotechnol. 2008 26:561-569; Love et al, Proc Natl Acad Sci USA. 2010 107: 1864-1869; Siegwart et al., Proc Natl Acad Sci USA. 2011 108: 12996-3001; all of which are incorporated herein in their entireties). Different ratios of lipidoids and other components including, but not limited to, disteroylphosphatidyl choline, cholesterol and PEG-DMG, can be used to optimize the formulation of the polynucleotide, primary construct, or Cas nuclease mRNA for delivery to different cell types including, but not limited to, hepatocytes, myeloid cells, muscle cells, etc.

The locked CRISPR complex can be formulated using one or more liposomes, lipoplexes, or lipid nanoparticles (LNP). The pharmaceutical compositions can include liposomes. The pharmaceutical compositions described herein can include liposomes such as those formed from the synthesis of stabilized plasmid-lipid particles (SPLP) or stabilized nucleic acid lipid particle (SNALP) that have been previously described and shown to be suitable for polynucleotide delivery in vitro and in vivo (see Wheeler et al. Gene Therapy. 1999 6:271-281; Zhang et al. Gene Therapy. 1999 6: 1438-1447; Jeffs et al. Pharm Res. 2005 22:362-372; Morrissey et al, Nat Biotechnol. 2005 2: 1002-1007). The CRISPR polynucleotides can be formulated in a lipid vesicle which can have crosslinks between functionalized lipid bilayers.

The locked CRISPR complex can be formulated in a lipid-polycation complex. The formation of the lipid-polycation complex can be accomplished by methods known in the art and/or as described in U.S. Pub. No. 20120178702, herein incorporated by reference in its entirety. The pharmaceutical composition can include at least one of the PEGylated lipids described in International Publication No. 2012099755, herein incorporated by reference.

The LNP formulation can be formulated by the methods described in International Publication Nos. WO2011 127255 or WO2008 103276, each of which is herein incorporated by reference in their entirety. The CRISPR polynucleotide can be encapsulated in LNP formulations as described in WO2011 127255 and/or WO2008103276; each of which is herein incorporated by reference in their entirety.

The locked CRISPR complex can be formulated as a solid lipid nanoparticle. A solid lipid nanoparticle (SLN) can be spherical with an average diameter between 10 to 1000 nm. SLN can possess a solid lipid core matrix that can solubilize lipophilic molecules and can be stabilized with surfactants and/or emulsifiers. The lipid nanoparticle can be a self-assembly lipid-polymer nanoparticle (see Zhang et al, ACS Nano, 2008, 2 (8), pp 1696-1702; herein incorporated by reference in its entirety).

The locked CRISPR complex, primary constructs, or the Cas nuclease mRNA can be encapsulated into a lipid nanoparticle or a rapidly eliminating lipid nanoparticle and the lipid nanoparticles or a rapidly eliminating lipid nanoparticle can then be encapsulated into a polymer, hydrogel and/or surgical sealant described herein and/or known in the art.

The locked CRISPR complex formulation for controlled release and/or targeted delivery can also include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose,

The controlled release and/or targeted delivery formulation can comprise at least one degradable polyester which can contain polycationic side chains. The degradable polyester can be poly(serine ester), poly(L-lactide-co-L-lysine), poly(4-hydroxy-L-proline ester), and combinations thereof. The degradable polyesters can include a PEG conjugation to form a PEGylated polymer.

The locked CRISPR complex can be encapsulated in a therapeutic nanoparticle. The therapeutic nanoparticle can be formulated for sustained release. The period of time can include hours, days, weeks, months and years. As a non-limiting example, the sustained release nanoparticle can comprise a polymer and a therapeutic agent, e.g., CRISPR polynucleotides described herein (see International Pub No. 2010075072 and US Pub No. US20100216804 and US20110217377, each of which is herein incorporated by reference in their entirety. The therapeutic nanoparticles can be formulated to be target specific. The therapeutic nanoparticles can include a corticosteroid (see International Pub. No. WO2011084518).

The locked CRISPR complex can be encapsulated in, linked to and/or associated with synthetic nanocarriers. The synthetic nanocarriers can be formulated by the methods described in International Pub Nos. WO2010005740, WO2010030763. The synthetic nanocarriers can contain reactive groups to release the CRISPR polynucleotides, described herein (see International Pub. No. WO20120952552 and US Pub No. US20120171229, each of which is herein incorporated by reference in their entirety).

The synthetic nanocarriers can be formulated for targeted release. The synthetic nanocarrier can be formulated to release the CRISPR complex at a specified pH and/or after a desired time interval. The synthetic nanoparticle can be formulated to release the polynucleotides, primary constructs and/or Cas nuclease mRNA after 24 hours and/or at a pH of 4.5 (see International Pub. Nos. WO2010138193 and WO2010138194 and US Pub Nos. US20110020388 and US20110027217, each of which is herein incorporated by reference in their entireties).

The synthetic nanocarriers can be formulated for controlled and/or sustained release of the locked CRISPR complex described herein. The synthetic nanocarriers for sustained release can be formulated, e.g., as described herein and/or as described in International Pub No. WO2010138192 and US Pub No. 20100303850, each of which is herein incorporated by reference in their entireties.

The locked CRISPR complex can be formulated with or in a polymeric compound. The polymer can include at least one polymer polyethenes, polyethylene glycol (PEG), poly(1-lysine) (PLL), PEG grafted to PLL, cationic lipopolymer, biodegradable cationic lipopolymer, polyethyleneimine (PEI), conjugated branched poly(alkylene imines), a polyamine derivative, a modified poloxamer, a biodegradable polymer, biodegradable block copolymer, biodegradable random copolymer, biodegradable polyester copolymer, biodegradable polyester block copolymer, biodegradable polyester block random copolymer, linear biodegradable copolymer, poly[a-(4-aminobutyl)-L-glycolic acid) (PAGA), biodegradable conjugated cationic multi-block copolymers, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester), acrylic polymers, amine-containing polymers or combinations thereof.

The locked CRISPR complex described herein can be conjugated with another compound. The CRISPR polynucleotide can also be formulated as a nanoparticle using a combination of polymers, lipids, and/or other biodegradable agents, e.g., calcium phosphate. Components can be combined in a core-shell, hybrid, and/or layer-by-layer architecture, to allow for fine-tuning of the nanoparticle so the delivery of the CRISPR polynucleotide can be enhanced (Wang et al, Nat Mater. 2006 5:791-796; Fuller et al, Biomaterials. 2008 29: 1526-1532; DeKoker et al, Adv Drug Deliv Rev. 2011 63:748-761; Endres et al., Biomaterials. 2011 32:7721-7731; Su et al., Mol Pharm. 2011 Jun. 6; 8(3):774-87; herein incorporated by reference in its entirety).

The locked CRISPR complex can be formulated with peptides and/or proteins in order to increase transfection of cells by the locked CRISPR complex. The peptides can be cell penetrating peptides and proteins and peptides that enable intracellular delivery can be used to deliver pharmaceutical formulations.

The locked CRISPR complex can be transfected ex vivo into cells, and subsequently transplanted into a subject. Examples of such vectors include primary nucleic acid constructs or synthetic sequences encoding CRISPR effector proteins or related polypeptides. The pharmaceutical compositions can include red blood cells to deliver modified RNA to liver and myeloid cells, virosomes to deliver modified RNA in virus-like particles (VLPs), and electroporated cells e.g., from MAXCYTE® (Gaithersburg, MD) and from ERYTECH® (Lyon, France) to deliver modified RNA.

Cell-based formulations of the locked CRISPR complex disclosed herein or related vector constructs can be used to ensure cell transfection (e.g., in the cellular carrier), alter the biodistribution of the locked CRISPR complex (e.g., by targeting the cell carrier to specific tissues or cell types), and/or increase the translation of encoded protein.

The compositions can also be formulated for direct delivery to an organ or tissue by, e.g., direct soaking or bathing, via a catheter, by gels, powder, ointments, creams, gels, lotions, and/or drops, by using substrates such as fabric or biodegradable materials coated or impregnated with the compositions, and the like.

An aspect of the present disclosure encompasses a method for administering a pharmaceutical formulation to a patient. Unbound RNA can elicit an immune response by interferon gamma. A locked CRISPR complex can greatly lessen the likelihood of an unbound sgRNA in a pharmaceutical composition. Linked CRISPR complexes and related sequences/polypeptides can be administered by any route which results in a therapeutically effective outcome. These include enteral, gastroenteral, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection, (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops. Compositions can be administered in a way which allows them to cross the blood-brain barrier, vascular barrier, or other epithelial barrier.

The pharmaceutical formulation can be administered to a subject in need thereof 4 times a day, 3 times a day, 2 times a day, daily, three times a week, two times a week, weekly, four times a month, 3 times a month, 2 times a month, monthly, 4 times a year, 3 times a year, 2 times a year, or annually. The administration can be for over a period of time, and the period of time can be at least or up to 1 week, at least or up to 1 month, at least or up to 1 year, at least or up to 10 years, or a lifetime of the subject.

### E. Conditions

An aspect of the present disclosure encompasses the treatment of a disease condition with CRISPR complexes. Disease conditions can include cancer, neurological conditions, autoimmune disorders, etc. Treatment of a disease condition can comprise treatment of a diseased tissue. Treatment can comprise the treatment of cells with the CRISPR complex followed by injecting, grafting, or implanting the cells into a human patient.

The CRISPR ON polynucleotide, CRISPR OFF polynucleotide, or CRISPR ON/OFF polynucleotide, described herein can be used in a number of different scenarios in which delivery of a substance (the “payload”) to a biological target is desired, for example delivery of detectable substances for detection of the target, or delivery of a therapeutic agent. The CRISPR polynucleotides and related vector constructs can be used in combination with one or more other therapeutic, prophylactic, diagnostic, or imaging agents.

The CRISPR ON polynucleotide, CRISPR OFF polynucleotide, or CRISPR ON/OFF polynucleotide, described herein and other primary constructs can be designed to include both a linker and a payload in any useful orientation. For example, a linker having two ends can be used to attach one end to the payload and the other end to the nucleobase, such as at the C-7 or C-8 positions of the deaza-adenosine or deaza-guanosine or to the N-3 or C-5 positions of cytosine or uracil. The payload can be a therapeutic agent such as a cytotoxin, radioactive ion, chemotherapeutic, or other therapeutic agent.

The CRISPR ON polynucleotide, CRISPR OFF polynucleotide, or CRISPR ON/OFF polynucleotide, described herein can be used to alter the phenotype of cells. The CRISPR polynucleotide or CRISPR effector protein encoding sequence can be used in therapeutics and/or clinical and research settings. The CRISPR ON polynucleotide, CRISPR OFF polynucleotide, or CRISPR ON/OFF polynucleotide, described herein and related vector constructs and the proteins translated from them described herein can be used as therapeutic or prophylactic agents. For example, a CRISPR polynucleotide or Cas nuclease mRNA described herein (e.g. a modified mRNA encoding a CRISPR-related polypeptide or effector protein) can be administered to a subject, and translated in vivo to direct the expression of a therapeutically relevant or prophylactic polypeptide in the subject.

The ability of a guide sequence (within a nucleic acid-targeting guide RNA or sgRNA) to direct sequence-specific binding of a nucleic acid-targeting complex to a target nucleic acid sequence can be assessed by any suitable assay. For example, the components of a nucleic acid-targeting CRISPR system sufficient to form a nucleic acid-targeting complex, including the guide sequence to be tested, can be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence. Cleavage of a target nucleic acid sequence can be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions.

Compositions provided herein can be used for treatment of any of a variety of diseases, disorders, and/or conditions, e.g., one or more of the following: autoimmune disorders (e.g. diabetes, lupus, multiple sclerosis, psoriasis, rheumatoid arthritis); inflammatory disorders (e.g. arthritis, pelvic inflammatory disease); infectious diseases (e.g. viral infections (e.g., HIV, HCV, RSV), bacterial infections, fungal infections, sepsis); neurological disorders (e.g. Alzheimer's disease, Huntington's disease; autism; Duchenne muscular dystrophy); cardiovascular disorders (e.g. atherosclerosis, hypercholesterolemia, thrombosis, clotting disorders, angiogenic disorders such as macular degeneration); proliferative disorders (e.g. cancer, benign neoplasms); respiratory disorders (e.g. chronic obstructive pulmonary disease); digestive disorders (e.g. inflammatory bowel disease, ulcers); musculoskeletal disorders (e.g. fibromyalgia, arthritis); endocrine, metabolic, and nutritional disorders (e.g. diabetes, osteoporosis); urological disorders (e.g. renal disease); psychological disorders (e.g. depression, schizophrenia); skin disorders (e.g. wounds, eczema); blood and lymphatic disorders (e.g. anemia, hemophilia); etc.

In one aspect, the disease condition can be cardiovascular disease, diabetes, lung diseases, chronic obstructive pulmonary disease (COPD), asthma, idiopathic pulmonary fibrosis, chronic bronchitis, cystic fibrosis, coronary heart disease, cerebrovascular diseases, etc. In one aspect, the disease condition can be Alzheimer's disease, Amyotrophic lateral sclerosis (ALS), arteriovenous malformation, multiple sclerosis (MS), or Parkinson's disease. In another aspect, the disease condition can be psoriasis, Graves' disease, Guillain-Barre syndrome, hashimoto's thyroiditis, vasculitis, myasthenia gravis, chronic inflammatory demyelinating polyneuropathy, Type 1 diabetes mellitus, multiple sclerosis, systemic lupus, rheumatoid arthritis, etc.

In one aspect, the disease condition can be biliary tract cancer (e.g., adenocarcinoma), lung cancer (e.g., large cell carcinoma, non small cell carcinoma, squamous cell carcinoma, neoplasia, etc.), colorectal cancer, prostate cancer, endometrial cancer, ovarian cancer, hematopoietic cancer, leukemia, lymphatic cancer, renal cancer, breast cancer (e.g. carcinoma), esophageal cancer, pancreatic cancer, skin cancer (e.g. basal cell carcinoma, squamous cell carcinoma, malignant melanoma, etc.), soft tissue cancer (e.g. angiosarcoma, leimyosarcoma, liposarcoma, rhabdomyosarcoma, myxoma, malignant fibrous histiocytoma-pleomorphic sarcoma, etc.), testicular cancer (e.g., germinoma, seminoma, etc.), thyroid cancer (e.g., anaplastic carcinoma, follicular carcinoma, papillary carcinoma, Hurthle cell carcinoma, etc.), bladder cancer (e.g. transitional cell carcinoma), cervical cancer (e.g. adenocarcinoma), uterine cancer, peritoneal cancer, brain cancer, neuroblastoma, mesothelioma, cholangiocarcinoma, chondrosarcoma, leukemia (e.g. AML, CML, CMML, JMML, etc.), lymphoma (e.g. ALL, Burkitt's lymphoma, Hodgkin's lymphoma, Plasma cell myeloma, etc.), adrenal cortical cancer, anal cancer, aplastic anemia, bile duct cancer, bone cancer, bone metastasis, brain cancers, central nervous system (CNS) cancers, peripheral nervous system (PNS) cancers, cervical cancer, childhood Non-Hodgkin's lymphoma, pancreatic cancer (e.g. ductal adenocarcinoma, endocrine tumor, etc.), colon cancer (e.g. adenocarcinoma, adenoma, etc.), colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's lymphoma, Kaposi's sarcoma, kidney cancer, laryngeal and pharyngeal cancer, acute lymphocytic leukemia, acute myeloid leukemia, children's leukemia, chronic lymphocytic leukemia, chronic myeloid leukemia, liver cancer (e.g. hepatocellular carcinoma), lung carcinoid tumors, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, penile cancer, pituitary tumor, prostate cancer (e.g. adenocarcinoma), retinoblastoma, rhabdomyosarcoma, salivary gland cancer, sarcomas, melanoma skin cancer, nonmelanoma skin cancers, stomach cancer (e.g. adenocarcinoma, etc.), thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, Waldenstrom's macroglobulinemia, ganglia cancer (e.g. neuroblastoma), or a nerve sheath cancer.

### F. Expression of CRISPR Effector Protein and CRISPR Polynucleotide

In some cases, one or more expression vectors for expressing CRISPR polynucleotide and CRISPR effector protein (e.g., CRISPR enzyme) can be transfected into a host cells. The expression vector comprising a DNA sequence coding for the CRISPR polynucleotide can be transfected into the host cell first and then an expression vector comprising a DNA sequence coding for the CRISPR effector protein (e.g., CRISPR enzyme) can be transfected into the host cell. The expression vector comprising a DNA sequence coding for the CRISPR effector protein (e.g., CRISPR enzyme) and an expression vector comprising a DNA sequence coding for the inducible CRISPR polynucleotide can be transfected simultaneously into the host cell. A single (type of) expression vector comprising a DNA sequence coding for the CRISPR effector protein (e.g., CRISPR enzyme) and a DNA sequence coding for the inducible CRISPR polynucleotide can be transfected into the host cell. The host cell can be a host cell which endogenously expresses the CRISPR effector protein (e.g., CRISPR enzyme). A messenger RNA encoding the CRISPR effector protein (e.g., CRISPR enzyme) can also be used with a CRISPR polynucleotide, e.g., a sgRNA for gene editing. When a vector is used, it can contain an inducible promoter. Conditional promoter(s) and/or inducible promoter(s) and/or tissue specific promoter(s) can be RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the j-actin promoter, the phosphoglycerol kinase (PGK) promoter, and the EF1α promoter. In some cases, a transgene encoding a CRISPR effector protein (e.g., CRISPR enzyme) can be integrated into a genome of cell.

A transgene expressing the CRISPR effector protein (e.g., CRISPR enzyme) can be introduced in a cell. A CRISPR effector protein (e.g., CRISPR enzyme, e.g., Cas9) transgene can be introduced into an isolated cell. A CRISPR complex transgenic cell can be obtained by isolating cells from a transgenic organism. A CRISPR effector protein (e.g., CRISPR enzyme, e.g., Cas9) transgene can be delivered to a eukaryotic cell by means of vector (e.g., AAV, adenovirus, lentivirus) and/or particle and/or nanoparticle delivery, as also described herein.

In some cases, the CRISPR polynucleotide can be inducibly expressed. In some cases, the CRISPR effector protein (e.g., CRISPR enzyme) can be inducibly expressed. Inducing expression of the CRISPR polynucleotide and/or CRISPR effector protein (e.g., CRISPR enzyme) can result in formation of a CRISPR polynucleotide/CRISPR effector protein (e.g., CRISPR enzyme) complex that can be turned “on” at a desired time to target a target nucleic acid (e.g., target DNA) and to cleave that target nucleic acid (e.g., target DNA). The inducible complexes can be used to reduce off-target effects by limiting the active half-life of the complex or by achieving tissue-specific editing in model organisms or in human cells.

The inducible CRISPR polynucleotide and/or CRISPR effector protein (e.g., CRISPR enzyme) can be expressed within a host cell. The expression can be in any order.

### V. CRISPR Polynucleotide Synthesis

The polynucleotide modified with an unnatural nucleotide for crosslinking further comprising a CRISPR ON polynucleotide sequence, CRISPR OFF polynucleotide sequence, CRISPR ON/OFF polynucleotide sequence, or CRISPR polynucleotides comprising one or more modifications that, when complexed with a CRISPR enzyme, have reduced off-target editing activity, described herein can be synthesized by any method known to one of ordinary skill in the art. The polynucleotide modified with an unnatural nucleotide for crosslinking further comprising a CRISPR ON polynucleotide, CRISPR OFF polynucleotide, or CRISPR ON/OFF polynucleotide, described herein can be chemically synthesized. The polynucleotide modified with an unnatural nucleotide for crosslinking further comprising a CRISPR ON polynucleotide, CRISPR OFF polynucleotide, or CRISPR ON/OFF polynucleotide, described herein can be synthesized using 2-0-thionocarbamate-protected nucleoside phosphoramidites. Methods of synthesis of polynucleotides are described in, e.g., Dellinger et al., J. American Chemical Society 133, 11540-11556 (2011); Threlfall et al., Organic & Biomolecular Chemistry 10, 746-754 (2012); and Dellinger et al, J. American Chemical Society 125, 940-950 (2003). Any of the modifications described herein can be combined and incorporate a CRISPR ON polynucleotide, CRISPR OFF polynucleotide, or CRISPR ON/OFF polynucleotide described herein, for example, in the guide sequence and/or the sequence that binds a CRISPR effector protein (e.g., scaffold sequence). Alternatively, the CRISPR polynucleotides can be prepared by the phosphoramidite method described by Beaucage and Caruthers (Tetrahedron Lett., (1981) 22:1859-1862), or by the triester method according to Matteucci, et al., (J. Am. Chem. Soc, (1981) 103:3185), each of which is specifically incorporated herein by reference, or by other chemical methods using a commercial automated polynucleotide synthesizer.

The CRISPR polynucleotides can be chemically synthesized, e.g., according to the solid phase phosphoramidite triester method first described by Beaucage and Caruthers, Tetrahedron Lett. 22: 1859-1862 (1981), using an automated synthesizer, as described in Van Devanter et. al, Nucleic Acids Res. 12:6159-6168 (1984). Synthesis of the CRISPR polynucleotides can comprise introducing chemical modifications that employ special phosphoramidite reagents during solid phase synthesis.

A. sgRNA Linkage

A CRISPR polynucleotide that is a sgRNA can comprise a modified crRNA and tracrRNA sequence chemically linked or conjugated via a non-phosphodiester bond. The modified crRNA and tracrRNA sequence can be chemically linked or conjugated via a non-nucleotide loop. The modified crRNA and tracrRNA can be joined via a non-phosphodiester covalent linker. The covalent linker can be a chemical moiety selected from the group consisting of coumarin, carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

### B. Cleavable Elements

The cleavable elements in the CRISPR polynucleotide herein can be provided with functional groups at each end that can be suitably protected or activated. The functional groups can be covalently attached via an ether, ester, carbamate, phosphate ester or amine linkage. For example, hexaethyleneglycol can be protected on one terminus with a photolabile protecting group (i.e., NVOC or MeNPOC) and activated on the other terminus with 2-cyanoethyl-N,N-diisopropylamino-chlorophosphite to form a phosphoramidite. Other methods of forming ether, carbamate or amine linkages are known to those of skill in the art and particular reagents and references can be found in such texts as March, Advanced Organic Chemistry, 4th Ed., Wiley-Interscience, New York, N.Y., 1992.

Methods of synthesizing the linkers described herein are well-known in the art. A non-limiting example of a method of synthesizing a linker of this application is provided below:

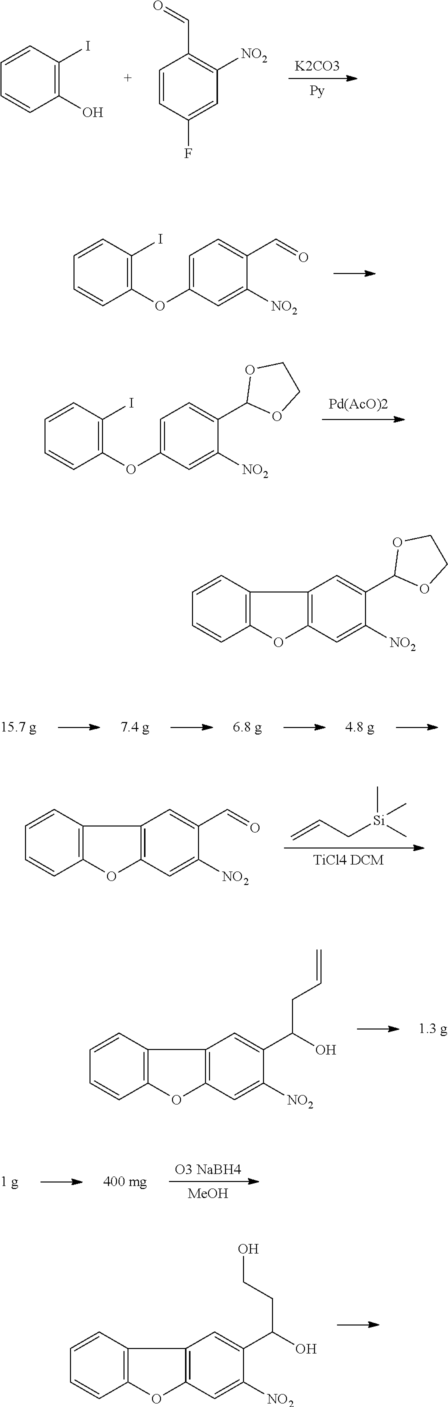

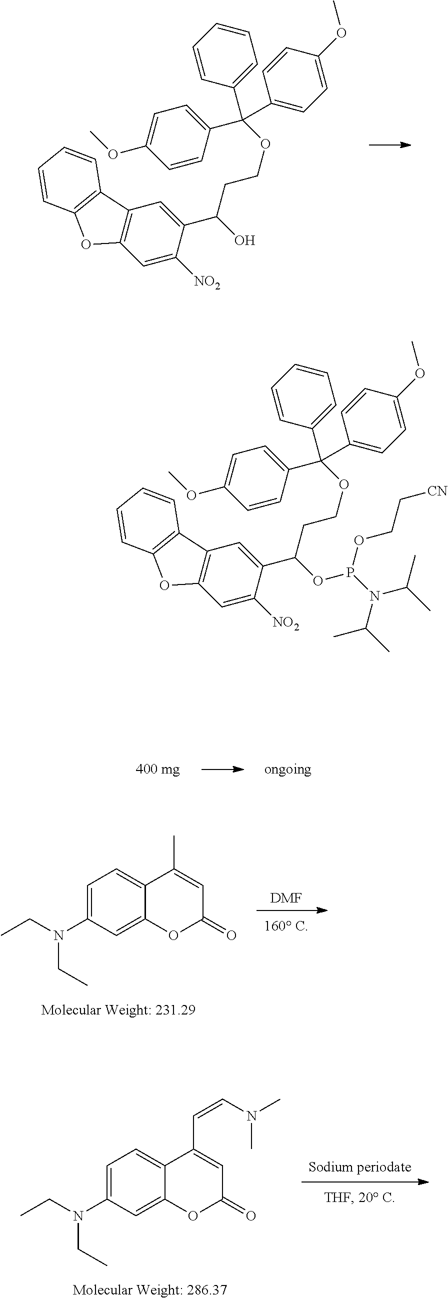

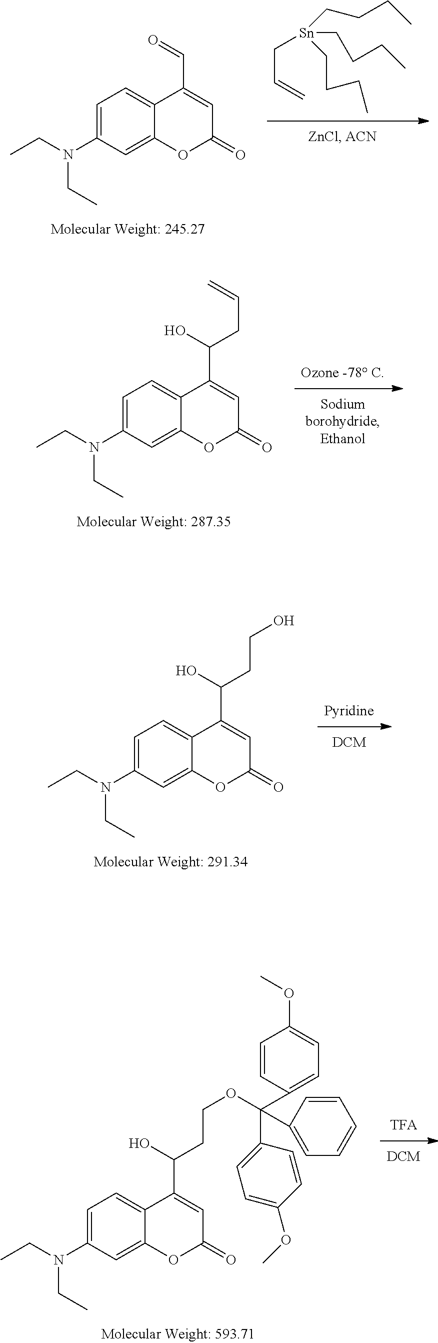 [SMILES: CCN(CC)c1ccc2c(C=O)cc(=O)oc2c1 ; C=C[CH2][Sn]([CH2]CCC)([CH2]CCC)[CH2]CCC ; C=CCC(O)c1cc(=O)oc2cc(N(CC)CC)ccc12 ; CCN(CC)c1ccc2c(C(O)CCO)cc(=O)oc2c1 ; CCN(CC)c1ccc2c(C(O)CCOC(c3ccccc3)(c3ccc(OC)cc3)c3ccc(OC)cc3)cc(=O)oc2c1]

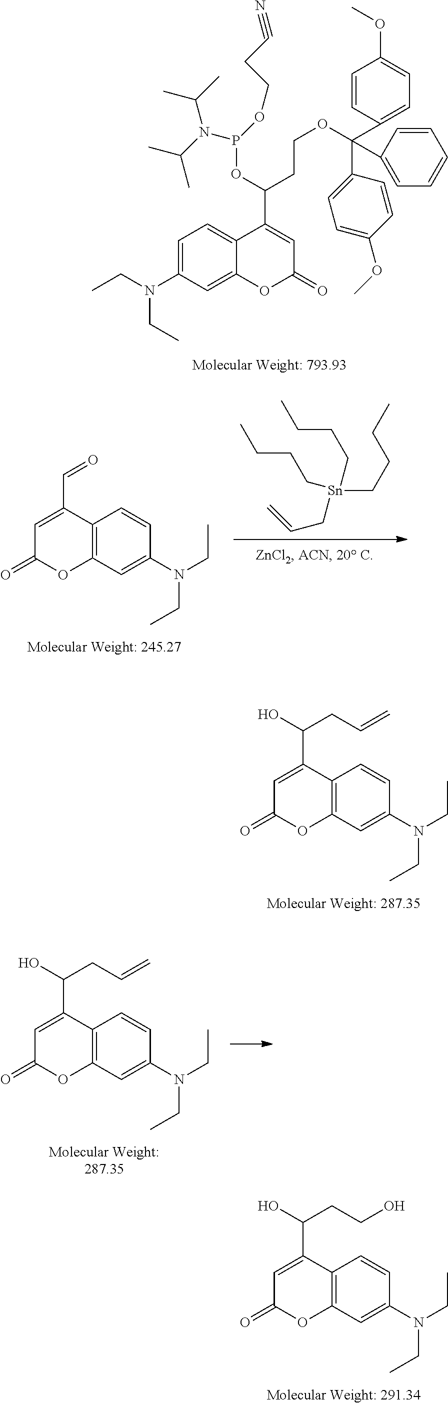 [SMILES: CCN(CC)c1ccc2c(C(CCOC(c3ccccc3)(c3ccc(OC)cc3)c3ccc(OC)cc3)OP(OCCC#N)N(C(C)C)C(C)C)cc(=O)oc2c1 ; CCN(CC)c1ccc2c(C=O)cc(=O)oc2c1 ; C=C[CH2][Sn]([CH2]CCC)([CH2]CCC)[CH2]CCC ; C=CCC(O)c1cc(=O)oc2cc(N(CC)CC)ccc12 ; C=CCC(O)c1cc(=O)oc2cc(N(CC)CC)ccc12 ; CCN(CC)c1ccc2c(C(O)CCO)cc(=O)oc2c1]

Further non-limiting examples of cleavable linkers are methods of synthesizing said linkers are described below:

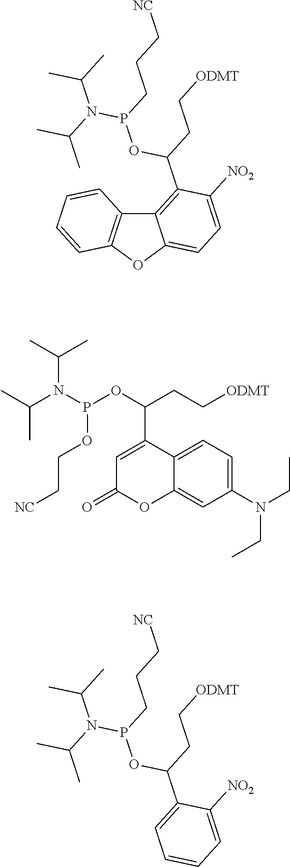

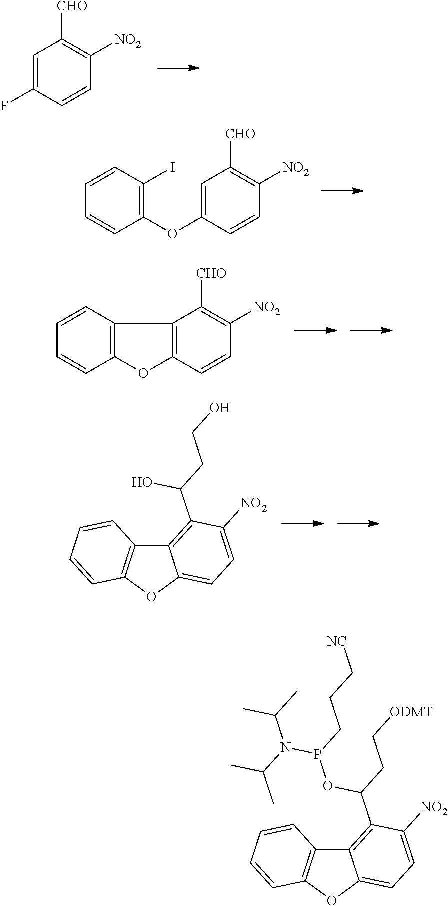

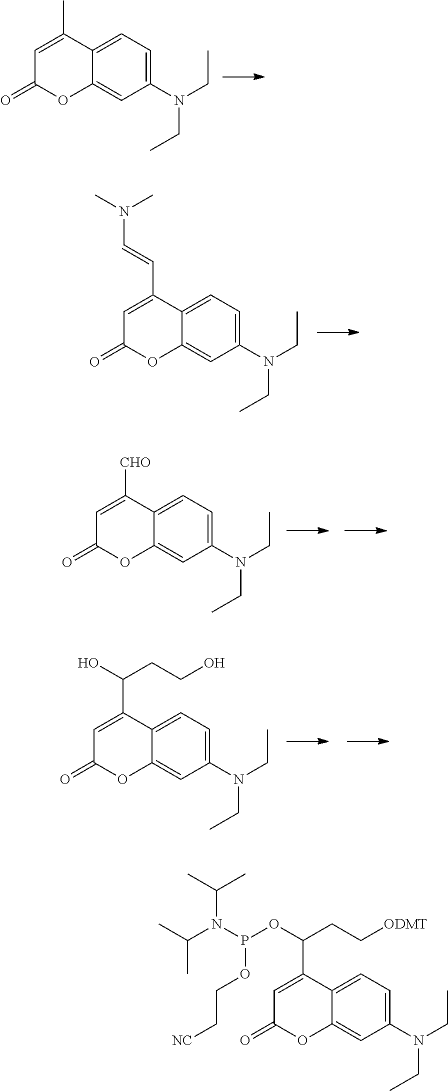

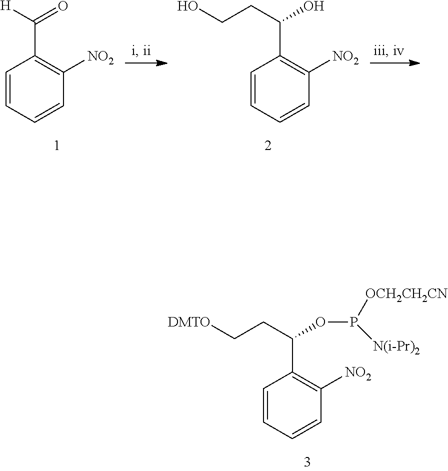

Non-limiting examples of cleavable linkers are methods of synthesizing said linkers are described below:

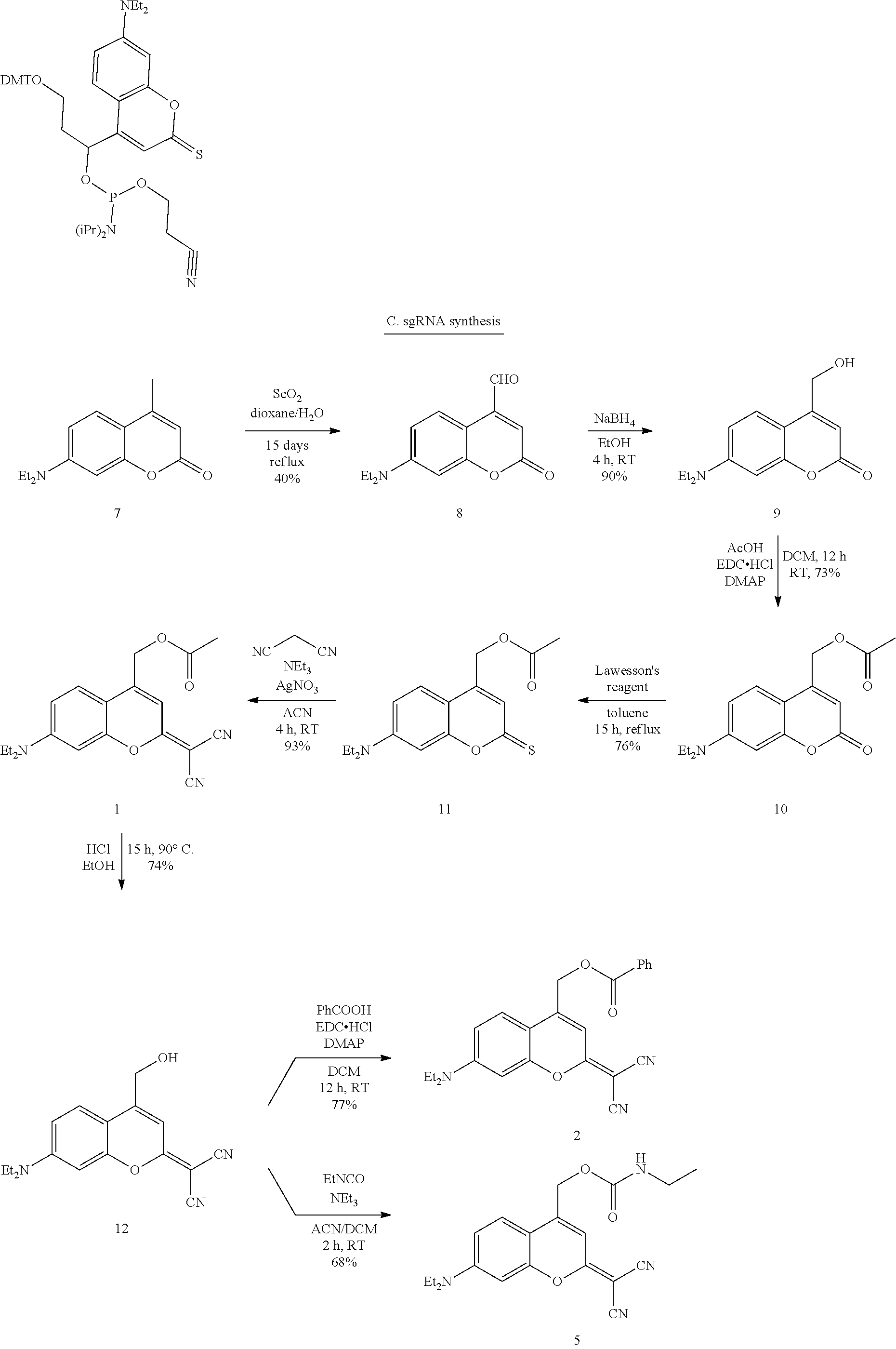

The sgRNA comprising crRNA and tracrRNA can first be synthesized using a phosphoramidite synthetic protocol (Herdewijn, P., ed., Methods in Molecular Biology Col 288, Polynucleotide Synthesis: Methods and Applications, Humana Press, New Jersey (2012)). The sgRNA comprising crRNA and tracrRNA sequences can be functionalized to contain an appropriate functional group for ligation (see e.g., Hermanson, G. T., Bioconjugate Techniques, Academic Press (2013)). The functional group can be hydroxyl, amine, carboxylic acid, carboxylic acid halide, carboxylic acid active ester, aldehyde, carbonyl, chlorocarbonyl, coumarin, psoralen, diazirine, or azide. Once the modified tracr and the tracr mate sequences are functionalized, a covalent chemical bond or linkage can be formed between the two polynucleotides. The chemical bonds can be based on coumarin, carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

The sgRNA comprising crRNA and tracrRNA sequence and can be chemically synthesized. The sgRNA can be synthesized together in the form of a fusion or synthesized separately and chemically linked. The chemical synthesis can use automated using solid-phase polynucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

The sgRNA can be covalently linked with various bioconjugation reactions, loops, bridges, and non-nucleotide links via modifications of sugar, internucleotide phosphodiester bonds, purine and pyrimidine residues. Sletten et al., Angew. Chem. Int. Ed. (2009) 48:6974-6998; Manoharan, M. Curr. Opin. Chem. Biol. (2004) 8: 570-9; Behlke et al., Polynucleotides (2008) 18: 305-19; Watts, et al., Drug. Discov. Today (2008) 13: 842-55; Shukla, et al., ChemMedChem (2010) 5: 328-49.

The sgRNA can be assembled using click chemistry. The crRNA tracrRNA and/or the sequence elements therein can be assembled by covalent linkage using a triazole linker. The sgRNA can be covalently linked by ligating a 5′-hexyne tracrRNA and a 3′-azide crRNA. Either or both of the 5′-hexyne tracrRNA and a 3′-azide crRNA can be protected with 2′-acetoxyethl orthoester (2′-ACE) group, which can be subsequently removed using Dharmacon protocol (Scaringe et al., J. Am. Chem. Soc. (1998) 120: 11820-11821; Scaringe, Methods Enzymol. (2000) 317: 3-18).

### VI. Kits

A kit can comprise one or more of the components described herein. The kit can comprise a CRISPR polynucleotide described herein. The kit can comprise a CRISPR effector protein (e.g., a CRISPR enzyme, e.g., Cas9) described herein. The kit can comprise a CRISPR complex described herein comprising a CRISPR polynucleotide described herein and a CRISPR effector protein described herein. The kit can comprise a linker, for example a cleavable linker. The kit can comprise a photocleavable linker. The kit can comprise instructions. The kit can comprise a cell or organism comprising a CRISPR polynucleotide, CRISPR effector protein, or CRISPR complex described herein.

The kit can comprise a genetic construct, e.g., vector system for expressing one or more CRISPR polynucleotides and/or one or more CRISPR effector proteins and instructions for using the kit. The kit can comprise a cell that comprises one or more genetic constructs (e.g., one or more vector systems) for expressing a CRISPR polynucleotide and/or CRISPR effector protein described herein.

The kit can comprise an excipient to generate a composition suitable for contacting a nucleic acid target with e.g., a CRISPR complex described herein. The composition can be suitable for contacting a nucleic acid target sequence within a genome. The composition can be suitable for delivering the composition (e.g., a CRISPR polynucleotide, e.g., a sgRNA, e.g., complexed with a CRISPR effector protein, e.g., a CRISPR enzyme, e.g. Cas9) to a cell. The composition can be suitable for delivering a CRISPR polynucleotide, e.g., a gRNA, or complexes thereof with CRISPR enzyme) to a subject. The excipient can be a pharmaceutically acceptable excipient.

The kit can comprise one or more reagents for use in cleaving one or more of the cleavable elements of the CRISPR polynucleotides described herein. The one or more reagents can be provided in any suitable container. The kit can comprise one or more reaction or storage buffers. The kit can comprise a reagent. The reagent can be provided in a form that is usable in a particular assay, or in a form that requires addition of one or more other components before use (e.g. in concentrate or lyophilized form). A reaction or storage buffer can be any buffer, e.g., sodium carbonate buffer, a sodium bicarbonate buffer, a borate buffer, a Tris buffer, a MOPS buffer, a HEPES buffer, or a combination thereof. The buffer can have a pH from about 7 to about 10.

The kit can comprise one or more polynucleotides corresponding to a guide sequence for insertion into a vector so as to operably link the guide sequence and a regulatory element. The kit can comprise a homologous recombination template polynucleotide.

### EXEMPLARY NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure numbered 1-244 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below.

### EXEMPLARY EMBODIMENTS

1. A CRISPR complex comprising a guide RNA (gRNA) conjugated to a CRISPR effector protein via a modified nucleotide within the gRNA, wherein the gRNA comprises a target binding region and a CRISPR effector protein binding region, and wherein the modified nucleotide is outside the target binding region.
2. The CRISPR complex of embodiment 1, wherein the gRNA is a single guide RNA (sgRNA) comprising a crRNA region comprising a target binding region and a tracrRNA region that comprises the CRISPR effector protein binding region, and wherein the modified nucleotide is within the tracrRNA region.
3. The CRISPR complex of embodiment 1 or 2, wherein the gRNA is conjugated to the CRISPR effector protein via the modified nucleotide conjugated to a cysteine residue in the CRISPR effector protein.
4. The CRISPR complex of embodiments 1 or 2, wherein the gRNA or the sgRNA is conjugated to the CRISPR effector protein via the modified nucleotide conjugated to a naturally occurring cysteine residue in the CRISPR effector protein.
5. The CRISPR complex of any one of embodiments 1 to 4, wherein the CRISPR effector protein is Cas9.
6. The CRISPR complex of embodiment 5, wherein the modified nucleotide is conjugated to a Cys80 in the Cas9 enzyme.
7. The CRISPR complex of embodiment 1 or 2, wherein the gRNA or the sgRNA is conjugated to the CRISPR effector protein via the modified nucleotide conjugated to a cysteine residue in the CRISPR effector protein, which cysteine residue is an amino acid substitution of a naturally occurring residue in the CRISPR effector protein.
8. The CRISPR complex of any of embodiments 1-7, wherein the gRNA or the sgRNA is conjugated to the CRISPR effector protein via the modified nucleotide comprising a maleimide moiety conjugated to a thiol of a cysteine residue in the CRISPR effector protein.
9. The CRISPR complex of any of embodiments 1-8, wherein the modified nucleotide is within 20 angstroms of a cysteine residue of the CRISPR effector protein when the gRNA or sgRNA binds to the CRISPR effector protein.
10. The CRISPR complex of embodiment 1 or 2, wherein the CRISPR effector protein comprises a non-proteogenic amino acid and the gRNA or sgRNA is conjugated to the CRISPR effector protein via the modified nucleotide conjugated to the non-proteogenic amino acid.
11. The CRISPR complex of embodiment 10, wherein the non-proteogenic amino acid is an azido-containing amino acid.
12. The CRISPR complex of any one of embodiments 1-11, wherein the modified nucleotide is not at the 3′ end of the sgRNA or at a position 5, 4, 3, 2, or 1 nucleotides from the 3′ end.
13. The CRISPR complex of any of embodiments 1-12, wherein the modified nucleotide is at one or more nucleotide positions: 22, 23, 24, 25, 31, 37, 44, 49, 45, 50, 56, 59, 63, 64, 66, 71, 72, 77, 78, 80, 84, 90 or 94 of the sgRNA, wherein nucleotide position 1 is at the 5′ end of the target binding region of the crRNA and nucleotide positions of the sgRNA are numbered consecutively from 5′ to 3′ from nucleotide position 1.
14. The CRISPR complex of any of embodiments 1-12, wherein the modified nucleotide is at nucleotide position 49 of the sgRNA, wherein nucleotide position 1 is at the 5′ end of the target binding region of the crRNA and nucleotide positions of the sgRNA are numbered consecutively from 5′ to 3′ from nucleotide position 1.
15. The CRISPR complex of any of embodiments 1-12, wherein the modified nucleotide is in a stem loop of the tracrRNA region.
16. The CRISPR complex of embodiment 15, wherein the structure of the stem loop is maintained relative to the structure of the stem loop in a sgRNA lacking the modified nucleotide.
17. The CRISPR complex of embodiments 1-12, wherein the modified nucleotide is in a bulge of the tracrRNA region.
18. The CRISPR complex of embodiment 17, wherein the structure of the bulge is maintained relative to the structure of the bulge in an sgRNA lacking the modified nucleotide.
19. The CRISPR complex of embodiment 1-12, wherein the modified nucleotide is between stem loops of the tracrRNA region.
20. The CRISPR complex of any of embodiments 1-19, wherein the modified nucleotide is a modified uracil nucleotide or a modified thymidine nucleotide.
21. The CRISPR complex of any of embodiments 1-20, wherein the modified nucleotide comprises a modified sugar moiety.
22. The CRISPR complex of any of embodiments 1-21, wherein the modified nucleotide comprises a modified base.
23. The CRISPR complex of any of embodiments 1-22, wherein the modified nucleotide comprises a maleimide moiety.
24. The CRISPR complex of any of embodiments 1-22, wherein the modified nucleotide comprises a N-hydroxysuccinimide (NHS) moiety.
25. The CRISPR complex of any of embodiments 1-22, wherein the modified nucleotide comprises a diazirine moiety.
26. The CRISPR complex of any of embodiments 1-22, wherein the modified nucleotide comprises a moiety selected from the group consisting of haloacetyl, imidoester, pyridyl disulfide, alkoxyamine, hydrazide, aryl azide, isocyanate, dithiol phosphoramidite DTPA, and dibenzocyclooctyne (DBCO).
27. The CRISPR complex of any of embodiments 1-22, wherein the modified nucleotide comprises 5′-dimethoxytrityl-5-[N-(trifluoroacetylaminohexyl)-3-acrylimido]-2′-deoxyuridine, 3′-[(2-cyanoethyl)-(N,N-diisopropyl)]-phosphoramidite.
28. The CRISPR complex of any of embodiments 1-22, wherein the modified nucleotide comprises 5′-dimethoxytrityl-5-[N-(4-maleimidobutyramido)hexyl)-3-acrylimido]-2′-deoxyuridine, 3′-[(2-cyanoethyl)-(N,N-diisopropyl)]-phosphoramidite.
29. The CRISPR complex of any of embodiments 1-22, wherein the modified nucleotide comprises 4-thio-UTP, 5-azido-UTP, 5-bromo-UTP, 8-azido-ATP, 5-APAS-UTP, or 8-N(3)AMP.
30. The CRISPR complex of any of embodiments 1-29, wherein the modified nucleotide is not 4-thiouridine or a modified adenosine.
31. The CRISPR complex of any of embodiments 1-30, wherein the CRISPR complex comprises nuclease activity.
32. The CRISPR complex of any of embodiments 1-30, wherein the CRISPR complex comprises nuclease activity that is specific for a target sequence corresponding to the target binding region of the CRISPR complex.
33. The CRISPR complex of embodiment 31 or 32, wherein an off-target nuclease activity of the CRISPR complex is equal to or less than an off-target nuclease activity of a CRISPR complex comprising the CRISPR effector protein and the gRNA that are not conjugated.
34. A CRISPR complex comprising a single guide RNA (sgRNA) conjugated to a CRISPR effector protein via a modified nucleotide at nucleotide position 49 of the sgRNA, wherein the sgRNA comprises a crRNA region comprising a target binding region and a tracrRNA region, and wherein the nucleotide position 1 is at a 5′ end of the target binding region of the crRNA and nucleotide positions of the sgRNA are numbered consecutively from 5′ to 3′ from nucleotide position 1.
35. The CRISPR complex of embodiment 34, wherein the modified nucleotide at nucleotide position 49 is a modified uracil nucleotide or a modified thymidine nucleotide.
36. The CRISPR complex of embodiment 34 or 35, wherein the CRISPR complex comprises nuclease activity.
37. The CRISPR complex of any of embodiments 34-36, wherein the CRISPR complex comprises nuclease activity that is specific for a target sequence corresponding to the target binding region of the CRISPR complex.
38. The CRISPR complex of any of embodiments 34-37, wherein an off-target nuclease activity of the CRISPR complex is equal to or less than an off-target nuclease activity of a CRISPR complex comprising the CRISPR effector protein and the gRNA that are not conjugated.
39. The CRISPR complex of any one of embodiments 1 to 38, wherein the gRNA or the sgRNA does not comprise a chemical modification at the 3′ and/or the 5′ end or at a position within 5, 4, 3, 2, or 1 nucleotide from the 3′ and/or the 5′ end.
40. The CRISPR complex of any one of embodiments 1 to 38, wherein the gRNA or the sgRNA comprises a chemical modification at the 3′ and/or the 5′ end or at a position within 5, 4, 3, 2, or 1 nucleotide from the 3′ and/or the 5′ end.
41. The CRISPR complex of any one of embodiments 1 to 40, wherein the gRNA or sgRNA further comprises a sequence configured to modulate activity of the CRISPR complex.
42. The CRISPR complex of embodiment 41, wherein the gRNA or the sgRNA comprises a CRISPR ON polynucleotide, a CRISPR OFF polynucleotide or a CRISPR ON/OFF polynucleotide comprising a cleavable linker.
43. A single guide (sgRNA) comprising a crRNA region comprising a target binding region and a tracrRNA that comprises a CRISPR effector protein binding region, wherein the tracrRNA region comprises a modified nucleotide that conjugates with the CRISPR effector protein when the sgRNA binds to the CRISPR effector protein.
44. A sgRNA of embodiment 43, wherein the tracrRNA region comprises a modified nucleotide comprising a moiety that conjugates with a thiol group of the CRISPR effector protein.
45. The sgRNA of embodiment 42 or 43, wherein sgRNA conjugates to the CRISPR effector protein via the modified nucleotide conjugating to a naturally occurring cysteine residue in the CRISPR effector protein.
46. The sgRNA of any one of embodiments 43-45, wherein the CRISPR effector protein is Cas9.
47. The sgRNA of any one of embodiments 43-46, wherein the modified nucleotide conjugates to a Cys80 in the Cas9 enzyme.
48. The sgRNA of any one of embodiments 43-46, wherein the modified nucleotide is not at the 3′ end of the sgRNA or within 5, 4, 3, 2, or 1 nucleotide from the 3′ end.
49. The sgRNA of any of embodiments 43-48, wherein the modified nucleotide is at one or more nucleotide positions: 22, 23, 24, 25, 31, 37, 44, 49, 45, 50, 56, 59, 63, 64, 66, 71, 72, 77, 78, 80, 84, 90 or 94 of the sgRNA, wherein nucleotide position 1 is at the 5′ end of the target binding region of the crRNA and nucleotide positions of the sgRNA are numbered consecutively from 5′ to 3′ from nucleotide position 1.
50. The sgRNA of any of embodiments 43-48, wherein the modified nucleotide is at nucleotide position 49 of the sgRNA, wherein nucleotide position 1 is at the 5′ end of the target binding region of the crRNA and nucleotide positions of the sgRNA are numbered consecutively from 5′ to 3′ from nucleotide position 1.
51. The sgRNA of any of embodiments 43-48, wherein the modified nucleotide is in a stem loop of the tracrRNA region.
52. The sgRNA of embodiment 51, wherein the structure of the stem loop is maintained relative to the structure of the stem loop in a sgRNA lacking the modified nucleotide.
53. The sgRNA of any of embodiments 43-48, wherein the modified nucleotide is in a bulge of the tracrRNA region.
54. The sgRNA of embodiment 53, wherein the structure of the bulge is maintained relative to the structure of the bulge in an sgRNA lacking the modified nucleotide.
55. The sgRNA of any of embodiments 43-48, wherein the modified nucleotide is between stem loops of the tracrRNA region.
56. The sgRNA of any of embodiments 43-55, wherein the modified nucleotide is a modified uracil nucleotide or a modified thymidine nucleotide.
57. The sgRNA of any of embodiments 43-56, wherein the modified nucleotide comprises a modified sugar moiety.
58. The sgRNA of any of embodiments 43-57, wherein the modified nucleotide comprises a modified base.
59. The sgRNA of any of embodiments 43-58, wherein the modified nucleotide comprises a maleimide moiety.
60. The sgRNA of any of embodiments 43-58, wherein the modified nucleotide comprises a N-hydroxysuccinimide (NHS) moiety.
61. The sgRNA of any of embodiments 43-58, wherein the modified nucleotide comprises a diazirine moiety.
62. The sgRNA of any of embodiments 43-58, wherein the modified nucleotide comprises a moiety selected from the group consisting of haloacetyl, imidoester, pyridyl disulfide, alkoxyamine, hydrazide, aryl azide, isocyanate, dithiol phosphoramidite DTPA, and dibenzocyclooctyne (DBCO).
63. The sgRNA of any of embodiments 43-58, wherein the modified nucleotide comprises 5′-dimethoxytrityl-5-[N-(trifluoroacetylaminohexyl)-3-acrylimido]-2′-deoxyuridine, 3′-[(2-cyanoethyl)-(N,N-diisopropyl)]-phosphoramidite.
64. The sgRNA of any of embodiments 43-58, wherein the modified nucleotide comprises 5′-dimethoxytrityl-5-[N-(4-maleimidobutyramido)hexyl)-3-acrylimido]-2′-deoxyuridine, 3′-[(2-cyanoethyl)-(N,N-diisopropyl)]-phosphoramidite.
65. The sgRNA of any of embodiments 43-58, wherein the modified nucleotide comprises 4-thio-UTP, 5-azido-UTP, 5-bromo-UTP, 8-azido-ATP, 5-APAS-UTP, or 8-N(3)AMP.
66. The sgRNA of any of embodiments 43-65, wherein the modified nucleotide is not 4-thiouridine or a modified adenosine.
67. The sgRNA of any of embodiments 43-66, wherein the modified nucleotide does not comprise a 2′-O-methyl moiety or a phosphorothioate moiety.
68. The sgRNA of any one of embodiments 43-66, wherein the sgRNA does not comprise a chemical modification at the 3′ and/or the 5′ end or at a position within 5, 4, 3, 2, or 1 nucleotide from the 3′ and/or the 5′ end.
69. The sgRNA of any one of embodiments 43-66, wherein the sgRNA comprises a chemical modification at the 3′ and/or the 5′ end or at a position within 5, 4, 3, 2, or 1 nucleotide from the 3′ and/or the 5′ end.
70. The sgRNA of any one of embodiments 43-69, further comprising a sequence configured to modulate activity of the CRISPR complex.
71. The sgRNA of embodiment 70, wherein the sgRNA comprises a CRISPR ON polynucleotide, a CRISPR OFF polynucleotide or a CRISPR ON/OFF polynucleotide comprising a cleavable linker.
72. A pharmaceutical formulation comprising one or more of the CRISPR complex of any one of embodiments 1 to 42 or the sgRNA of any one of embodiments 43-71.
73. The pharmaceutical formulation of embodiment 72 further comprising a pharmaceutically acceptable excipient.
74. A method comprising administering the pharmaceutical formulation of embodiment 72 or 73 to a subject.
75. The method of embodiment 74, wherein the subject is a mammal.
76. The method of embodiment 75, wherein the mammal is a human.
77. A method comprising introducing one or more of the CRISPR complex of any one of embodiments 1 to 42 and/or the sgRNA of any one of embodiments 43-71 into a cell.
78. The method of embodiment 77, wherein the cell is a stem cell.
79. The method of embodiment 78, wherein the cell is an induced pluripotent stem cell.
80. The method of embodiment 77, wherein the cell is an immune cell.
81. The method of embodiment 80, wherein the immune cell is a T cell.
82. The method of embodiment 80, wherein the immune cell is a natural killer cell (NK cell).
83. A method of editing a target nucleic acid molecule comprising contacting the CRISPR complex of any one of embodiments 1 to 42 with the target nucleic acid molecule.
84. The method of embodiment 83, wherein the CRISPR complex comprises an off-target cleavage activity of less than 2% of cleavage events.
85. A method of editing a target gene in one or more cells comprising administering the CRISPR complex of any one of embodiments 1 to 42 to the one or more cells comprising the target gene, thereby editing the target gene in the one or more cells.
86. A method of producing a CRISPR complex comprising a guide RNA (gRNA) conjugated to a CRISPR effector protein via a modified nucleotide within the gRNA, wherein the gRNA comprises a target binding region and a CRISPR effector protein binding region, and wherein the modified nucleotide is outside the target binding region, and the method comprises reacting the gRNA and the CRISPR effector protein under conditions whereby the modified nucleotide conjugates the gRNA to the CRISPR effector protein.
87. The method of embodiment 86, wherein the gRNA is a single guide RNA (sgRNA)
88. The method of embodiment 86 or 87, wherein nuclease activity of the CRISPR effector protein is maintained after conjugation.
89. The method of embodiment 70, wherein reacting is performed in a solution and the ratio of the gRNA or sgRNA to the CRISPR effector protein in the solution is at least 9:1.
90. The method of any of embodiments 86-89, wherein reacting comprises exposing the solution to ultraviolet (UV) light.
91. The method of any of embodiments 86-90, wherein reacting comprises contacting the gRNA or the sgRNA and the CRISPR effector protein.
92. A system comprising one or more of the CRISPR complex of any one of 1 to 42 and/or the sgRNA of any one of embodiments 43-71.
93. A kit comprising as a component or as components one or more of the CRISPR complexes of any one one of 1 to 42 and/or the sgRNA of any one of embodiments 43-71.
94. The kit of embodiment 93 further comprising additional components or instructions for carrying out methods of any one of embodiments 74-91.

### EXAMPLES

### Example 1: Modified Polynucleotide with Cross-Linker Positioned in a Tetraloop

Initial studies create a modified polynucleotide wherein the specific nucleotide at position 22 and position 49 are switched. As can be seen in FIG. 1, the nucleotide is located at the base of the tetraloop. Further, the nucleotides at this position are converted to deoxyribonucleic acids. This conversion has minimal effects to polynucleotide or ribonucleoprotein activity FIG. 2. Alternatively, the uracil molecule at position 50 can be converted to deoxythymidine.

Polynucleotides are next further modified by substituting the deoxynucleotide at either position 49 or 50 with an analog containing a reactive linker compound. To this reactive linker, a maleimide is covalently attached through known chemistry. Additionally, this maleimide compound can contain a variable length spacer. Modified polynucleotides are then mixed with Cas9 to form ribonucleoproteins. Ribonucleoproteins will undergo a conjugating reaction under physiological conditions.

Conjugated ribonucleoproteins (Locked RNPs) are next purified from individual free components and non-specific conjugated species. Mixed solutions are first purified using size exclusion chromatography to separate free polynucleotides. Collected fractions are next passed through an affinity chromatography column with immobilized sgRNAs to separate free polypeptides from formed RNPs. Finally, purified RNPs are passed through a cation exchange column in high salt conditions (e.g., 300 mM NaCl) to isolate locked RNPs. High salt conditions will cause dissociation of RNPs into component species Cas9 and sgRNA. Due to the covalently bond nature of locked RNPs, these species will stay together and can be purified accordingly.

Purified locked RNPs are transfected into immortalized cell lines to assay their capability to form double strand breaks within human cells. Polynucleotides to target specific regions of the genome are synthesized and formed into locked RNPs. Following transfection, Sanger sequencing is performed on transfected pool and resulting genomic data is analyzed by Inference of CRISPR Edits (ICE) software to detect the presence of indel mutations.

To test the specificity of the locked RNP complex, locked RNPs will be formed containing a specific polynucleotide sequence and purified. To this purification, a 10-fold excess of polynucleotide targeting a separate genomic region will be added and allowed to mix. This mixture is then transfected into cells. Editing at both loci is analyzed and shows that CRISPR induced editing only occurs at the locus targeted by locked RNPs. Sample gRNAs and sample targeting regions are provided in the attached Sequence Listing which is incorporated herein by reference.

### Example 2: Construction of Single Guide RNA Comprising Modified Nucleotide for Conjugation to Cas9

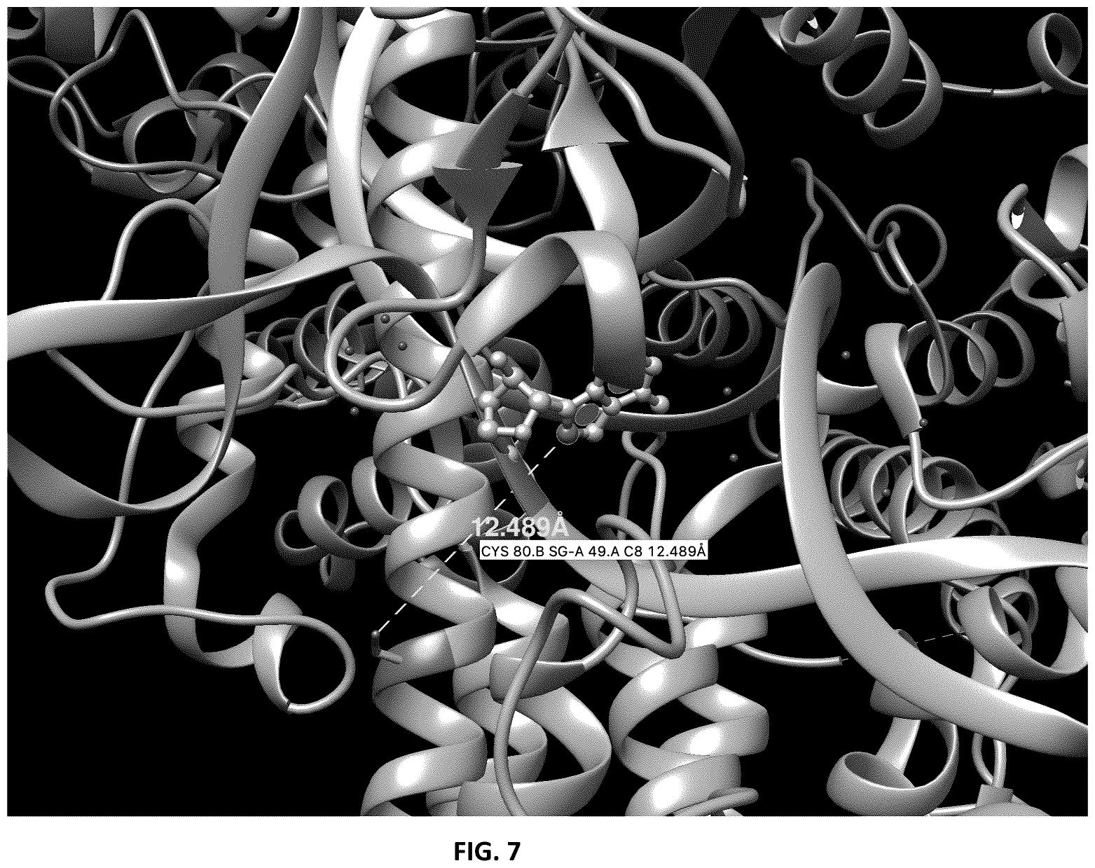

A single guide RNA (sgRNA) was constructed comprising a maleimide-modified nucleotide for conjugation to Cas9. Conjugation is based on a reaction between a free thiol on a cysteine residue of Cas9 and the maleimide moiety of the maleimide-modified nucleotide of the sgRNA. Cas9 contains two cysteines (Cys-80 and Cys574). Cys-80 is located adjacent to A49 of a sgRNA (e.g., a standard 100-mer sgRNA) when Cas9 and the sgRNA form a complex. (See FIG. 7). Therefore, we designed a sgRNA having a maleimide-modified nucleotide at position 49 which can be prepared via a modified amidite intermediate.

In natural systems, sgRNA comprises a U22-A49 basepair. However, modified adenine amidites are relatively expensive. Because modified uracil amidites and especially modified uracil amidites are more readily available, we have swapped the pair U22-A49 in the lower stem of sgRNA hairpin to create a derivative having an A22-T49 pair. We then verified that an sgRNA with the swapped A22-T49 pair supports editing via CRISPR. From there, we modify the thymidine nucleotide with desired linkers to conjugate the sgRNA to Cas9. This approach of swapping the relative location of hybridized bases, (or more generally, replacing an X-Y pair with an A-T pair) in an RNA hairpin of sgRNA can be utilized in similar situations where A, C, or G are located in proximity to a desired cysteine in a nuclease and it is desirable to swap the A, C, or G with a modified thymidine (or uracil).

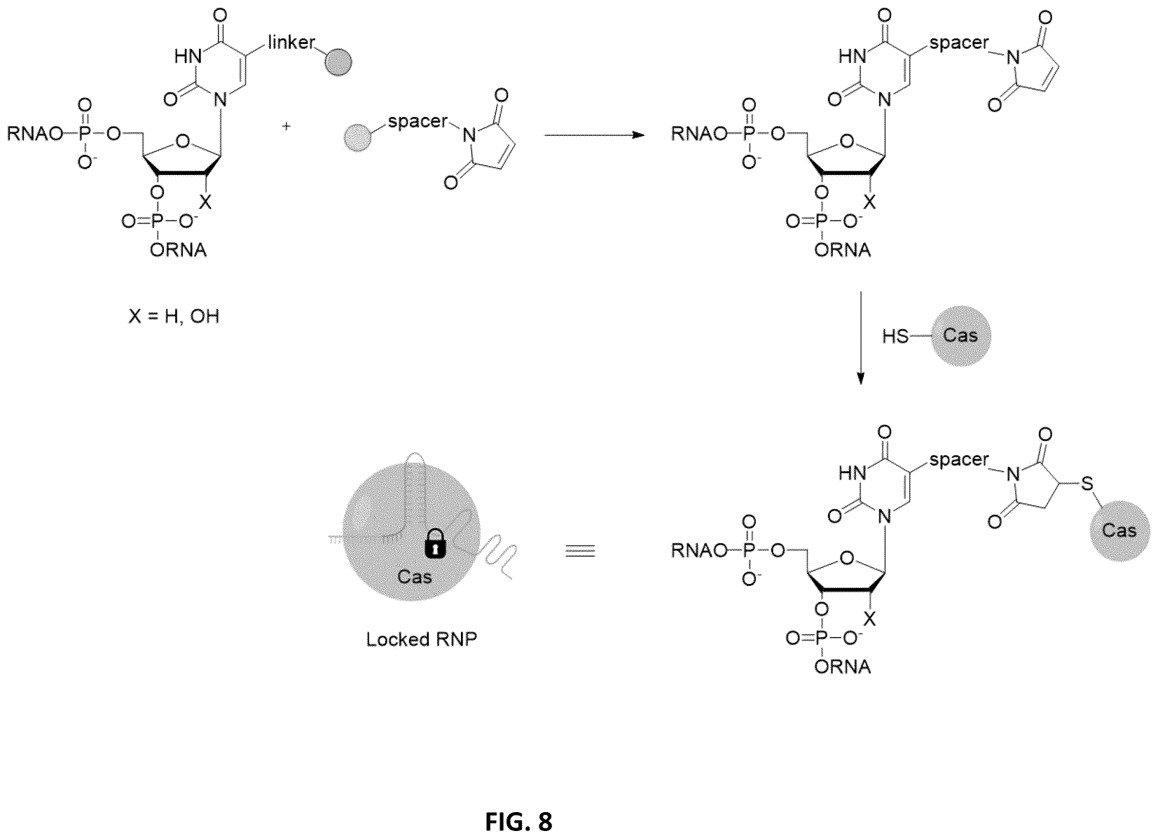

FIG. 8 illustrates a general reaction scheme for modifying a thymidine or uracil of sgRNA to comprise a maleimide for conjugation to a thiol group of a cysteine residue of Cas to prepare a locked sgRNA/Cas complex.

FIG. 9 illustrates a standard sgRNA 100mer sequence and a modified sequence in which the U22-A49 pair has been swapped for an A22-Y49 pair. Y is 5′-Dimethoxytrityl-5-[N-(trifluoroacetylaminohexyl)-3-acrylimido]-2′-deoxyUridine, 3′-[(2-cyanoethyl)-(N,N-diisopropyl)]-phosphoramidite.

FIG. 10 provides an illustration of a standard sgRNA sequence and a modified sequence in which the U22-A49 pair has been swapped for an A22-dT49 pair.

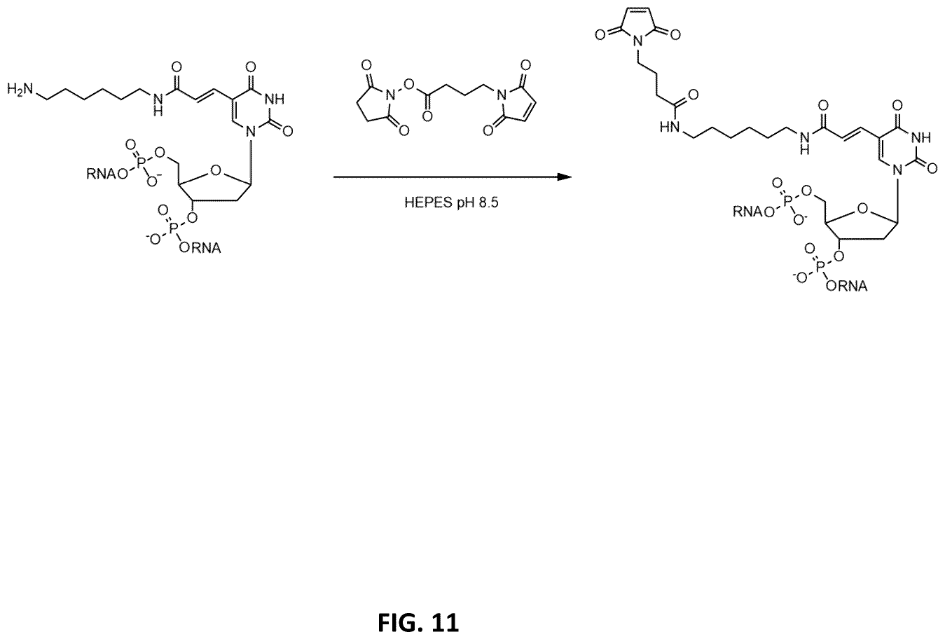

FIG. 11 provides a reaction scheme using a heterobifunctional crosslinker to install maleimide moiety in a nucleotide of sgRNA.

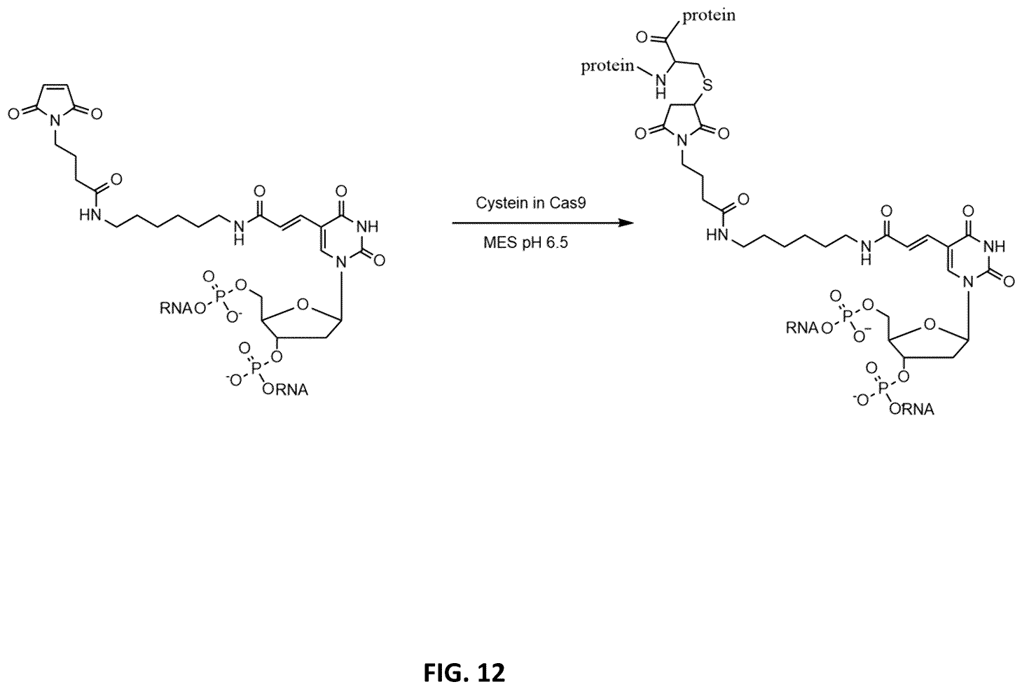

FIG. 12 provides a reaction scheme between a maleimide-modified nucleotide of a sgRNA and a cysteine redicue of Cas9.

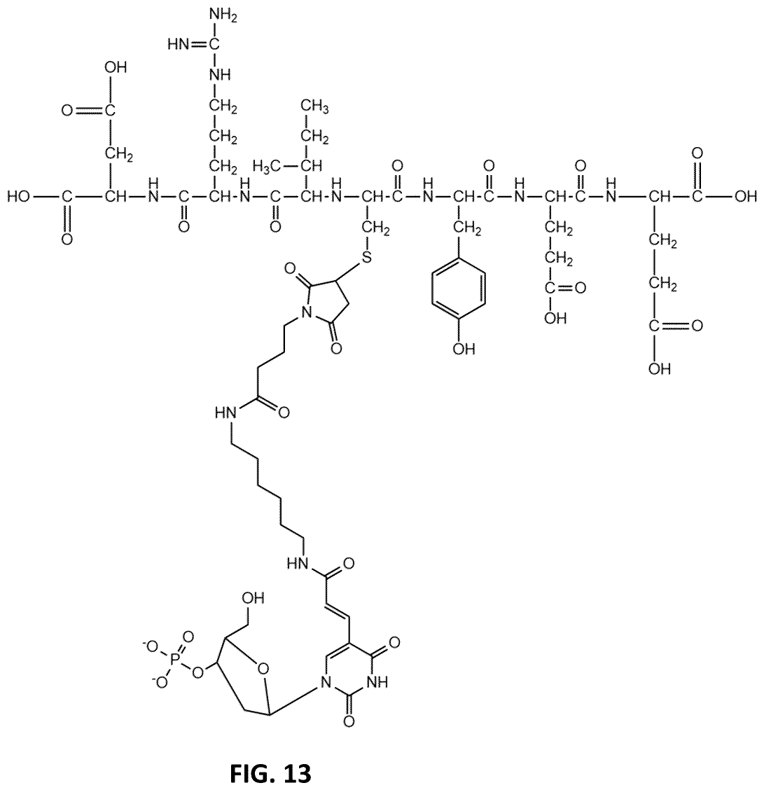

FIG. 13 illustrates the structure of a portion of the conjugation product after the maleimide moiety of the sgRNA reacts with a thiol of a cysteine residue of Cas9. Seen here is a fragment of the Cas9 (N77-R78-I79-(C80)-81Y-82L-83Q) reacted with the maleimide on the guide.

### Example 3: Preparation of Locked Ribonucleoprotein (RNP) and Cleavage of Target DNA by Locked RNP

sgRNA synthesis. sgRNAs (Table 1) were synthesized on Synthego proprietary solid-phase synthesis platform, using CPG as a solid support. 5-Benzylthio-1H-tetrazole (BTT, 0.25 M solution in acetonitrile) was used for coupling, (3-((Dimethylamino-methylidene)amino)-3H-1,2,4-dithiazole-3-thione (DDTT, 0.1 M solution in pyridine) was used for thiolation, dichloroacetic acid (DCA, 3% solution in toluene) used for detritylation. After synthesis, oligonucleotides were subject to a series of deprotection steps, followed by purification by solid phase extraction (SPE). Purified oligonucleotides were analyzed by ESI-MS. All materials for RNA synthesis were obtained from either ChemGenes or Thermo Fisher Scientific, with the exception of amino-modifier C6 dT phosphoramidite (Glen Research, 10-1039-05).

TABLE 1
Sequences of sgRNA

| Target | Protospacer sequence | Locked sgRNA full sequence |
|---|---|---|
| FANCF | GCTGCAGAAGGGATTCCATG | 5′- |
| | SEQ ID NO: 5 | mG*mC*mU*GCAGAAGGGAUUCCAUG |
| | | GAUUUAGAGCUAGAAAUAGCAAGUUA |
| | | AAY**UAAGGCUAGUCCGUUAUCAAC |
| | | UUGAAAAAGUGGCACCGAGUCGGUGC |
| | | UmU*mU*mU-3′ |
| | | SEQ ID NO: 191 |
| RelA | GATCTCCACATAGGGGCCAG | 5′- |
| | SEQ ID NO: 190 | mG*mA*mU*CUCCACAUAGGGGCCAGG |
| | | AUUUAGAGCUAGAAAUAGCAAGUUAA |
| | | AY**UAAGGCUAGUCCGUUAUCAACU |
| | | UGAAAAAGUGGCACCGAGUCGGUGCU |
| | | mU*mU*mU-3′ |
| | | SEQ ID NO: 192 |

Y = dT - C6NH2
** = maleimide

Maleimide derivatization of amino-modified s2RNA. 30 nmol of sgRNA was resuspended in HEPES buffer (60 uL, 0.1 M, pH 8.5). N-γ-maleimidobutyryl-oxysuccinimide ester (GMBS, Thermo, 22309) solution (8.3 mM in DMSO, 240 uL) was added to the sgRNA and incubated at r.t. overnight. Buffer exchange into MES (0.1 M, pH 6.5) was performed using Zeba columns (Thermo, 89883) following the manufacturer's protocol. Identity and purity of the material was evaluated using ESI-MS.

Locked RNP preparation for in vitro assay. 3 nmol of maleimide-derivatized sgRNA was resuspended in MES buffer (150 uL, 0.1 M pH 6.5) and SpCas9 (Aldevron 9212) (50 uL, 1 nmol, 20 uM) was added. This reaction mixture was incubated at r. t. overnight to allow conjugation of the sgRNA and SpCas9 to form a locked ribonucleoprotein (RNP) complex.

Unlocked RNP preparation for in vitro assay. SpCas9 (1 nmol, 20 uM) was added to sgRNA (3 nmol, 20 uM in TE buffer) and incubated at r. t. overnight to allow formation of a non-locked complex.

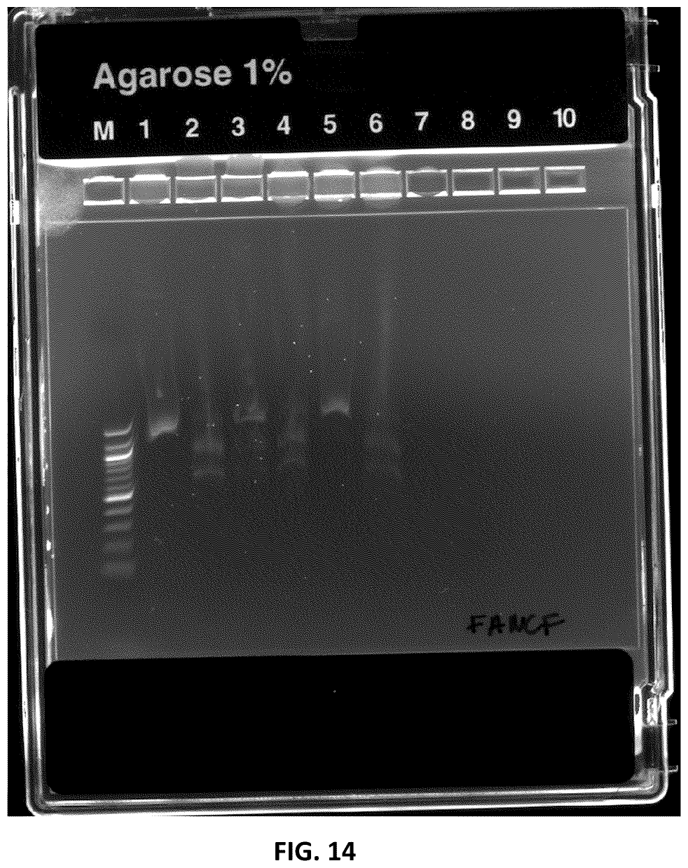

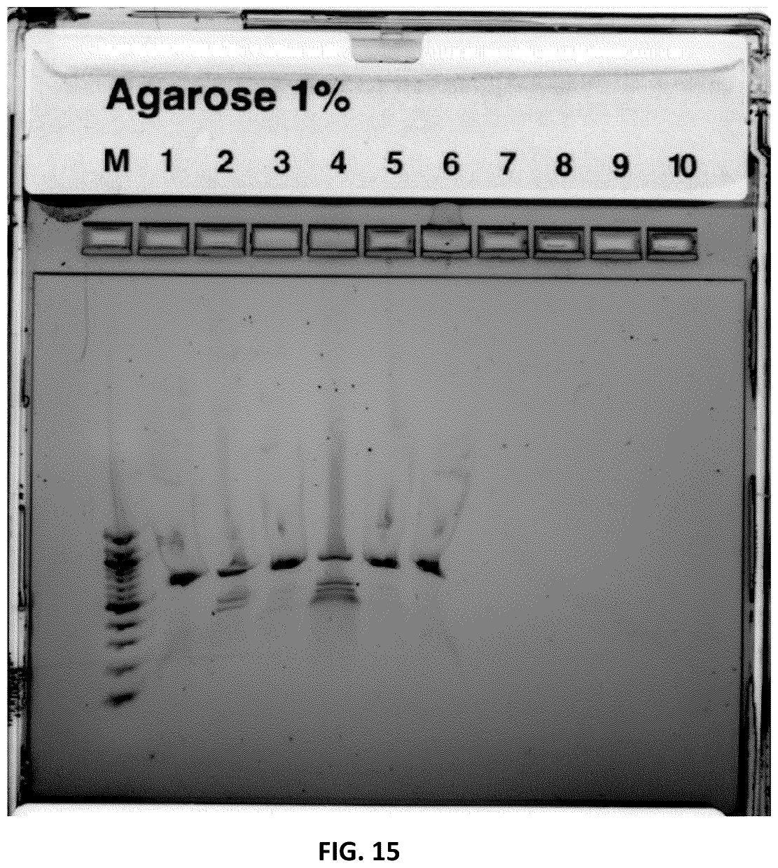

In vitro DNA cleavage assay. gDNA from HEK293T cells at density 1.31E×6 was extracted using DNA QuickExtract (Lucigen, QE09050) and PCR-amplified using primers associated with the oligonucleotide targets (Table 2). PCR products were purified using the ZYMO Research DNA clean and concentrator kit (D4029). In vitro digestion of DNA with SpCas9 was performed using the NEB protocol for the in vitro digestion of DNA (300 nM pre-formed regular or Locked RNP was used). Analysis of the DNA cleavage products was performed using a 1% Agarose EX Gel (Invitrogen, G401001). 20 μL of the reaction mixture or DNA substrate was loaded onto the gel and electrophoresis was run for 10 minutes. Gels were analyzed using BioRad gel imager Chemidoc (FIG. 14 and FIG. 15).

TABLE 2
List of primers used for amplification and sequencing of regions of interest.

| | Primer_F | Primer_R | Primer Seq |
|---|---|---|---|
| FANCF | ATCAGAGAGTCCTCC | GATATTTCCAAAGCGA | GATATTTCCAAAGC |
| | TGGAGATTT | AAGGAAGC | GAAAGGAAGC |
| | SEQ ID NO: 103 | SEQ ID NO: 104 | SEQ ID NO: 104 |
| RelA | GCCCAGACATCCAAA | CGTGGAGAGCAGATT | CCCAGACATCCAAA |
| | CCTGA | CCTCC | CCTGACTC |
| | SEQ ID NO: 193 | SEQ ID NO: 194 | SEQ ID NO: 195 |

Regular RNP Preparation for SDS PAGE and Native PAGE. sgRNA (RelA) (1 nmol, 3 nmol, 4 nmol, or 5 nmol) was resuspended to 20 uM in MES buffer (0.1 M pH 6.5) and then SpCas9 (50 uL, 1 nmol, 20 uM) was added to each sample. The reaction mixture was incubated at r. t. overnight.

Locked RNP Preparation for SDS PAGE and Native PAGE. Maleimide-derivatized sgRNA (RelA) (1 nmol, 3 nmol, 4 nmol, or 5 nmol) was resuspended to 20 uM in MES buffer (0.1 M pH 6.5) and then SpCas9 (50 uL, 1 nmol, 20 uM) was added to each sample. The reaction mixture was incubated at r. t. overnight.

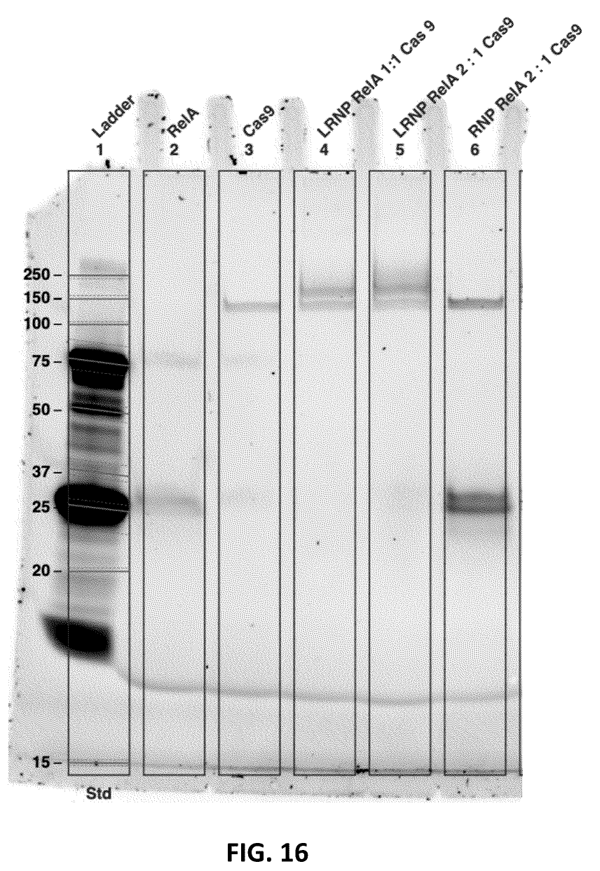

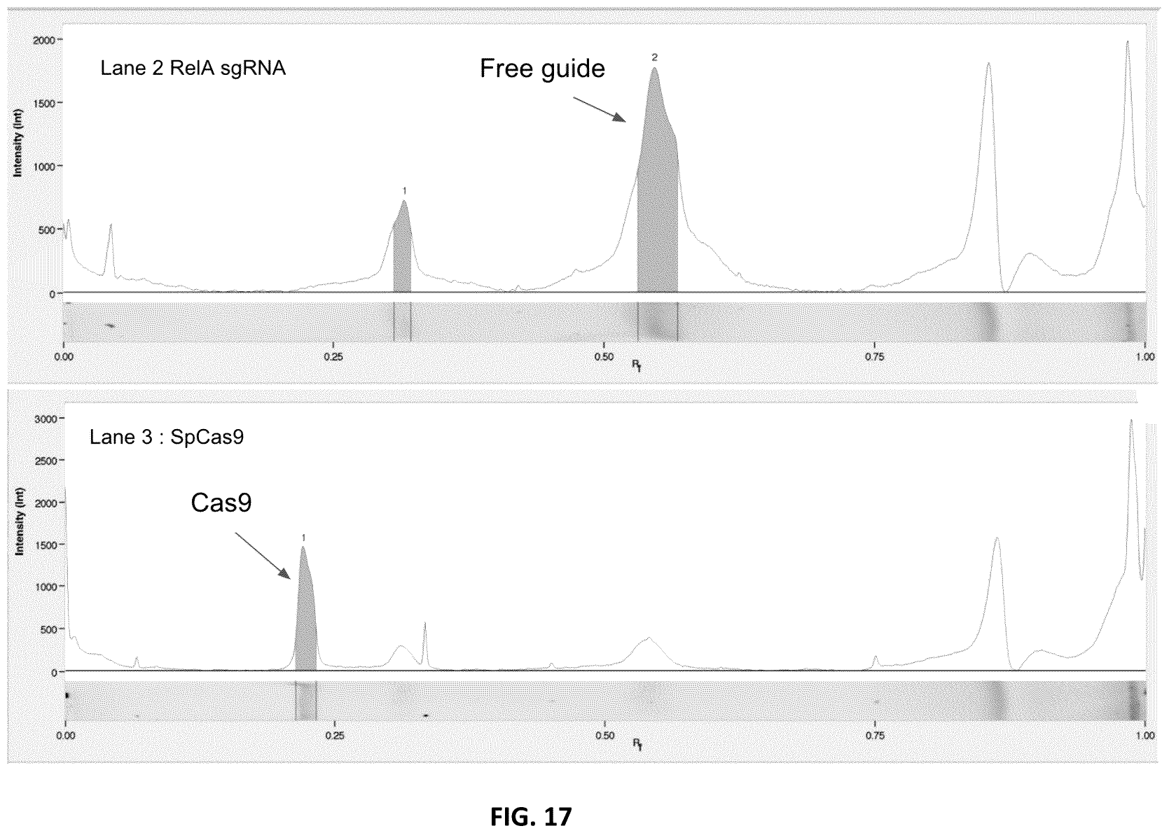

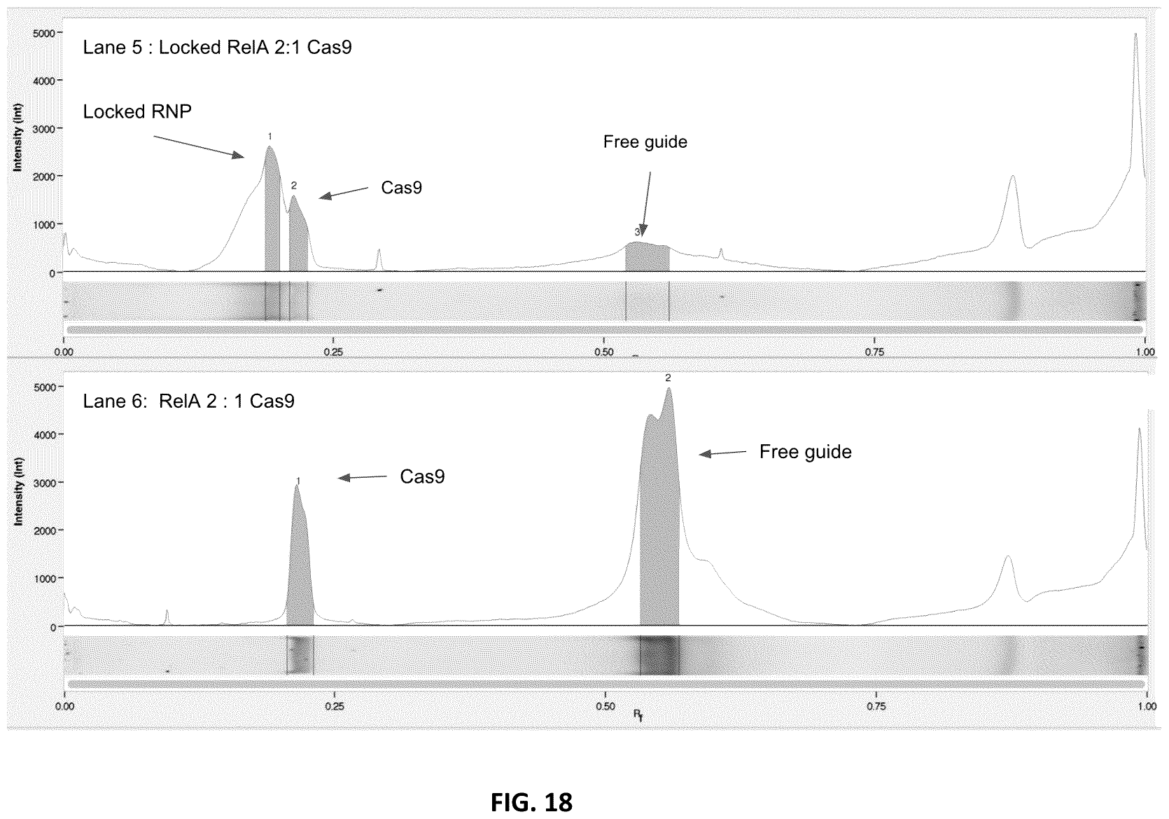

SDS-PAGE gel electrophoresis. In PCR tubes 17 uL nuclease free water, 1 uL RelA RNP sample or 1 uL RelA Locked RNP sample, 3 μL of Invitrogen™ 10× Bolt™ Sample Reducing Agent (B0009) and 7 uL NuPAGE™ LDS Sample Buffer (4×) (NP0007) were combined. Samples were denatured at 90° C. for 5 min, then incubated at 4° C. for 5 minutes. 20 μL of each sample was added onto a Mini-Protean-TGX 4-15% (BioRad, 4561084) along with 2 μL of Precision Plus Protein Dual Color Standards (BioRad, 1610374). Electrophoresis was run at 100 V for 1 hour. The gel was imaged using the BioRad Chemidoc, using stain-free technology followed by SYBR Gold staining (FIG. 16). Image Lab was used for gel analysis and provided the gel lane profiles (FIG. 17 and FIG. 18).

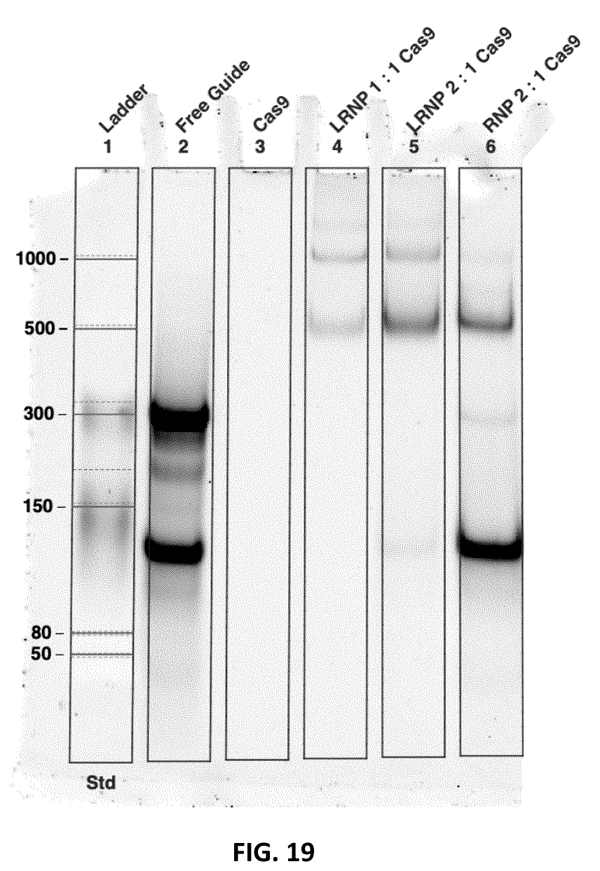

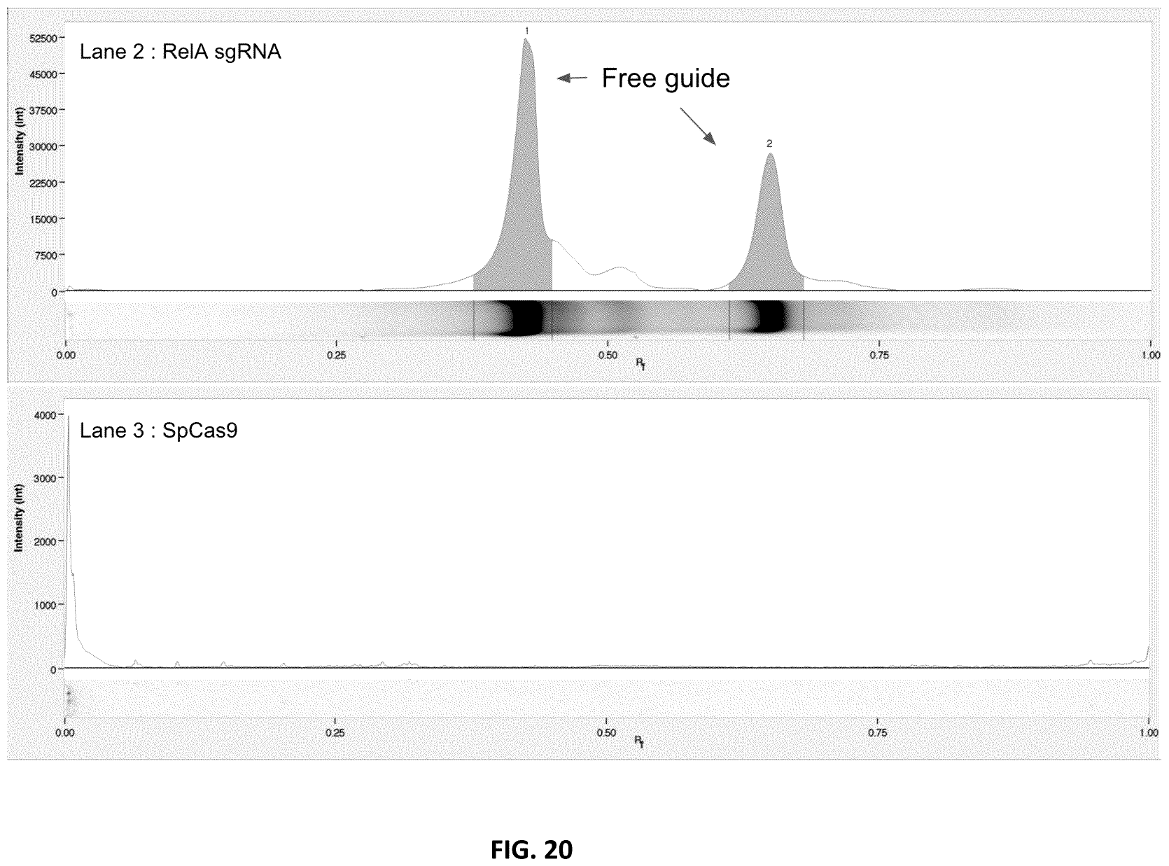

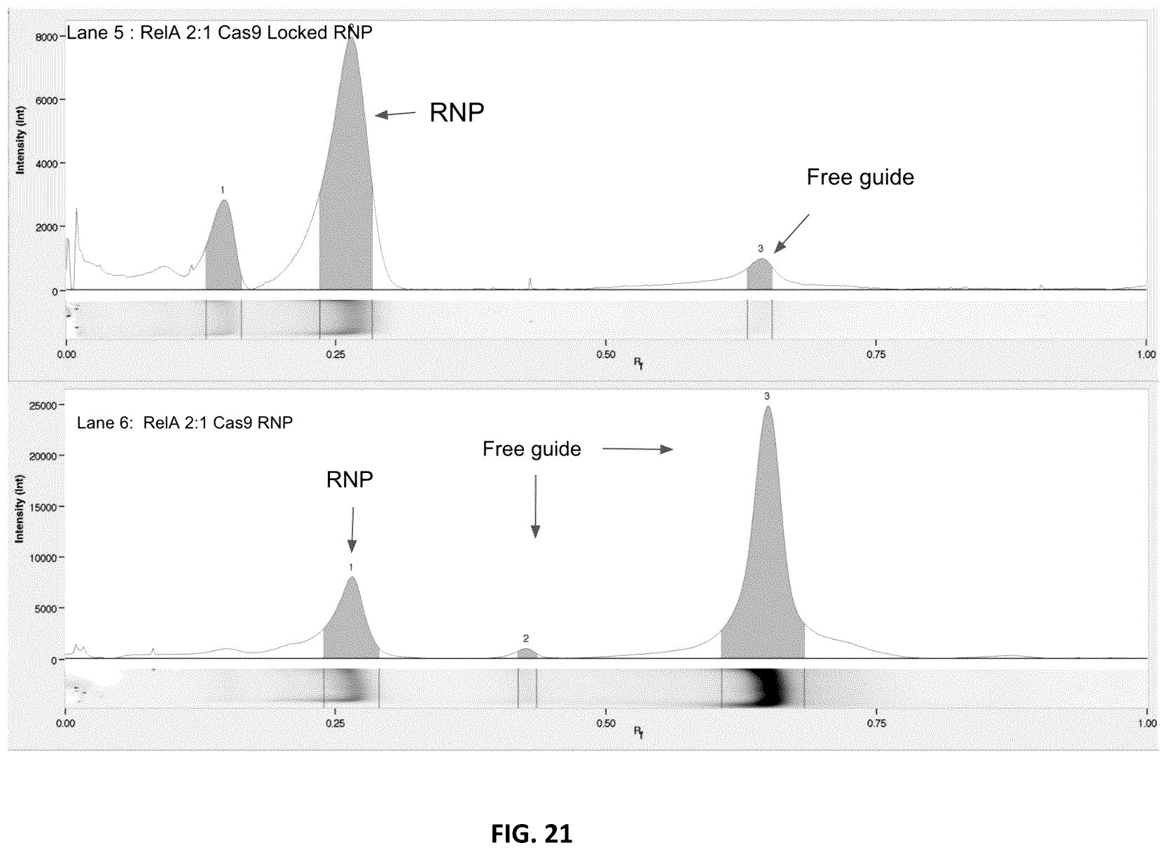

Native PAGE. In PCR tubes, 3 μL of sample was added to 10 uL Ultra Pure distilled RNAse free water and 2.2 uL TBE Sample Buffer 6×(Bioworld, 10570028-1). 12 μL of each sample was loaded onto a 5% TBE Mini-PROTEAN gel, along with Low Range ssRNA Ladder (NEB, N0364S). Electrophoresis was run at 100 V for 1 hour. The gel was imaged using the BioRad Chemidoc, using stain-free technology followed by SYBR Gold staining (FIG. 19). Image Lab was used for gel analysis and provided the gel lane profiles (FIG. 20 and FIG. 21).

Cell culture. HEK293T cells were maintained between passage 5-20 in Dulbecco's Modified Eagle Medium (Gibco) and 10% v/v FBS. Cells were passaged 2-3 times a week at a 1:10 ratio after dissociation with TrypLE (Life Technologies).

Unlocked RNP preparation for cell assay. SpCas9 (1 nmol, 20 uM) was added to sgRNA (3 nmol, 20 uM in Lonza SF buffer with Supplement 1) and incubated at r. t. overnight.

Locked RNP preparation for cell assay. Maleimide-derivatized Locked sgRNA (3 nmol) was resuspended in MES buffer (150 uL, 0.1 M, pH 6.5), and SpCas9 (1 nmol, 20 uM) was added. Reaction mixture was incubated at r. t. overnight. Buffer exchange into the Lonza buffer SF was performed using Zeba columns (Thermo, 89883).

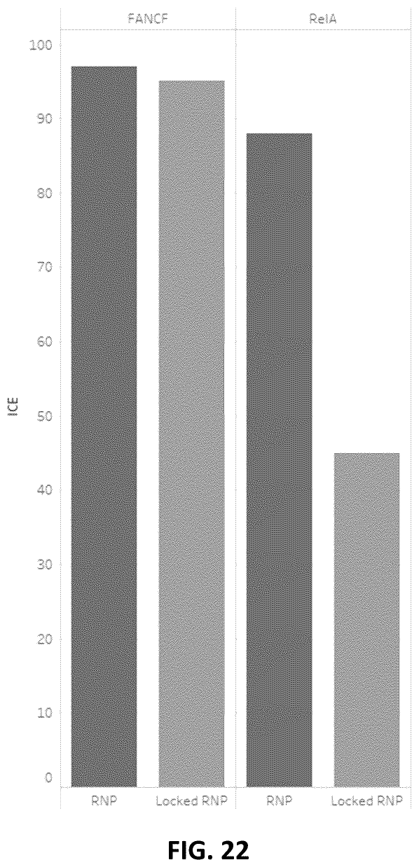

Gene editing analysis. RNP solutions were transfected into cells using the 4D-Nucleofector system (Lonza) in the 20 μL format. HEK293T transfections were conducted in a supplemented Lonza SF buffer using protocol CM-130. Following transfection, cells were recovered in culture media and harvested 24 h post-transfection. gDNA was extracted using DNA QuickExtract (Lucigen, QE09050) following the manufacturer's protocol and regions of interest amplified using primers listed in Table 2. Following Sanger sequencing, the presence of indels was analyzed via ICE (ice.synthego.com) (FIG. 22).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in any combination in practicing the invention. It is intended that the following claims define the scope of the invention and that methods and structures within the scope of these claims and their equivalents be covered thereby.

1. A CRISPR complex comprising a guide RNA (gRNA) conjugated to a CRISPR effector protein via a modified nucleotide within the gRNA, wherein the gRNA is a single guide RNA (sgRNA) comprising a crRNA region comprising a target binding region and a tracrRNA region that comprises the CRISPR effector protein binding region, and wherein the modified nucleotide is within the tracrRNA region.

2. (canceled)

3. The CRISPR complex of claim 1, wherein the gRNA is conjugated to the CRISPR effector protein via the modified nucleotide conjugated to a cysteine residue in the CRISPR effector protein.

4. (canceled)

5. The CRISPR complex of claim 1, wherein the CRISPR effector protein is Cas9.

6. The CRISPR complex of claim 5, wherein the modified nucleotide is conjugated to a Cys80 in the Cas9 enzyme.

7. The CRISPR complex of claim 1, wherein the gRNA or the sgRNA is conjugated to the CRISPR effector protein via the modified nucleotide comprising a maleimide moiety conjugated to a thiol of a cysteine residue in the CRISPR effector protein.

8. The CRISPR complex of claim 1, wherein the modified nucleotide is within 20 angstroms of a cysteine residue of the CRISPR effector protein when the gRNA or sgRNA binds to the CRISPR effector protein.

9. The CRISPR complex of claim 1, wherein the CRISPR effector protein comprises a non-proteogenic amino acid and the gRNA or sgRNA is conjugated to the CRISPR effector protein via the modified nucleotide conjugated to the non-proteogenic amino acid.

10. The CRISPR complex of claim 9, wherein the non-proteogenic amino acid is an azido-containing amino acid.

11. The CRISPR complex of claim 1, wherein the modified nucleotide is at one or more nucleotide positions selected from the group consisting of positions: 22, 23, 24, 25, 31, 37, 44, 49, 45, 50, 56, 59, 63, 64, 66, 71, 72, 77, 78, 80, 84, 90 and 94 of the sgRNA, wherein nucleotide position 1 is at the 5′ end of the target binding region of the crRNA and nucleotide positions of the sgRNA are numbered consecutively from 5′ to 3′ from nucleotide position 1.

12. The CRISPR complex of claim 1, wherein the modified nucleotide is in a stem loop of the tracrRNA region, a bulge of the tracrRNA region, or is between the stem loops of the tracrRNA region.

13. (canceled)

14. (canceled)

15. (canceled)

16. (canceled)

17. The CRISPR complex of claim 1, wherein the modified nucleotide is a modified uracil nucleotide or a modified thymidine nucleotide.

18. The CRISPR complex of claim 1, wherein the modified nucleotide comprises: a modified sugar moiety, a modified base, a maleimide moiety, a N-hydroxysuccinimide (NHS) moiety, a diazirine moiety.

19. (canceled)

20. The CRISPR complex of claim 1, wherein the modified nucleotide comprises 5′-dimethoxytrityl-5-[N-(trifluoroacetylaminohexyl)-3-acrylimido]-2′-deoxyuridine, 3′-[(2-cyanoethyl)-(N,N-diisopropyl)]-phosphoramidite, 5′-dimethoxytrityl-5-[N-(4-maleimidobutyramido)hexyl)-3-acrylimido]-2′-deoxyuridine, 3′-[(2-cyanoethyl)-(N,N-diisopropyl)]-phosphoramidite, or 4-thio-UTP, 5-azido-UTP, 5-bromo-UTP, 8-azido-ATP, 5-APAS-UTP, or 8-N(3)AMP.

21. (canceled)

22. (canceled)

23. (canceled)

24. A single guide (sgRNA) comprising a crRNA region comprising a target binding region and a tracrRNA that comprises a CRISPR effector protein binding region, wherein the tracrRNA region comprises a modified nucleotide that is capable of conjugating with the CRISPR effector protein when the sgRNA binds to the CRISPR effector protein.

25. A sgRNA of claim 24, wherein the modified nucleotide is capable of conjugating with a thiol group of the CRISPR effector protein.

26. The sgRNA of claim 24, wherein the thiol group of the CRISPR effector protein is part of a naturally occurring cysteine residue in the CRISPR effector protein.

27. The sgRNA of claim 26, wherein the cysteine residue is a Cys80 in a Cas9 enzyme.

28. The sgRNA of claim 24, wherein the modified nucleotide is at one or more nucleotide positions selected from the group consisting of positions: 22, 23, 24, 25, 31, 37, 44, 49, 45, 50, 56, 59, 63, 64, 66, 71, 72, 77, 78, 80, 84, 90 and 94 of the sgRNA, wherein nucleotide position 1 is at the 5′ end of the target binding region of the crRNA and nucleotide positions of the sgRNA are numbered consecutively from 5′ to 3′ from nucleotide position 1.

29. A pharmaceutical formulation comprising one or more of the CRISPR complex of claim 1.

30. (canceled)

31. (canceled)

32. A method of editing a target nucleic acid molecule comprising contacting the CRISPR complex of claim 1 with the target nucleic acid molecule.

33. A method of editing a target gene in one or more cells comprising administering the CRISPR complex of claim 1 to the one or more cells comprising the target gene, thereby editing the target gene in the one or more cells.


SEQUENCE LISTING (195 sequences)

SEQ ID NO: 1-3 - skipped (3 entries)

SEQ ID NO: 4 - DNA, 20 bp, synthetic construct
  misc_feature 1..20: note = Synthetic- EMX1
  source 1..20: mol_type = other DNA
gagtccgagcagaagaagaa

SEQ ID NO: 5 - DNA, 20 bp, synthetic construct
  misc_feature 1..20: note = Synthetic- FANCF
  source 1..20: mol_type = other DNA
gctgcagaagggattccatg

SEQ ID NO: 6 - DNA, 20 bp, synthetic construct
  misc_feature 1..20: note = Synthetic- PRGN
  source 1..20: mol_type = other DNA
cagatgcctgctcagtgttg

SEQ ID NO: 7 - DNA, 20 bp, synthetic construct
  misc_feature 1..20: note = Synthetic- VEGFA
  source 1..20: mol_type = other DNA
ggtgagtgagtgtgtgcgtg

SEQ ID NO: 8 - RNA, 100 nt, synthetic construct
  misc_feature 1..100: note = Synthetic- Control sgRNA
  modified_base 1..3: mod_base = OTHER; note = 2'-O-methyl analogs and 3' phosphorothioate internucleotide linkages
  misc_difference 1..20: note = n is a, c, g, or u
  modified_base 98..100: mod_base = OTHER; note = 2'-O-methyl analogs and 3' phosphorothioate internucleotide linkages
  source 1..100: mol_type = other RNA
nnnnnnnnnnnnnnnnnnnngttttagagctagaaatagcaagttaaaataaggctagtccgttatcaacttgaaaaagtggcaccgagtcggtgctttt

SEQ ID NO: 9 - RNA, 104 nt, synthetic construct
  misc_feature 1..104: note = Synthetic- No 2nd sgRNA
  modified_base 1..3: mod_base = OTHER; note = 2'-O-methyl analogs and 3' phosphorothioate internucleotide linkages
  misc_difference 5..24: note = n is a, c, g, or u
  modified_base 102..104: mod_base = OTHER; note = 2'-O-methyl analogs and 3' phosphorothioate internucleotide linkages
  source 1..104: mol_type = other RNA
gaaannnnnnnnnnnnnnnnnnnngttttagagctagaaatagcaagttaaaataaggctagtccgttatcaacttgaaaaagtggcaccgagtcggtgctttt

SEQ ID NO: 10 - RNA, 110 nt, synthetic construct
  misc_feature 1..110: note = Synthetic- 3 bp Stem sgRNA
  modified_base 1..3: mod_base = OTHER; note = 2'-O-methyl analogs and 3' phosphorothioate internucleotide linkages
  misc_difference 11..30: note = n is a, c, g, or u
  modified_base 108..110: mod_base = OTHER; note = 2'-O-methyl analogs and 3' phosphorothioate internucleotide linkages
  source 1..110: mol_type = other RNA
tgagaaatcannnnnnnnnnnnnnnnnnnngttttagagctagaaatagcaagttaaaataaggctagtccgttatcaacttgaaaaagtggcaccgagtcggtgctttt

SEQ ID NO: 11 - RNA, 116 nt, synthetic construct
  misc_feature 1..116: note = Synthetic- 6 bp Stem sgRNA
  modified_base 1..3: mod_base = OTHER; note = 2'-O-methyl analogs and 3' phosphorothioate internucleotide linkages
  misc_difference 17..36: note = n is a, c, g, or u
  modified_base 114..116: mod_base = OTHER; note = 2'-O-methyl analogs and 3' phosphorothioate internucleotide linkages
  source 1..116: mol_type = other RNA
cactgagaaatcagtgnnnnnnnnnnnnnnnnnnnngttttagagctagaaatagcaagttaaaataaggctagtccgttatcaacttgaaaaagtggcaccgagtcggtgctttt

SEQ ID NO: 12 - RNA, 100 nt, synthetic construct; identical to SEQ ID NO: 8
  misc_feature 1..100: note = Synthetic- sgRNA
  modified_base 1..3: mod_base = OTHER; note = 2'-O-methyl analogs and 3' phosphorothioate internucleotide linkages
  misc_difference 1..20: note = n is a, c, g, or u
  modified_base 98..100: mod_base = OTHER; note = 2'-O-methyl analogs and 3' phosphorothioate internucleotide linkages
  source 1..100: mol_type = other RNA

SEQ ID NO: 13 - RNA, 99 nt, synthetic construct
  misc_feature 1..99: note = Synthetic- sgRNA
  modified_base 1..3: mod_base = OTHER; note = 2'-O-methyl analogs and 3' phosphorothioate internucleotide linkages
  misc_difference 1..20: note = n is a, c, g, or u
  modified_base 20^21: mod_base = OTHER; note = Photocleavable bond
  modified_base 97..99: mod_base = OTHER; note = 2'-O-methyl analogs and 3' phosphorothioate internucleotide linkages
  source 1..99: mol_type = other RNA
nnnnnnnnnnnnnnnnnnnnttttagagctagaaatagcaagttaaaataaggctagtccgttatcaacttgaaaaagtggcaccgagtcggtgctttt

SEQ ID NO: 14 - RNA, 99 nt, synthetic construct
  misc_feature 1..99: note = Synthetic- sgRNA
  modified_base 1..3: mod_base = OTHER; note = 2'-O-methyl analogs and 3' phosphorothioate internucleotide linkages
  misc_difference 1..20: note = n is a, c, g, or u
  modified_base 23^24: mod_base = OTHER; note = photocleavable bond
  modified_base 97..99: mod_base = OTHER; note = 2'-O-methyl analogs and 3' phosphorothioate internucleotide linkages
  source 1..99: mol_type = other RNA
nnnnnnnnnnnnnnnnnnnngtttagagctagaaatagcaagttaaaataaggctagtccgttatcaacttgaaaaagtggcaccgagtcggtgctttt

SEQ ID NO: 15 - RNA, 99 nt, synthetic construct
  misc_feature 1..99: note = Synthetic- sgRNA
  modified_base 1..3: mod_base = OTHER; note = 2'-O-methyl analogs and 3' phosphorothioate internucleotide linkages
  misc_difference 1..20: note = n is a, c, g, or u
  modified_base 49^50: mod_base = OTHER; note = photocleavable bond
  modified_base 97..99: mod_base = OTHER; note = 2'-O-methyl analogs and 3' phosphorothioate internucleotide linkages
  source 1..99: mol_type = other RNA
nnnnnnnnnnnnnnnnnnnngttttagagctagaaatagcaagttaaaaaaggctagtccgttatcaacttgaaaaagtggcaccgagtcggtgctttt

SEQ ID NO: 16 - RNA, 99 nt, synthetic construct
  misc_feature 1..99: note = Synthetic- sgRNA
  modified_base 1..3: mod_base = OTHER; note = 2'-O-methyl analogs and 3' phosphorothioate internucleotide linkages
  misc_difference 1..20: note = n is a, c, g, or u
  modified_base 56^57: mod_base = OTHER; note = photocleavable bond
  misc_feature 97..99: note = 2'-O-methyl analogs and 3' phosphorothioate internucleotide linkages
  source 1..99: mol_type = other RNA
nnnnnnnnnnnnnnnnnnnngttttagagctagaaatagcaagttaaaataaggctgtccgttatcaacttgaaaaagtggcaccgagtcggtgctttt

SEQ ID NO: 17 - RNA, 99 nt, synthetic construct
  misc_feature 1..99: note = Synthetic- sgRNA
  modified_base 1..3: mod_base = OTHER; note = 2'-O-methyl analogs and 3' phosphorothioate internucleotide linkages
  misc_difference 1..20: note = n is a, c, g, or u
  modified_base 73^74: mod_base = OTHER; note = photocleavable bond
  modified_base 97..99: mod_base = OTHER; note = 2'-O-methyl analogs and 3' phosphorothioate internucleotide linkages
  source 1..99: mol_type = other RNA
nnnnnnnnnnnnnnnnnnnngttttagagctagaaatagcaagttaaaataaggctagtccgttatcaacttgaaaagtggcaccgagtcggtgctttt

SEQ ID NO: 18 - DNA, 20 bp, synthetic construct
  misc_feature 1..20: note = Synthetic- BUB1B target binding region
  source 1..20: mol_type = other DNA
agtgaagccatgtccctgga

SEQ ID NO: 19 - DNA, 20 bp, synthetic construct
  misc_feature 1..20: note = Synthetic- CAMK1 target binding region
  source 1..20: mol_type = other DNA
tgccaggatcacctccgaga

SEQ ID NO: 20 - DNA, 20 bp, synthetic construct
  misc_feature 1..20: note = Synthetic- PRKAG3 target binding regoin
  source 1..20: mol_type = other DNA
agcaagaaaacagcagctca

SEQ ID NO: 21 - RNA, 20 nt, synthetic construct
  misc_feature 1..20: note = Synthetic- PRKAG3 sg2 target binding region
  source 1..20: mol_type = other RNA
agcaagaaaacagcagctca

SEQ ID NO: 22 - DNA, 20 bp, synthetic construct
  misc_feature 1..20: note = Synthetic- STK3 target binding region
  source 1..20: mol_type = other DNA
tcctgaagatctgattcaac

SEQ ID NO: 23 - DNA, 20 bp, synthetic construct
  misc_feature 1..20: note = Synthetic- STK3 sg2 target binding region
  source 1..20: mol_type = other DNA
aaagcaatacacaaggaatc

SEQ ID NO: 24 - DNA, 20 bp, synthetic construct
  misc_feature 1..20: note = Synthetic- STK3 sg3 target binding region
  source 1..20: mol_type = other DNA
ccataatgcagcaatgtgac

SEQ ID NO: 25 - RNA, 20 nt, synthetic construct
  misc_feature 1..20: note = Synthetic- STK3 sg4 target binding region
  source 1..20: mol_type = other RNA
tttaattgcgacaacttgac

SEQ ID NO: 26 - DNA, 20 bp, synthetic construct
  misc_feature 1..20: note = Synthetic- IRAK4 target binding region
  source 1..20: mol_type = other DNA
gtcctgtctttgtcacagaa

SEQ ID NO: 27 - DNA, 20 bp, synthetic construct
  misc_feature 1..20: note = Synthetic- Chr8q23 target binding region
  source 1..20: mol_type = other DNA
agtctactatgagttttctg

SEQ ID NO: 28 - DNA, 20 bp, synthetic construct
  misc_feature 1..20: note = Synthetic- Chr8q23 sg2 target binding region
  source 1..20: mol_type = other DNA
ttatagttacgatgtttgat

SEQ ID NO: 29 - DNA, 20 bp, synthetic construct
  misc_feature 1..20: note = Synthetic- Chr8q23 sg3 target binding region
  source 1..20: mol_type = other DNA
aagcctcaaattaggagaaa

SEQ ID NO: 30 - DNA, 20 bp, synthetic construct
  misc_feature 1..20: note = Synthetic- DNMT1 target binding region
  source 1..20: mol_type = other DNA
ggagtgagggaaacggcccc

SEQ ID NO: 31 - DNA, 20 bp, synthetic construct
  misc_feature 1..20: note = Synthetic- EMX1 target binding region
  source 1..20: mol_type = other DNA
gagtccgagcagaagaagaa

SEQ ID NO: 32 - DNA, 20 bp, synthetic construct
  misc_feature 1..20: note = Synthetic- FANCF target binding region
  source 1..20: mol_type = other DNA
gctgcagaagggattccatg

SEQ ID NO: 33 - DNA, 20 bp, synthetic construct
  misc_feature 1..20: note = Synthetic- GRK1 target binding region
  source 1..20: mol_type = other DNA
gccgtcaaagctgcctcggg

SEQ ID NO: 34 - DNA, 20 bp, synthetic construct
  misc_feature 1..20: note = Synthetic- PRGN target binding region
  source 1..20: mol_type = other DNA
cagatgcctgctcagtgttg

SEQ ID NO: 35 - DNA, 20 bp, synthetic construct
  misc_feature 1..20: note = Synthetic- VEGFA target binding region
  source 1..20: mol_type = other DNA
ggtgagtgagtgtgtgcgtg

SEQ ID NO: 36 - DNA, 100 bp, synthetic construct
  misc_feature 1..100: note = Synthetic- on-target sgRNA
  source 1..100: mol_type = other DNA
ggagtgagggaaacggccccgttttagagctagaaatagcaagttaaaataaggctagtccgttatcaacttgaaaaagtggcaccgagtcggtgctttt

SEQ ID NO: 37 - DNA, 98 bp, synthetic construct
  misc_feature 1..98: note = Synthetic- on-target CRISPR OFF
  modified_base 57: mod_base = OTHER; note = (3-(4,4'-Dimethoxytrityl)-1-(2-nitrophenyl)-propan- 1-yl-[(2-cyano ethyl)-(N,N-diisopropyl)]-phosphoramidite) linker
  modified_base 74: mod_base = OTHER; note = (3-(4,4'-Dimethoxytrityl)-1-(2-nitrophenyl)-propan- 1-yl-[(2-cyano ethyl)-(N,N-diisopropyl)]-phosphoramidite) linker
  source 1..98: mol_type = other DNA
ggagtgagggaaacggccccgttttagagctagaaatagcaagttaaaataaggctgtccgttatcaacttgaaaagtggcaccgagtcggtgctttt

SEQ ID NO: 38 - DNA, 20 bp, synthetic construct
  misc_feature 1..20: note = Synthetic- Off-target 1
  source 1..20: mol_type = other DNA
ggagggagggaaacagcccc

SEQ ID NO: 39 - DNA, 100 bp, synthetic construct
  misc_feature 1..100: note = Synthetic- On-target sgRNA
  source 1..100: mol_type = other DNA
gctgcagaagggattccatggttttagagctagaaatagcaagttaaaataaggctagtccgttatcaacttgaaaaagtggcaccgagtcggtgctttt

SEQ ID NO: 40 - DNA, 98 bp, synthetic construct
  misc_feature 1..98: note = Synthetic- On-target CRISPR OFF
  modified_base 57: mod_base = OTHER; note = (3-(4,4'-Dimethoxytrityl)-1-(2-nitrophenyl)-propan- 1-yl-[(2-cyano ethyl)-(N,N-diisopropyl)]-phosphoramidite) linker
  modified_base 74: mod_base = OTHER; note = (3-(4,4'-Dimethoxytrityl)-1-(2-nitrophenyl)-propan- 1-yl-[(2-cyano ethyl)-(N,N-diisopropyl)]-phosphoramidite) linker
  source 1..98: mol_type = other DNA
gctgcagaagggattccatggttttagagctagaaatagcaagttaaaataaggctgtccgttatcaacttgaaaagtggcaccgagtcggtgctttt

SEQ ID NO: 41 - DNA, 20 bp, synthetic construct
  misc_feature 1..20: note = Synthetic- Off-target 2
  source 1..20: mol_type = other DNA
gctgcagaagggattccaag

SEQ ID NO: 42 - DNA, 100 bp, synthetic construct
  misc_feature 1..100: note = Synthetic- On target sgRNA
  source 1..100: mol_type = other DNA
ggtgagtgagtgtgtgcgtggttttagagctagaaatagcaagttaaaataaggctagtccgttatcaacttgaaaaagtggcaccgagtcggtgctttt

SEQ ID NO: 43 - DNA, 98 bp, synthetic construct
  misc_feature 1..98: note = Synthetic- On-target CRISPR OFF
  modified_base 57: mod_base = OTHER; note = (3-(4,4'-Dimethoxytrityl)-1-(2-nitrophenyl)-propan- 1-yl-[(2-cyano ethyl)-(N,N-diisopropyl)]-phosphoramidite) linker
  modified_base 74: mod_base = OTHER; note = (3-(4,4'-Dimethoxytrityl)-1-(2-nitrophenyl)-propan- 1-yl-[(2-cyano ethyl)-(N,N-diisopropyl)]-phosphoramidite) linker
  source 1..98: mol_type = other DNA
ggtgagtgagtgtgtgcgtggttttagagctagaaatagcaagttaaaataaggctgtccgttatcaacttgaaaagtggcaccgagtcggtgctttt

SEQ ID NO: 44 - DNA, 20 bp, synthetic construct
  misc_feature 1..20: note = Synthetic- Off-target 3
  source 1..20: mol_type = other DNA
gctgagtgagtgtatgcgtg

SEQ ID NO: 45 - RNA, 20 nt, synthetic construct
  misc_feature 1..20: note = Synthetic- AAVS target binding region
  source 1..20: mol_type = other RNA
ggggccactagggacaggat

SEQ ID NO: 46 - RNA, 20 nt, synthetic construct
  misc_feature 1..20: note = Synthetic- BUB1B target binding region
  source 1..20: mol_type = other RNA
agtgaagccatgtccctgga

SEQ ID NO: 47 - RNA, 20 nt, synthetic construct
  misc_feature 1..20: note = Synthetic- CAMK1_sg1 target binding region
  source 1..20: mol_type = other RNA
tgccaggatcacctccgaga

SEQ ID NO: 48 - RNA, 20 nt, synthetic construct
  misc_feature 1..20: note = Synthetic- CAMK1_sg2 target binding region
  source 1..20: mol_type = other RNA
gcgtcctcttatcttctgcc

SEQ ID NO: 49 - RNA, 20 nt, synthetic construct
  misc_feature 1..20: note = Synthetic- CEL target binding region
  source 1..20: mol_type = other RNA
aaccagttgcaggcgcccca

SEQ ID NO: 50 - RNA, 20 nt, synthetic construct
  misc_feature 1..20: note = Synthetic- Chr8q23 sg1
  source 1..20: mol_type = other RNA
ttatagttacgatgtttgat

SEQ ID NO: 51 - RNA, 20 nt, synthetic construct
  misc_feature 1..20: note = Synthetic- CXCR4 target binding region
  source 1..20: mol_type = other RNA
gataactacaccgaggaaat

SEQ ID NO: 52 - RNA, 20 nt, synthetic construct
  misc_feature 1..20: note = Synthetic- DNMT1 target binding region
  source 1..20: mol_type = other RNA
ggagtgagggaaacggcccc

SEQ ID NO: 53 - RNA, 20 nt, synthetic construct
  misc_feature 1..20: note = Synthetic- EMX1 target binding region
  source 1..20: mol_type = other RNA
gagtccgagcagaagaagaa

SEQ ID NO: 54 - RNA, 20 nt, synthetic construct
  misc_feature 1..20: note = Synthetic- FAM163A target binding region
  source 1..20: mol_type = other RNA
ctgcagggctcgctggtgag

SEQ ID NO: 55 - RNA, 20 nt, synthetic construct
  misc_feature 1..20: note = Synthetic- FANCF target binding region
  source 1..20: mol_type = other RNA
gctgcagaagggattccatg

SEQ ID NO: 56 - RNA, 20 nt, synthetic construct
  misc_feature 1..20: note = Synthetic- GAA target binding region
  source 1..20: mol_type = other RNA
aggagccggtgggagcaggg

SEQ ID NO: 57 - RNA, 20 nt, synthetic construct
  misc_feature 1..20: note = Synthetic- GRK1 target binding region
  source 1..20: mol_type = other RNA
gccgtcaaagctgcctcggg

SEQ ID NO: 58 - RNA, 20 nt, synthetic construct
  misc_feature 1..20: note = Synthetic- ITGA7 target binding region
  source 1..20: mol_type = other RNA
ggtgctggagggcgaggctg

SEQ ID NO: 59 - RNA, 20 nt, synthetic construct
  misc_feature 1..20: note = Synthetic- IRAK4 target binding region
  source 1..20: mol_type = other RNA
gtcctgtctttgtcacagaa

SEQ ID NO: 60 - RNA, 20 nt, synthetic construct
  misc_feature 1..20: note = Synthetic- MAPRE1 target binding region
  source 1..20: mol_type = other RNA
ttctctgcagataattcctg

SEQ ID NO: 61 - RNA, 20 nt, synthetic construct
  misc_feature 1..20: note = Synthetic- MIP target binding region
  source 1..20: mol_type = other RNA
gctggggtcctcactgcgct

SEQ ID NO: 62 - RNA, 20 nt, synthetic construct
  misc_feature 1..20: note = Synthetic- OMP target binding region
  source 1..20: mol_type = other RNA
gaactgtagccgctgctgct

SEQ ID NO: 63 - RNA, 20 nt, synthetic construct
  misc_feature 1..20: note = Synthetic- OPN1SW target binding region
  source 1..20: mol_type = other RNA
acaggggcaatgtggtactg

SEQ ID NO: 64 - RNA, 20 nt, synthetic construct
  misc_feature 1..20: note = Synthetic- PRGN target binding region
  source 1..20: mol_type = other RNA
cagatgcctgctcagtgttg

SEQ ID NO: 65 - RNA, 20 nt, synthetic construct
  misc_feature 1..20: note = Synthetic- PRKAG3 target binding region
  source 1..20: mol_type = other RNA
agcaagaaaacagcagctca

SEQ ID NO: 66 - RNA, 20 nt, synthetic construct
  misc_feature 1..20: note = Sythetic- STK3 sg1
  source 1..20: mol_type = other RNA
aaagcaatacacaaggaatc

SEQ ID NO: 67 - RNA, 20 nt, synthetic construct
  misc_feature 1..20: note = Synthetic- STK3 sg2
  source 1..20: mol_type = other RNA
ccataatgcagcaatgtgac

SEQ ID NO: 68 - RNA, 20 nt, synthetic construct
  misc_feature 1..20: note = Synthetic- VEGFA target binding region
  source 1..20: mol_type = other RNA
ggtgagtgagtgtgtgcgtg

SEQ ID NO: 69 - DNA, 20 bp, synthetic construct
  misc_feature 1..20: note = Synthetic- AAVS F
  source 1..20: mol_type = other DNA
gcccctatgtccacttcagg

SEQ ID NO: 70 - DNA, 20 bp, synthetic construct
  misc_feature 1..20: note = Synthetic- AAVS R
  source 1..20: mol_type = other DNA
ctcaggttctgggagagggt

SEQ ID NO: 71 - DNA, 25 bp, synthetic construct
  misc_feature 1..25: note = Synthetic- AAVS Primer seq
  source 1..25: mol_type = other DNA
ctccatcgtaagcaaaccttagagg

SEQ ID NO: 72 - DNA, 20 bp, synthetic construct
  misc_feature 1..20: note = Synthetic- BUB1B F
  source 1..20: mol_type = other DNA
agaaatcctcccacttcggc

SEQ ID NO: 73 - DNA, 20 bp, synthetic construct
  misc_feature 1..20: note = Synthetic- BUB1B R
  source 1..20: mol_type = other DNA
gcagattcttgtgccagtgc

SEQ ID NO: 74 - DNA, 30 bp, synthetic construct
  misc_feature 1..30: note = Synthetic- BUB1B primer seq
  source 1..30: mol_type = other DNA
cagctaacaaagaagcttaggcatataata

SEQ ID NO: 75 - DNA, 20 bp, synthetic construct
  misc_feature 1..20: note = Synthetic- CAMK1_sg1F
  source 1..20: mol_type = other DNA
acaaccctgccaagtggaaa

SEQ ID NO: 76 - DNA, 20 bp, synthetic construct
  misc_feature 1..20: note = Synthetic- CAMK1_sg1R
  source 1..20: mol_type = other DNA
actaggggagggtcatccac

SEQ ID NO: 77 - DNA, 30 bp, synthetic construct
  misc_feature 1..30: note = Synthetic- CAMK1_sg1 primer seq
  source 1..30: mol_type = other DNA
cattttataaaggggcaatttaaggcttag

SEQ ID NO: 78 - DNA, 20 bp, synthetic construct
  misc_feature 1..20: note = Synthetic- CAMK1_sg2F
  source 1..20: mol_type = other DNA
acaaccctgccaagtggaaa

SEQ ID NO: 79 - DNA, 20 bp, synthetic construct
  misc_feature 1..20: note = Synthetic- CAMK1_sg2R
  source 1..20: mol_type = other DNA
actaggggagggtcatccac

SEQ ID NO: 80 - DNA, 30 bp, synthetic construct; identical to SEQ ID NO: 77
  misc_feature 1..30: note = Synthetic- CAMK1_sg2 primer seq
  source 1..30: mol_type = other DNA

SEQ ID NO: 81 - DNA, 20 bp, synthetic construct
  misc_feature 1..20: note = Synthetic- CEL F
  source 1..20: mol_type = other DNA
ctgagggtgtagaggggagg

SEQ ID NO: 82 - DNA, 20 bp, synthetic construct
  misc_feature 1..20: note = Synthetic- CEL R
  source 1..20: mol_type = other DNA
gttctacctggcacctgtcc

SEQ ID NO: 83 - DNA, 23 bp, synthetic construct
  misc_feature 1..23: note = Synthetic- CEL primer seq
  source 1..23: mol_type = other DNA
cctgagagctcatgaacaagcat

SEQ ID NO: 84 - DNA, 22 bp, synthetic construct
  misc_feature 1..22: note = Synthetic- Chr8q23_sg1 F
  source 1..22: mol_type = other DNA
ctcgtcaaaacaagggtaagca

SEQ ID NO: 85 - DNA, 20 bp, synthetic construct
  misc_feature 1..20: note = Synthetic- Chr8q23_sg1 R
  source 1..20: mol_type = other DNA
gtttgagttgaccaaacgca

SEQ ID NO: 86 - DNA, 30 bp, synthetic construct
  misc_feature 1..30: note = Synthetic- Chr8q23_sg1 primer seq
  source 1..30: mol_type = other DNA
caagggtaagcaaagaaataaaatctcttc

SEQ ID NO: 87 - DNA, 20 bp, synthetic construct
  misc_feature 1..20: note = Synthetic- Chr8q23_sg2 F
  source 1..20: mol_type = other DNA
acctgtcacattgctgcatt

SEQ ID NO: 88 - DNA, 20 bp, synthetic construct
  misc_feature 1..20: note = Synthetic- Chr8q23_sg2 R
  source 1..20: mol_type = other DNA
gtttgagttgaccaaacgca

SEQ ID NO: 89 - DNA, 30 bp, synthetic construct
  misc_feature 1..30: note = Synthetic- Chr8q23_sg2 primer seq
  source 1..30: mol_type = other DNA
ttgattatttcctgaagatctgattcaaca

SEQ ID NO: 90 - DNA, 20 bp, synthetic construct
  misc_feature 1..20: note = Synthetic- CXCR4 F
  source 1..20: mol_type = other DNA
ttgtgcccttagcccactac

SEQ ID NO: 91 - DNA, 20 bp, synthetic construct
  misc_feature 1..20: note = Synthetic- CXCR4 R
  source 1..20: mol_type = other DNA
ccagaagggaagcgtgatga

SEQ ID NO: 92 - DNA, 27 bp, synthetic construct
  misc_feature 1..27: note = Synthetic- CXCR4 primer seq
  source 1..27: mol_type = other DNA
gtacttgtccgtcatgcttctcagttt

SEQ ID NO: 93 - DNA, 24 bp, synthetic construct
  misc_feature 1..24: note = Synthetic- DNMT1 F
  source 1..24: mol_type = other DNA
gatcaagctttgtatgttggccaa

SEQ ID NO: 94 - DNA, 24 bp, synthetic construct
  misc_feature 1..24: note = Synthetic- DNMT1 R
  source 1..24: mol_type = other DNA
aatccagaatgcacaaagtactgc

SEQ ID NO: 95 - DNA, 24 bp, synthetic construct
  misc_feature 1..24: note = Synthetic- DNMT1 primer seq
  source 1..24: mol_type = other DNA
gatcaagctttgtatgttggccaa

SEQ ID NO: 96 - DNA, 22 bp, synthetic construct
  misc_feature 1..22: note = Synthetic- EMX1 F
  source 1..22: mol_type = other DNA
cagctctgtgaccctttgtttg

SEQ ID NO: 97 - DNA, 22 bp, synthetic construct
  misc_feature 1..22: note = Synthetic- EMX1 R
  source 1..22: mol_type = other DNA
actaaactacagtggtgcctgg

SEQ ID NO: 98 - DNA, 22 bp, synthetic construct
  misc_feature 1..22: note = Synthetic- EMX1 primer seq
  source 1..22: mol_type = other DNA
cagctctgtgaccctttgtttg

SEQ ID NO: 99 - DNA, 20 bp, synthetic construct
  misc_feature 1..20: note = Synthetic- FAM163A F
  source 1..20: mol_type = other DNA
gagtggtgggaggggaaaag

SEQ ID NO: 100 - DNA, 20 bp, synthetic construct
  misc_feature 1..20: note = Synthetic- FAM163A R
  source 1..20: mol_type = other DNA
catgtcagccgtccgtatgt

SEQ ID NO: 101 - DNA, 25 bp, synthetic construct
  misc_feature 1..25: note = Synthetic- FAM163A primer seq
  source 1..25: mol_type = other DNA
cttgcaaagctgggattagaaactt

SEQ ID NO: 102 - DNA, 24 bp, synthetic construct
  misc_feature 1..24: note = Synthetic- FANCF F
  source 1..24: mol_type = other DNA
gatatttccaaagcgaaaggaagc

SEQ ID NO: 103 - DNA, 24 bp, synthetic construct
  misc_feature 1..24: note = Synthetic- FANCF R
  source 1..24: mol_type = other DNA
atcagagagtcctcctggagattt

SEQ ID NO: 104 - DNA, 24 bp, synthetic construct
  misc_feature 1..24: note = Synthetic- FANCF primer seq
  source 1..24: mol_type = other DNA
gatatttccaaagcgaaaggaagc

SEQ ID NO: 105 - DNA, 20 bp, synthetic construct
  misc_feature 1..20: note = Synthetic- GAA F
  source 1..20: mol_type = other DNA
ggtgagtctcctccaggact

SEQ ID NO: 106 - DNA, 20 bp, synthetic construct
  misc_feature 1..20: note = Synthetic- GAA R
  source 1..20: mol_type = other DNA
cagactgtgcaagtgctctg

SEQ ID NO: 107 - DNA, 22 bp, synthetic construct
  misc_feature 1..22: note = Synthetic- GAA primer seq
  source 1..22: mol_type = other DNA
cttttctcgcccttccttctgg

SEQ ID NO: 108 - DNA, 21 bp, synthetic construct
  misc_feature 1..21: note = Synthetic- GRK1 F
  source 1..21: mol_type = other DNA
gtctctctcgtccagcaaggg

SEQ ID NO: 109 - DNA, 22 bp, synthetic construct
  misc_feature 1..22: note = Synthetic- GRK1 R
  source 1..22: mol_type = other DNA
atgtctttccagagctccaggg

SEQ ID NO: 110 - DNA, 21 bp, synthetic construct
  misc_feature 1..21: note = Synthetic- GRK1 primer seq
  source 1..21: mol_type = other DNA
gtctctctcgtccagcaaggg

SEQ ID NO: 111 - DNA, 20 bp, synthetic construct
  misc_feature 1..20: note = Synthetic- ITGA7 F
  source 1..20: mol_type = other DNA
ggttgtcgccaaaccttcac

SEQ ID NO: 112 - DNA, 20 bp, synthetic construct
  misc_feature 1..20: note = Synthetic- ITGA7 R
  source 1..20: mol_type = other DNA
gggattggggagtcaagagc

SEQ ID NO: 113 - DNA, 30 bp, synthetic construct
  misc_feature 1..30: note = Synthetic- ITGA7 primer seq
  source 1..30: mol_type = other DNA
gagtcaagagcacaagaaacatgagaacat

SEQ ID NO: 114 - DNA, 21 bp, synthetic construct
  misc_feature 1..21: note = Synthetic- IRAK4 F
  source 1..21: mol_type = other DNA
gcttcttgtgtgtgctgtgag

SEQ ID NO: 115 - DNA, 20 bp, synthetic construct
  misc_feature 1..20: note = Synthetic- IRAK4 R
  source 1..20: mol_type = other DNA
gcctgtgattgctgcacaaa

SEQ ID NO: 116 - DNA, 30 bp, synthetic construct
  misc_feature 1..30: note = Synthetic- IRAK4 primer seq
  source 1..30: mol_type = other DNA
caagtttctagtttaactttttcacaacca

SEQ ID NO: 117 - DNA, 22 bp, synthetic construct
  misc_feature 1..22: note = Synthetic- MAPRE1 F
  source 1..22: mol_type = other DNA
ggtactcttgaaggcaaactgc

SEQ ID NO: 118 - DNA, 23 bp, synthetic construct
  misc_feature 1..23: note = Synthetic- MAPRE1 R
  source 1..23: mol_type = other DNA
cgctgaatgaatatctggaacgc

SEQ ID NO: 119 - DNA, 30 bp, synthetic construct
  misc_feature 1..30: note = Synthetic- MAPRE1 primer seq
  source 1..30: mol_type = other DNA
actgcatgaaacttgctttataaatttagg

SEQ ID NO: 120 - DNA, 20 bp, synthetic construct
  misc_feature 1..20: note = Synthetic- MIP F
  source 1..20: mol_type = other DNA
tcagccaaccattaccgtgt

SEQ ID NO: 121 - DNA, 20 bp, synthetic construct
  misc_feature 1..20: note = Synthetic- MIP1R
  source 1..20: mol_type = other DNA
taaaggggactgtccaccca

SEQ ID NO: 122 - DNA, 26 bp, synthetic construct
  misc_feature 1..26: note = Synthetic- MIP primer seq
  source 1..26: mol_type = other DNA
cattaccgtgttgagtgctaggtttc

SEQ ID NO: 123 - DNA, 20 bp, synthetic construct
  misc_feature 1..20: note = Synthetic- OMP F
  source 1..20: mol_type = other DNA
ttgagaactgagtggggctg

SEQ ID NO: 124 - DNA, 20 bp, synthetic construct
  misc_feature 1..20: note = Synthetic- OMP R
  source 1..20: mol_type = other DNA
gcgtgtcatgaggttggtga

SEQ ID NO: 125 - DNA, 20 bp, synthetic construct
  misc_feature 1..20: note = Synthetic- OMP primer seq
  source 1..20: mol_type = other DNA
ttgagaactgagtggggctg

SEQ ID NO: 126 - DNA, 20 bp, synthetic construct
  misc_feature 1..20: note = Synthetic- OPN1SW F
  source 1..20: mol_type = other DNA
cccctaacccctttttcccc

SEQ ID NO: 127 - DNA, 20 bp, synthetic construct
  misc_feature 1..20: note = Synthetic- OPN1SW R
  source 1..20: mol_type = other DNA
gttttgtggggtgggaggat

SEQ ID NO: 128 - DNA, 25 bp, synthetic construct
  misc_feature 1..25: note = Synthetic- OPN1SW primer seq
  source 1..25: mol_type = other DNA
ctaacccctttttcccctgcagtac

SEQ ID NO: 129 - DNA, 20 bp, synthetic construct
  misc_feature 1..20: note = Synthetic- PRGN F
  source 1..20: mol_type = other DNA
tgagctgggtggccttaaca

SEQ ID NO: 130 - DNA, 21 bp, synthetic construct
  misc_feature 1..21: note = Synthetic- PRGN R
  source 1..21: mol_type = other DNA
cattggcagggcccttttatc

SEQ ID NO: 131 - DNA, 20 bp, synthetic construct
  misc_feature 1..20: note = Synthetic- PRGN primer seq
  source 1..20: mol_type = other DNA
ccagatggtcagttctgccc

SEQ ID NO: 132 - DNA, 20 bp, synthetic construct
  misc_feature 1..20: note = Synthetic- PRKAG3_sg1 F
  source 1..20: mol_type = other DNA
atgtagggagactgaggcca

SEQ ID NO: 133 - DNA, 20 bp, synthetic construct
  misc_feature 1..20: note = Synthetic- PRKAG3_sg1 R
  source 1..20: mol_type = other DNA
gcccattggaagcttgcaaa

SEQ ID NO: 134 - DNA, 25 bp, synthetic construct
  misc_feature 1..25: note = Synthetic- PRKAG3_sg1 primer seq
  source 1..25: mol_type = other DNA
ttgggtccaactctgtgttatggag

SEQ ID NO: 135 - DNA, 20 bp, synthetic construct
  misc_feature 1..20: note = Synthetic- STK3_sg1 F
  source 1..20: mol_type = other DNA
acggcaaaaccctgtctcaa

SEQ ID NO: 136 - DNA, 25 bp, synthetic construct
  misc_feature 1..25: note = Synthetic- STK3_sg1 R
  source 1..25: mol_type = other DNA
tccacagaaaactcatagtagactt

SEQ ID NO: 137 - DNA, 30 bp, synthetic construct
  misc_feature 1..30: note = Synthetic- STK3_sg1 primer seq
  source 1..30: mol_type = other DNA
aaacaagggtaagcaaagaaataaaatctc

SEQ ID NO: 138 - DNA, 20 bp, synthetic construct
  misc_feature 1..20: note = Synthetic- STK3_sg2 F
  source 1..20: mol_type = other DNA
aagccatcctcatctgcctt

SEQ ID NO: 139 - DNA, 21 bp, synthetic construct
  misc_feature 1..21: note = Synthetic- STK3_sg2 R
  source 1..21: mol_type = other DNA
acacaaggaatccggtcaagt

SEQ ID NO: 140 - DNA, 30 bp, synthetic construct
  misc_feature 1..30: note = Synthetic- STK3_sg2 primer seq
  source 1..30: mol_type = other DNA
ggagaaacccatctctactaaaaatacaaa

SEQ ID NO: 141 - DNA, 24 bp, synthetic construct
  misc_feature 1..24: note = Synthetic- VEGFA F
  source 1..24: mol_type = other DNA
gaagcaactccagtcccaaatatg

SEQ ID NO: 142 - DNA, 24 bp, synthetic construct
  misc_feature 1..24: note = Synthetic- VEGFA R
  source 1..24: mol_type = other DNA
gttcacagcctgaaaattacccat

SEQ ID NO: 143 - DNA, 24 bp, synthetic construct
  misc_feature 1..24: note = Synthetic- VEGFA primer seq
  source 1..24: mol_type = other DNA
gaagcaactccagtcccaaatatg

SEQ ID NO: 144 - RNA, 20 nt, synthetic construct
  misc_feature 1..20: note = Synthetic- Chr8q23_sg2
  source 1..20: mol_type = other RNA
agtctactatgagttttctg

SEQ ID NO: 145 - DNA, 20 bp, synthetic construct
  misc_feature 1..20: note = Synthetic- MIP_OT1
  source 1..20: mol_type = other DNA
agtggggtcctcactgcact

SEQ ID NO: 146 - DNA, 20 bp, synthetic construct
  misc_feature 1..20: note = Synthetic- MIP_OT2
  source 1..20: mol_type = other DNA
tgtggggcactcactgcgct

SEQ ID NO: 147 - DNA, 20 bp, synthetic construct
  misc_feature 1..20: note = Synthetic- FAM163_OT1
  source 1..20: mol_type = other DNA
ctgcagggcccgctggagag

SEQ ID NO: 148 - DNA, 20 bp, synthetic construct
  misc_feature 1..20: note = Synthetic- FAM163_OT2
  source 1..20: mol_type = other DNA
ctgcaggggacactggtgag

SEQ ID NO: 149 - DNA, 20 bp, synthetic construct
  misc_feature 1..20: note = Synthetic- OMP_OT1
  source 1..20: mol_type = other DNA
aggctgtagcccctgctgct

SEQ ID NO: 150 - DNA, 20 bp, synthetic construct
  misc_feature 1..20: note = Synthetic- OMP_OT2
  source 1..20: mol_type = other DNA
gaactacagccactgctgct

SEQ ID NO: 151 - DNA, 20 bp, synthetic construct
  misc_feature 1..20: note = Synthetic- FANCF_OT1
  source 1..20: mol_type = other DNA
gctgcagaagggattccaag

SEQ ID NO: 152 - DNA, 20 bp, synthetic construct
  misc_feature 1..20: note = synthetic- MAPRE_OT1
  source 1..20: mol_type = other DNA
atctctgcagataatccctg

SEQ ID NO: 153 - DNA, 20 bp, synthetic construct
  misc_feature 1..20: note = Synthetic- OPN1SW_OT1
  source 1..20: mol_type = other DNA
ttagaggcaatgtggtactg

SEQ ID NO: 154 - DNA, 20 bp, synthetic construct
  misc_feature 1..20: note = Synthetic- VEGFA_OT1
  source 1..20: mol_type = other DNA
tgtgggtgagtgtgtgcgtg

SEQ ID NO: 155 - DNA, 20 bp, synthetic construct
  misc_feature 1..20: note = Synthetic- MIP_OT1 F
  source 1..20: mol_type = other DNA
ctcacagcaaggtcgaccac

SEQ ID NO: 156 - DNA, 20 bp, synthetic construct
  misc_feature 1..20: note = Synthetic- MIP_OT1 R
  source 1..20: mol_type = other DNA
cacccctacacactgccttt

SEQ ID NO: 157 - DNA, 30 bp, synthetic construct
  misc_feature 1..30: note = Synthetic- MIP_OT1 primer seq
  source 1..30: mol_type = other DNA
cattcgaaatcctatgctgagctttcatag

SEQ ID NO: 158 - DNA, 20 bp, synthetic construct
  misc_feature 1..20: note = Synthetic- MIP_OT2 F
  source 1..20: mol_type = other DNA
cggctccagtgctctttctt

SEQ ID NO: 159 - DNA, 20 bp, synthetic construct
  misc_feature 1..20: note = Synthetic- MIP_OT2 R
  source 1..20: mol_type = other DNA
ggagggtacgcaaggtttgg

SEQ ID NO: 160 - DNA, 22 bp, synthetic construct
  misc_feature 1..22: note = Synthetic- MIP_OT2 primer seq
  source 1..22: mol_type = other DNA
gcctttctgactcccatccttc

SEQ ID NO: 161 - DNA, 20 bp, synthetic construct
  misc_feature 1..20: note = Synthetic- FAM163_OT1 F
  source 1..20: mol_type = other DNA
gtggataggagcatctgccc

SEQ ID NO: 162 - DNA, 20 bp, synthetic construct
  misc_feature 1..20: note = Synthetic- FAM163_OT1 R
  source 1..20: mol_type = other DNA
gtgggagaaggaggtcatgc

SEQ ID NO: 163 - DNA, 23 bp, synthetic construct
  misc_feature 1..23: note = Synthetic- FAM163_OT1 primer seq
  source 1..23: mol_type = other DNA
cctccccatatgcttggagtaag

SEQ ID NO: 164 - DNA, 20 bp, synthetic construct
  misc_feature 1..20: note = Synthetic- FAM163_OT2 F
  source 1..20: mol_type = other DNA
gcccacatttgcactgactc

SEQ ID NO: 165 - DNA, 20 bp, synthetic construct
  misc_feature 1..20: note = Synthetic- FAM163_OT2 R
  source 1..20: mol_type = other DNA
gatcatggtgatgtgcgcac

SEQ ID NO: 166 - DNA, 30 bp, synthetic construct
  misc_feature 1..30: note = Synthetic- FAM163_OT2 primer seq
  source 1..30: mol_type = other DNA
agacaagacaccacagcaattccaattttg

SEQ ID NO: 167 - DNA, 20 bp, synthetic construct
  misc_feature 1..20: note = Synthetic- OMP_OT1 F
  source 1..20: mol_type = other DNA
agatcctgggggtctctgtg

SEQ ID NO: 168 - DNA, 20 bp, synthetic construct
  misc_feature 1..20: note = Synthetic- OMP_OT1 R
  source 1..20: mol_type = other DNA
cgcctgcttatcatttgggc

SEQ ID NO: 169 - DNA, 30 bp, synthetic construct
  misc_feature 1..30: note = Synthetic- OMP_OT1 primer seq
  source 1..30: mol_type = other DNA
gaactagagacttatgagtggttctaagat

SEQ ID NO: 170 - DNA, 20 bp, synthetic construct
  misc_feature 1..20: note = Synthetic- OMP_OT2 F
  source 1..20: mol_type = other DNA
ttgcaacaccagggctttct

SEQ ID NO: 171 - DNA, 20 bp, synthetic construct
  misc_feature 1..20: note = Synthetic- OMP_OT2 R
  source 1..20: mol_type = other DNA
cttcacaggcttcagggagg

SEQ ID NO: 172 - DNA, 30 bp, synthetic construct
  misc_feature 1..30: note = Synthetic- OMP_OT2 primer seq
  source 1..30: mol_type = other DNA
tagcatttccttctttagaggttgattatg

SEQ ID NO: 173 - DNA, 24 bp, synthetic construct
  misc_feature 1..24: note = Synthetic- FANCF_OT1 F
  source 1..24: mol_type = other DNA
agtttcacatccctgtcttacctc

SEQ ID NO: 174 - DNA, 24 bp, synthetic construct
  misc_feature 1..24: note = Synthetic- FANCF_OT1 R
  source 1..24: mol_type = other DNA
agactcacaacatccatcagaaca

SEQ ID NO: 175 - DNA, 24 bp, synthetic construct
  misc_feature 1..24: note = Synthetic- FANCF_OT1 primer seq
  source 1..24: mol_type = other DNA
agtttcacatccctgtcttacctc

SEQ ID NO: 176 - DNA, 21 bp, synthetic construct
  misc_feature 1..21: note = Synthetic- MAPRE_OT1 F
  source 1..21: mol_type = other DNA
acagtttgtgggctttttggt

SEQ ID NO: 177 - DNA, 20 bp, synthetic construct
  misc_feature 1..20: note = Synthetic- MAPRE_OT1 R
  source 1..20: mol_type = other DNA
gcattctgccctgtttgtgg

SEQ ID NO: 178 - DNA, 24 bp, synthetic construct
  misc_feature 1..24: note = Synthetic- MAPRE_OT1 primer seq
  source 1..24: mol_type = other DNA
cattttgagcaaggtcagaaggac

SEQ ID NO: 179 - DNA, 20 bp, synthetic construct
  misc_feature 1..20: note = Synthetic- OPN1SW_OT1 F
  source 1..20: mol_type = other DNA
tggccataggaagcacagtc

SEQ ID NO: 180 - DNA, 20 bp, synthetic construct
  misc_feature 1..20: note = Synthetic- OPN1SW_OT1 R
  source 1..20: mol_type = other DNA
atgatccccctgtctctgct

SEQ ID NO: 181 - DNA, 24 bp, synthetic construct
  misc_feature 1..24: note = Synthetic- OPN1SW_OT1 primer seq
  source 1..24: mol_type = other DNA
ctacctccctctccttagcttctc

SEQ ID NO: 182 - DNA, 24 bp, synthetic construct
  misc_feature 1..24: note = Synthetic- VEGFA_OT1 F
  source 1..24: mol_type = other DNA
agggacttgagtatctgcagtttt

SEQ ID NO: 183 - DNA, 24 bp, synthetic construct
  misc_feature 1..24: note = Synthetic- VEGFA_OT1 R
  source 1..24: mol_type = other DNA
tgaagagatatctgcaccctcatg

SEQ ID NO: 184 - DNA, 24 bp, synthetic construct
  misc_feature 1..24: note = Synthetic- VEGFA_OT1 primer seq
  source 1..24: mol_type = other DNA
agggacttgagtatctgcagtttt

SEQ ID NO: 185 - DNA, 23 bp, synthetic construct
  misc_feature 1..23: note = Synthetic- sgRNA_F
  source 1..23: mol_type = other DNA
agagctagaaatagcaagttaaa

SEQ ID NO: 186 - DNA, 16 bp, synthetic construct
  misc_feature 1..16: note = Synthetic- sgRNA_R
  source 1..16: mol_type = other DNA
gactcggtgccacttt

SEQ ID NO: 187 - RNA, 20 nt, synthetic construct
  misc_feature 1..20: note = Synthetic- CRISPR ON V4 sgRNA variants
  source 1..20: mol_type = other RNA
tctccctgagcttcagggag

SEQ ID NO: 188 - RNA, 100 nt, synthetic construct; identical to SEQ ID NO: 8
  source 1..100: mol_type = other RNA

SEQ ID NO: 189 - RNA, 100 nt, synthetic construct
  source 1..100: mol_type = other RNA
  modified_base 49: mod_base = OTHER; note = Y = dT - C6NH2
nnnnnnnnnnnnnnnnnnnngatttagagctagaaatagcaagttaaaytaaggctagtccgttatcaacttgaaaaagtggcaccgagtcggtgctttt

SEQ ID NO: 190 - DNA, 20 bp, synthetic construct
  source 1..20: mol_type = other DNA
gatctccacataggggccag

SEQ ID NO: 191 - RNA, 100 nt, synthetic construct
  source 1..100: mol_type = other RNA
  modified_base 49: mod_base = OTHER; note = Y = dT - C6NH2
  misc_feature 49: note = maleimide
gctgcagaagggattccatggatttagagctagaaatagcaagttaaaytaaggctagtccgttatcaacttgaaaaagtggcaccgagtcggtgctttt

SEQ ID NO: 192 - RNA, 100 nt, synthetic construct
  source 1..100: mol_type = other RNA
  modified_base 49: mod_base = OTHER; note = Y = dT - C6NH2
  modified_base 49: mod_base = OTHER; note = maleimide
gatctccacataggggccaggatttagagctagaaatagcaagttaaaytaaggctagtccgttatcaacttgaaaaagtggcaccgagtcggtgctttt

SEQ ID NO: 193 - DNA, 20 bp, synthetic construct
  source 1..20: mol_type = other DNA
gcccagacatccaaacctga

SEQ ID NO: 194 - DNA, 20 bp, synthetic construct
  source 1..20: mol_type = other DNA
cgtggagagcagattcctcc

SEQ ID NO: 195 - DNA, 22 bp, synthetic construct
  source 1..22: mol_type = other DNA
cccagacatccaaacctgactc